(19) United States
(12) Patent Application Publication
(10) Pub. No.: US 2002/0098546 A1
(43) Pub. Date: Jul. 25, 2002

(54) Canine TAg1 proteins, nucleic acid molecules, and uses thereof

(76) Inventors: Eric R. Weber, Fort Collins, CO (US); Wayne Arthur Jensen, Wellington, CO (US); Ramaswamy Chandrashekar, Fort Collins, CO (US)

Correspondence Address: Heska Corporation, Intellectual Property Dept., 1613 Prospect Parkway, Fort Collins, CO, 80525, US

(21) Appl. No.: 09/918,063

(22) Filed: Jul. 30, 2001

Related U.S. Application Data
(60) Provisional application No. 60/224,655, filed on Aug. 11, 2000.

(51) Int. Cl. C12P 21/02; C12Q 1/68; G01N 33/53; C12N 9/00; C12N 7/00; C12N 5/06; C12N 15/86

(52) U.S. Cl. 435/69.1; 435/183; 435/6; 435/7.1; 435/350; 536/23.2; 435/235.1; 435/456; 530/388.1

(57) ABSTRACT

The present invention relates to canine TAg1 proteins; to canine TAg1 nucleic acid molecules, including those that encode such TAg1 proteins; to antibodies raised against such TAg1 proteins; and to compounds that inhibit TAg1 activity. The present invention also includes methods to obtain such proteins, nucleic acid molecules, antibodies, and inhibitory compounds. Also included in the present invention are compositions and methods for detecting the presence of TAg1 protein or anti-TAg1 antibodies in an animal. The present invention also includes therapeutic compositions comprising such proteins, nucleic acid molecules, antibodies and/or inhibitory compounds as well as the use of such therapeutic compositions to protect animals from disease.

### CROSS REFERENCE TO RELATED APPLICATION

This application claims priority to U.S. Provisional patent application Ser. No. 60/224,655, filed Aug. 11, 2000, entitled “CANINE TAg1 PROTEINS, NUCLEIC ACID MOLECULES, AND USES THEREOF.”

### FIELD OF THE INVENTION

The present invention relates to canine tumor antigen 1 (TAg1) nucleic acid molecules, proteins encoded by such nucleic acid molecules and antibodies raised against such proteins. The present invention also includes therapeutic compositions comprising such nucleic acid molecules, proteins, and/or antibodies, as well as their use to protect animals, including human patients, from disease. The present invention also includes the use of such nucleic acid molecules, proteins, and/or antibodies, in the detection of disease.

### BACKGROUND OF THE INVENTION

Cancer is among the leading causes of death in companion animals, having a frequency and complexity similar to that seen in humans. As in humans, early detection and treatment can greatly enhance the survival rate of companion animals. Currently, cancer is detected primarily by imaging techniques such as x-ray, CAT scan and NMR. While such techniques are rapidly becoming more routine and sensitive, they remain moderately expensive to perform and fail to detect some cancers. Current cancer treatments typically involve invasive and/or toxic treatments such as surgery and chemotherapy.

Previous researchers have speculated that oncofetal protein (OFP), a secreted cancer cell product, may be a useful agent for the detection of cancer as well as a useful target in the treatment of cancer, see for example U.S. Pat. Nos. 4,746,539 and 5,650,300 to Webb et al. and U.S. Pat. Nos. 5,310,653, 5,411,868, and 5,773,215 to Hanausek-Walaszek et al. However, to date no single treatment cures cancer and no single diagnostic test is available for early detection for all types of cancer.

As such, there remains a continuing need to identify molecules which may be useful as cancer-specific markers for early detection of disease. Such molecules would preferably also be useful for disease prevention and treatment.

### SUMMARY OF THE INVENTION

The present invention relates to a novel product and a process to detect cancer in animals. According to the present invention there are provided a canine TAg1 protein, a canine TAg1 nucleic acid molecule, including those that encode such a protein; and an antibody raised against such a canine TAg1 protein (i.e., an anti-canine TAg1 antibody).

The present invention also includes methods to obtain and/or identify such a protein, nucleic acid molecule, and antibody. Also included in the present invention is a therapeutic composition comprising such a protein, nucleic acid molecule, and/or antibody, as well as use of such a therapeutic composition to protect animals from disease.

A preferred canine TAg1 nucleic acid molecule of the present invention includes an isolated nucleic acid molecule selected from the group consisting of: (a) a nucleic acid molecule that hybridizes with a nucleic acid molecule having a nucleic acid sequence selected from the group consisting of SEQ ID NO:8, SEQ ID NO:10, SEQ ID NO:23, SEQ ID NO:25, SEQ ID NO:32, SEQ ID NO:34, SEQ ID NO:35, and SEQ ID NO:37, under conditions comprising (i) hybridizing in a solution comprising 1×SSC in the absence of nucleic acid helix destabilizing agents, at a temperature of about 37° C. and (ii) washing in a solution comprising 1×SSC and in the absence of nucleic acid helix destabilizing agents, at a temperature of about 56° C.; (b) a nucleic acid molecule comprising a nucleic acid sequence selected from the group consisting of SEQ ID NO:11, SEQ ID NO:13, SEQ ID NO:26, SEQ ID NO:28, SEQ ID NO:38, SEQ ID NO:40, SEQ ID NO:41, and SEQ ID NO:43, and fragments thereof, wherein said fragment is at least about 20 nucleotides in length; and (c) a nucleic acid molecule comprising a nucleic acid sequence selected from the group consisting of SEQ ID NO:2, SEQ ID NO:4, SEQ ID NO:5, SEQ ID NO:7, SEQ ID NO:14, SEQ ID NO:16, SEQ ID NO:17, SEQ ID NO:19, SEQ ID NO:20, SEQ ID NO:22, SEQ ID NO:29, SEQ ID NO:31, SEQ ID NO:44, SEQ ID NO:46, SEQ ID NO:47, SEQ ID NO:49, SEQ ID NO:50, SEQ ID NO:52, SEQ ID NO:53, SEQ ID NO:55, SEQ ID NO:56, SEQ ID NO:58, SEQ ID NO:59, SEQ ID NO:61, SEQ ID NO:62, SEQ ID NO:64, SEQ ID NO:65, SEQ ID NO:67, SEQ ID NO:68, SEQ ID NO:70, SEQ ID NO:71, SEQ ID NO:72, SEQ ID NO:73, SEQ ID NO:75, SEQ ID NO:76, and SEQ ID NO:77, and fragments thereof, wherein said fragment is at least about 40 nucleotides in length.

A particularly preferred canine TAg1 nucleic acid molecule comprises nucleic acid sequence SEQ ID NO:2, SEQ ID NO:4, SEQ ID NO:5, SEQ ID NO:7, SEQ ID NO:8, SEQ ID NO:10, SEQ ID NO:11, SEQ ID NO:13, SEQ ID NO:14, SEQ ID NO:16, SEQ ID NO:17, SEQ ID NO:19, SEQ ID NO:20, SEQ ID NO:22, SEQ ID NO:23, SEQ ID NO:25, SEQ ID NO:26, SEQ ID NO:28, SEQ ID NO:29, SEQ ID NO:31, SEQ ID NO:32, SEQ ID NO:34, SEQ ID NO:35, SEQ ID NO:37, SEQ ID NO:38, SEQ ID NO:40, SEQ ID NO:41, SEQ ID NO:43, SEQ ID NO:44, SEQ ID NO:46, SEQ ID NO:47, SEQ ID NO:49, SEQ ID NO:50, SEQ ID NO:52, SEQ ID NO:53, SEQ ID NO:55, SEQ ID NO:56, SEQ ID NO:58, SEQ ID NO:59, SEQ ID NO:61, SEQ ID NO:62, SEQ ID NO:64, SEQ ID NO:65, SEQ ID NO:67, SEQ ID NO:68, SEQ ID NO:70, SEQ ID NO:71, SEQ ID NO:72, SEQ ID NO:73, SEQ ID NO:75, SEQ ID NO:76, and/or SEQ ID NO:77.

A particularly preferred canine TAg1 nucleic acid molecule comprises an isolated nucleic acid molecule selected from the group consisting of: (a) an isolated nucleic acid molecule encoding a protein comprising an amino acid sequence selected from the group consisting of SEQ ID NO:3, SEQ ID NO:9, SEQ ID NO:12, SEQ ID NO:15, SEQ ID NO:18, SEQ ID NO:24, SEQ ID NO:27, SEQ ID NO:30, SEQ ID NO:33, SEQ ID NO:36, SEQ ID NO:39, SEQ ID NO:42, SEQ ID NO:45, SEQ ID NO:48, SEQ ID NO:51, SEQ ID NO:54, SEQ ID NO:57, SEQ ID NO:60, SEQ ID NO:63, SEQ ID NO:66, SEQ ID NO:69, and SEQ ID NO:74; (b) an isolated nucleic acid molecule encoding a protein comprising an at least 6 consecutive amino acid portion identical in sequence to an at least 6 consecutive amino acid portion of a sequence selected from the group consisting of SEQ ID NO:9, SEQ ID NO:24, SEQ ID NO:33, and SEQ ID NO:36; (c) an isolated nucleic acid molecule encoding a protein comprising an at least 15 consecutive amino acid portion identical in sequence to an at least 15 consecutive amino acid portion of a sequence selected from the group consisting of SEQ ID NO:12, SEQ ID NO:27, SEQ ID NO:39, and SEQ ID NO:42; (d) an isolated nucleic acid molecule encoding a protein comprising an at least 42 consecutive amino acid portion identical in sequence to an at least 42 consecutive amino acid portion of a sequence selected from the group consisting of SEQ ID NO:3, SEQ ID NO:15, SEQ ID NO:18, SEQ ID NO:30, SEQ ID NO:45, SEQ ID NO:48, SEQ ID NO:51, SEQ ID NO:54, SEQ ID NO:57, SEQ ID NO:60, SEQ ID NO:63, SEQ ID NO:66, SEQ ID NO:69, and SEQ ID NO:74; and (e) a nucleic acid molecule complementary to a nucleic acid molecule of (a), (b), (c) or (d).

The present invention also relates to a recombinant molecule, a recombinant virus and a recombinant cell that includes an isolated TAg1 nucleic acid molecule of the present invention. Also included are methods to produce such a nucleic acid molecule, recombinant molecule, recombinant virus and recombinant cell.

Another embodiment of the present invention includes a canine TAg1 protein. A preferred canine TAg1 protein includes an isolated protein selected from the group consisting of: (a) a protein comprising an amino acid sequence selected from the group consisting of SEQ ID NO:3, SEQ ID NO:9, SEQ ID NO:12, SEQ ID NO:15, SEQ ID NO:18, SEQ ID NO:24, SEQ ID NO:27, SEQ ID NO:30, SEQ ID NO:33, SEQ ID NO:36, SEQ ID NO:39, SEQ ID NO:42, SEQ ID NO:45, SEQ ID NO:48, SEQ ID NO:51, SEQ ID NO:54, SEQ ID NO:57, SEQ ID NO:60, SEQ ID NO:63, SEQ ID NO:66, SEQ ID NO:69, and SEQ ID NO:74; (b) a protein comprising an at least 6 consecutive amino acid portion identical in sequence to an at least 6 consecutive amino acid portion of a sequence selected from the group consisting of SEQ ID NO:9, SEQ ID NO:24, SEQ ID NO:33, and SEQ ID NO:36; (c) a protein comprising an at least 15 consecutive amino acid portion identical in sequence to an at least 15 consecutive amino acid portion of a sequence selected from the group consisting of SEQ ID NO:12, SEQ ID NO:27, SEQ ID NO:39, and SEQ ID NO:42 and (d) a protein comprising an at least 42 consecutive amino acid portion identical in sequence to an at least 42 consecutive amino acid portion of a sequence selected from the group consisting of SEQ ID NO:3, SEQ ID NO:15, SEQ ID NO:18, SEQ ID NO:30, SEQ ID NO:45, SEQ ID NO:48, SEQ ID NO:51, SEQ ID NO:54, SEQ ID NO:57, SEQ ID NO:60, SEQ ID NO:63, SEQ ID NO:66, SEQ ID NO:69, and SEQ ID NO:74.

A particularly preferred canine TAg1 protein comprises an amino acid sequence SEQ ID NO:3, SEQ ID NO:9, SEQ ID NO:12, SEQ ID NO:15, SEQ ID NO:18, SEQ ID NO:24, SEQ ID NO:27, SEQ ID NO:30, SEQ ID NO:33, SEQ ID NO:36, SEQ ID NO:39, SEQ ID NO:42, SEQ ID NO:45, SEQ ID NO:48, SEQ ID NO:51, SEQ ID NO:54, SEQ ID NO:57, SEQ ID NO:60, SEQ ID NO:63, SEQ ID NO:66, SEQ ID NO:69, and/or SEQ ID NO:74. The present invention further relates to isolated antibodies that selectively bind to canine TAg1 proteins of the present invention, or to a mimetope thereof.

Yet another embodiment of the present invention is a therapeutic composition that is capable of protecting an animal from disease. Such a therapeutic composition includes one or more of the following protective compounds: a canine TAg1 protein of the present invention or a mimetope thereof; an isolated canine TAg1 nucleic acid molecule of the present invention; an isolated antibody that selectively binds to a canine TAg1 protein of the present invention; and/or a compound capable of inhibiting TAg1 function. A preferred therapeutic composition of the present invention also includes an excipient, an adjuvant and/or a carrier. Preferred TAg1 nucleic acid molecule therapeutic compositions of the present invention include genetic vaccines, recombinant virus vaccines and recombinant cell vaccines. Also included in the present invention is a method to protect an animal from disease, comprising the step of administering to the animal a therapeutic composition of the present invention.

It is also within the scope of the present invention to use isolated proteins, mimetopes, nucleic acid molecules and antibodies of the present invention as diagnostic reagents to detect disease. One embodiment of the present invention is a method to detect disease which includes the steps of: (a) contacting an isolated anti-TAg1 antibody with a putative TAg1 protein-containing composition under conditions suitable for formation of a anti-TAg1:TAg1 protein complex; and (b) detecting the presence of TAg1 protein by detecting the anti-TAg1: TAg1 protein complex. Presence of such a anti-TAg1:TAg1 protein complex indicates that the animal is producing TAg1 protein. Another embodiment of the present invention is a method to detect disease which includes the steps of: (a) contacting an isolated TAg1 protein with a putative anti-TAg1 antibody-containing composition under conditions suitable for formation of a anti-TAg1 :TAg1 protein complex; and (b) detecting the presence of anti-TAg1 antibody by detecting the anti-TAg1:TAg1 protein complex. Presence of such an anti-TAg1:TAg1 protein complex indicates that the animal is producing anti-TAg1 antibody in response to the presence of TAg1 protein.

The present invention also includes kits to detect TAg1 protein based upon detection methods disclosed herein. Suitable and preferred TAg1 molecules to detect using such a kit are disclosed herein. A preferred kit of the present invention includes those in which the anti-TAg1 is immobilized on a support substrate using a method as described herein. In another embodiment, a preferred kit comprises a lateral flow apparatus, a flow through apparatus or a microtiter dish. A preferred format for a kit is an ELISA or a biotin, avidin or metal-linked immunosorbant assay. A method and kit of the present invention are useful for diagnosing abnormal conditions in animals that are associated with TAg1 protein.

Another embodiment of the present invention is a method and a kit to identify a compound capable of inhibiting TAg1 protein activity. Such a method includes the steps of (a) contacting (e.g., combining, mixing) an isolated TAg1 protein of the present invention, with a putative inhibitory compound under conditions in which, in the absence of the compound, the protein has TAg1 protein activity, and (b) determining if the putative inhibitory compound inhibits the activity. Such a kit comprises an isolated TAg1 protein of the present invention, and a means for determining inhibition of TAg1 protein activity, where the means enables detection of inhibition.

### DETAILED DESCRIPTION OF THE INVENTION

The present invention provides for isolated canine Tumor Antigen-1 (TAg1) proteins, isolated canine TAg1 nucleic acid molecules, isolated antibodies directed against canine TAg1 proteins, and compounds able to inhibit canine TAg1 protein function (i.e., inhibitory compounds). As used herein, the terms isolated canine TAg1 proteins and isolated canine TAg1 nucleic acid molecules refer to TAg1 proteins and TAg1 nucleic acid molecules derived from a canine; as such the proteins and nucleic acid molecules can be isolated from a canine or prepared recombinantly or synthetically. Canine TAg1 nucleic acid molecules of known length are denoted “ncaTAg1_{#}” (for example ncaTAg1₁₂₃₆) wherein “#” refers to the number of nucleotides in that molecule, and canine TAg1 proteins of known length are denoted “PcaTAg1_{#}” (for example PcaTAg1₃₂₆) wherein “#” refers to the number of amino acid residues in that molecule. As described in the examples below, canine TAg1 nucleic acid molecule and protein names may also contain letters denoting the tissue from which the molecule was initially isolated, for example, ncapTAg1₁₂₃₆ was initially isolated from a canine placental cDNA library and ncasTAg1₁₂₆₉ was initially isolated from a canine soft tissue sarcoma cDNA library. The proteins and nucleic acid molecules of the present invention can be obtained from their natural source, or can be produced using, for example, recombinant nucleic acid technology or chemical synthesis. Also included in the present invention is the use of these proteins, nucleic acid molecules, antibodies, and inhibitory compounds as therapeutic compositions to protect animals from disease, as diagnostic compositions to detect disease, as well as in other applications, such as those disclosed below.

One embodiment of the present invention is an isolated protein that includes a canine TAg1 protein. It is to be noted that the term “a” or “an” entity refers to one or more of that entity; for example, a protein, a nucleic acid molecule, an antibody, an inhibitor, a compound or a therapeutic composition refers to “one or more” or “at least one” protein, nucleic acid molecule, antibody, inhibitor, compound or therapeutic composition respectively. As such, the terms “a” (or “an”), “one or more” and “at least one” can be used interchangeably herein. It is also to be noted that the terms “comprising”, “including”, and “having” can be used interchangeably. According to the present invention, an isolated, or biologically pure, protein, is a protein that has been removed from its natural milieu. As such, “isolated” and “biologically pure” do not necessarily reflect the extent to which the protein has been purified. An isolated protein of the present invention can be obtained from its natural source, can be produced using recombinant DNA technology, or can be produced by chemical synthesis.

As used herein, an isolated canine TAg1 protein of the present invention can be a full-length protein or any homologue of such a protein. An isolated protein of the present invention, including a homologue, can be identified in a straight-forward manner by the protein's ability to elicit an immune response against a canine TAg1 protein, and preferably against a protein having SEQ ID NO:3, SEQ ID NO:9, SEQ ID NO:12, SEQ ID NO:15, SEQ ID NO:18, SEQ ID NO:24, SEQ ID NO:27, SEQ ID NO:30, SEQ ID NO:33, SEQ ID NO:36, SEQ ID NO:39, SEQ ID NO:42, SEQ ID NO:45, SEQ ID NO:48, SEQ ID NO:51, SEQ ID NO:54, SEQ ID NO:57, SEQ ID NO:60, SEQ ID NO:63, SEQ ID NO:66, SEQ ID NO:69, and/or SEQ ID NO:74, or by the protein's TAg1 activity. Examples of canine TAg1 homologue proteins include canine TAg1 proteins in which amino acids have been deleted (e.g., a truncated version of the protein, such as a peptide), inserted, inverted, substituted and/or derivatized (e.g., by glycosylation, phosphorylation, acetylation, myristoylation, prenylation, palmitoylation, amidation and/or addition of glycerophosphatidyl inositol) such that the homologue includes at least one epitope capable of eliciting an immune response against a canine TAg1 protein, and/or of binding to an antibody directed against a canine TAg1 protein. For example, when a homologue is administered to an animal as an immunogen, using techniques known to those skilled in the art, the animal will produce an immune response against at least one epitope of a natural canine TAg1 protein. The ability of a protein to effect an immune response can be measured using techniques known to those skilled in the art. Methods to determine binding between proteins and antibodies are also know to those skilled in the art. As used herein, the term “epitope” refers to the smallest portion of a protein or other antigen capable of selectively binding to the antigen binding site of an antibody or a T-cell receptor. It is well accepted by those skilled in the art that the minimal size of a protein epitope is about four to six amino acids. As is appreciated by those skilled in the art, an epitope can include amino acids that naturally are contiguous to each other as well as amino acids that, due to the tertiary structure of the natural protein, are in sufficiently close proximity to form an epitope. According to the present invention, an epitope includes a portion of a protein comprising at least about 4 amino acids, at least about 5 amino acids, at least about 6 amino acids, at least about 10 amino acids, at least about 15 amino acids, at least about 20 amino acids, at least about 25 amino acids, at least about 30 amino acids, at least about 35 amino acids, at least about 40 amino acids or at least about 50 amino acids. In one embodiment of the present invention a canine homologue protein has TAg1 proteinactivity.

Canine TAg1 homologue proteins can be the result of natural allelic variation or natural mutation. Canine TAg1 protein homologues of the present invention can also be produced using techniques known in the art including, but not limited to, direct modifications to the protein or modifications to the gene or cDNA encoding the protein using, for example, classic or recombinant DNA techniques to effect random or targeted mutagenesis.

Canine TAg1 proteins of the present invention are encoded by canine TAg1 nucleic acid molecules. As used herein, a canine TAg1 nucleic acid molecule includes nucleic acid sequences related to a natural canine TAg1 gene. Examples of canids from which to isolate proteins, genes and nucleic acid molecules are disclosed herein. As used herein, a canine TAg1 gene includes all regions such as regulatory regions that control production of the canine TAg1 protein encoded by the gene (such as, but not limited to, transcription, translation or post-translation control regions) as well as the coding region itself, and any introns or non-translated coding regions. As used herein, a gene that “includes” or “comprises” a sequence may include that sequence in one contiguous array, or may include the sequence as fragmented exons. As used herein, the term “coding region” refers to a continuous linear array of nucleotides that translates into a protein. A full-length coding region is that coding region that is translated into a full-length, i.e., a complete protein as would be initially translated in its natural millieu, prior to any post-translational modifications.

One embodiment of the present invention is a canine TAg1 gene that includes the nucleic acid sequence SEQ ID NO:2, SEQ ID NO:4, SEQ ID NO:5, SEQ ID NO:7, SEQ ID NO:8, SEQ ID NO:10, SEQ ID NO:11, SEQ ID NO:13, SEQ ID NO:14, SEQ ID NO:16, SEQ ID NO:17, SEQ ID NO:19, SEQ ID NO:20, SEQ ID NO:22, SEQ ID NO:23, SEQ ID NO:25, SEQ ID NO:26, SEQ ID NO:28, SEQ ID NO:29, SEQ ID NO:31, SEQ ID NO:32, SEQ ID NO:34, SEQ ID NO:35, SEQ ID NO:37, SEQ ID NO:38, SEQ ID NO:40, SEQ ID NO:41, SEQ ID NO:43, SEQ ID NO:44, SEQ ID NO:46, SEQ ID NO:47, SEQ ID NO:49, SEQ ID NO:50, SEQ ID NO:52, SEQ ID NO:53, SEQ ID NO:55, SEQ ID NO:56, SEQ ID NO:58, SEQ ID NO:59, SEQ ID NO:61, SEQ ID NO:62, SEQ ID NO:64, SEQ ID NO:65, SEQ ID NO:67, SEQ ID NO:68, SEQ ID NO:70, SEQ ID NO:71, SEQ ID NO:72, SEQ ID NO:73, SEQ ID NO:75, SEQ ID NO:76, and/or SEQ ID NO:77, as well as the complements of any of these nucleic acid sequences. These nucleic acid sequences are further described herein. For example, nucleic acid sequence SEQ ID NO:2 represents the deduced sequence of the coding strand of a cDNA (complementary DNA) denoted herein as canine TAg1 nucleic acid molecule ncapTAg1₁₂₃₆, the production of which is disclosed in the Examples. Nucleic acid molecule ncapTAg1₁₂₃₆ comprises an apparent full-length coding region. The complement of SEQ ID NO:2 (represented herein by SEQ ID NO:4) refers to the nucleic acid sequence of the strand that is fully (i.e. 100%) complementary to the strand having SEQ ID NO:2, which can easily be determined by those skilled in the art. Likewise, a nucleic acid sequence complement of any nucleic acid sequence of the present invention refers to the nucleic acid sequence of the nucleic acid strand that is fully complementary to (i.e., can form a complete, or full, double helix with) the strand for which the sequence is cited. It should be noted that since nucleic acid sequencing technology is not entirely error-free, SEQ ID NO:2 (as well as other nucleic acid and protein sequences presented herein) represents an apparent nucleic acid sequence of the nucleic acid molecule encoding a caTAg1 protein of the present invention.

In another embodiment, a caTAg1 gene or nucleic acid molecule can be an allelic variant that includes a similar but not identical sequence to SEQ ID NO:2, SEQ ID NO:4, SEQ ID NO:5, SEQ ID NO:7, SEQ ID NO:8, SEQ ID NO:10, SEQ ID NO: 11, SEQ ID NO:13, SEQ ID NO:14, SEQ ID NO:16, SEQ ID NO:17, SEQ ID NO:19, SEQ ID NO:20, SEQ ID NO:22, SEQ ID NO:23, SEQ ID NO:25, SEQ ID NO:26, SEQ ID NO:28, SEQ ID NO:29, SEQ ID NO:31, SEQ ID NO:32, SEQ ID NO:34, SEQ ID NO:35, SEQ ID NO:37, SEQ ID NO:38, SEQ ID NO:40, SEQ ID NO:41, SEQ ID NO:43, SEQ ID NO:44, SEQ ID NO:46, SEQ ID NO:47, SEQ ID NO:49, SEQ ID NO:50, SEQ ID NO:52, SEQ ID NO:53, SEQ ID NO:55, SEQ ID NO:56, SEQ ID NO:58, SEQ ID NO:59, SEQ ID NO:61, SEQ ID NO:62, SEQ ID NO:64, SEQ ID NO:65, SEQ ID NO:67, SEQ ID NO:68, SEQ ID NO:70, SEQ ID NO:71, SEQ ID NO:72, SEQ ID NO:73, SEQ ID NO:75, SEQ ID NO:76, and/or SEQ ID NO:77, or any other nucleic acid sequence cited herein. For example, an allelic variant of a caTAg1 gene including SEQ ID NO:2, SEQ ID NO:4, SEQ ID NO:5, SEQ ID NO:7, SEQ ID NO:8, SEQ ID NO:10, SEQ ID NO:11, SEQ ID NO:13, SEQ ID NO:14, SEQ ID NO:16, SEQ ID NO:17, SEQ ID NO:19, SEQ ID NO:20, SEQ ID NO:22, SEQ ID NO:23, SEQ ID NO:25, SEQ ID NO:26, SEQ ID NO:28, SEQ ID NO:29, SEQ ID NO:31, SEQ ID NO:32, SEQ ID NO:34, SEQ ID NO:35, SEQ ID NO:37, SEQ ID NO:38, SEQ ID NO:40, SEQ ID NO:41, SEQ ID NO:43, SEQ ID NO:44, SEQ ID NO:46, SEQ ID NO:47, SEQ ID NO:49, SEQ ID NO:50, SEQ ID NO:52, SEQ ID NO:53, SEQ ID NO:55, SEQ ID NO:56, SEQ ID NO:58, SEQ ID NO:59, SEQ ID NO:61, SEQ ID NO:62, SEQ ID NO:64, SEQ ID NO:65, SEQ ID NO:67, SEQ ID NO:68, SEQ ID NO:70, SEQ ID NO:71, SEQ ID NO:72, SEQ ID NO:73, SEQ ID NO:75, SEQ ID NO:76, and/or SEQ ID NO:77, is a gene that occurs at essentially the same locus (or loci) in the genome as the gene including SEQ ID NO:2, SEQ ID NO:4, SEQ ID NO:5, SEQ ID NO:7, SEQ ID NO:8, SEQ ID NO:10, SEQ ID NO:11, SEQ ID NO:13, SEQ ID NO:14, SEQ ID NO:16, SEQ ID NO:17, SEQ ID NO:19, SEQ ID NO:20, SEQ ID NO:22, SEQ ID NO:23, SEQ ID NO:25, SEQ ID NO:26, SEQ ID NO:28, SEQ ID NO:29, SEQ ID NO:31, SEQ ID NO:32, SEQ ID NO:34, SEQ ID NO:35, SEQ ID NO:37, SEQ ID NO:38, SEQ ID NO:40, SEQ ID NO:41, SEQ ID NO:43, SEQ ID NO:44, SEQ ID NO:46, SEQ ID NO:47, SEQ ID NO:49, SEQ ID NO:50, SEQ ID NO:52, SEQ ID NO:53, SEQ ID NO:55, SEQ ID NO:56, SEQ ID NO:58, SEQ ID NO:59, SEQ ID NO:61, SEQ ID NO:62, SEQ ID NO:64, SEQ ID NO:65, SEQ ID NO:67, SEQ ID NO:68, SEQ ID NO:70, SEQ ID NO:71, SEQ ID NO:72, SEQ ID NO:73, SEQ ID NO:75, SEQ ID NO:76, and/or SEQ ID NO:77, but which, due to natural variations caused by, for example, mutation or recombination, has a similar but not identical sequence. Because natural selection typically selects against alterations that affect function, allelic variants (i.e. alleles corresponding to, or of, cited nucleic acid sequences) usually encode proteins having similar activity to that of the protein encoded by the gene to which they are being compared. Allelic variants of genes or nucleic acid molecules can also comprise alterations in the 5′ or 3′ untranslated regions of the gene (e.g., in regulatory control regions), or can involve alternative splicing of a nascent transcript, thereby bringing alternative exons into juxtaposition. Allelic variants are well known to those skilled in the art and would be expected to occur naturally within canids, since the genome is diploid, and sexual reproduction will result in the reassortment of alleles.

In one embodiment of the present invention, an isolated caTAg1 protein is encoded by a nucleic acid molecule that hybridizes under stringent hybridization conditions to a gene encoding a canine TAg1 protein. The minimal size of a caTAg1 protein of the present invention is a size sufficient to be encoded by a nucleic acid molecule capable of forming a stable hybrid (i.e., hybridizing under stringent hybridization conditions) with the complementary sequence of a nucleic acid molecule encoding the corresponding natural protein. The size of a nucleic acid molecule encoding such a protein is dependent on the nucleic acid composition and the percent homology between the canine TAg1 nucleic acid molecule and the complementary nucleic acid sequence. It can easily be understood that the extent of homology required to form a stable hybrid under stringent conditions can vary depending on whether the homologous sequences are interspersed throughout a given nucleic acid molecule or are clustered (i.e., localized) in distinct regions on a given nucleic acid molecule.

The minimal size of a nucleic acid molecule capable of forming a stable hybrid with a gene encoding a canine TAg1 protein is at least about 12 to about 15 nucleotides in length if the nucleic acid molecule is GC-rich and at least about 15 to about 17 bases in length if it is AT-rich. The minimal size of a nucleic acid molecule used to encode a TAg1 protein homologue of the present invention is from about 12 to about 18 nucleotides in length. Thus, the minimal size of a TAg1 protein homologue of the present invention is from about 4 to about 6 amino acids in length. There is no limit, other than a practical limit, on the maximal size of a nucleic acid molecule encoding a canine TAg1 protein of the present invention because a nucleic acid molecule of the present invention can include a portion of a gene or cDNA or RNA, an entire gene or cDNA or RNA, or multiple genes or cDNA or RNA. The preferred size of a protein encoded by a nucleic acid molecule of the present invention depends on whether a full-length, fusion, multivalent, or functional portion of such a protein is desired.

Stringent hybridization conditions are determined based on defined physical properties of the caTAg1 gene, or other nucleic acid molecule, to which the nucleic acid molecule is being hybridized, and can be defined mathematically. Stringent hybridization conditions are those experimental parameters that allow an individual skilled in the art to identify significant similarities between heterologous nucleic acid molecules. These conditions are well known to those skilled in the art. See, for example, Sambrook, et al., 1989, Molecular Cloning: A Laboratory Manual, Cold Spring Harbor Labs Press, and Meinkoth, et al., 1984, Anal. Biochem. 138, 267-284, each of which is incorporated by reference herein in its entirety. As explained in detail in the cited references, the determination of hybridization conditions involves the manipulation of a set of variables including the ionic strength (M, in moles/liter), the hybridization temperature (° C.), the concentration of nucleic acid helix destabilizing agents (such as formamide), the average length of the shortest hybrid duplex (n), and the percent G+C composition of the fragment to which an unknown nucleic acid molecule is being hybridized. For nucleic acid molecules of at least about 150 nucleotides, these variables are inserted into a standard mathematical formula to calculate the melting temperature, or Tₘ, of a given nucleic acid molecule. As defined in the formula below, Tₘ is the temperature at which two complementary nucleic acid molecule strands will disassociate, assuming 100% complementarity between the two strands:

Tₘ=81.5° C.+16.6 log M+0.41(%G+C)−500/n−0.61(% formamide).

For nucleic acid molecules smaller than about 50 nucleotides, hybrid stability is defined by the dissociation temperature (T_{d}), which is defined as the temperature at which 50% of the duplexes dissociate. For these smaller molecules, the stability at a standard ionic strength is defined by the following equation:

T_{d}=4(G+C)+2(A+T).

A temperature of 5° C. below T_{d} is used to detect hybridization between perfectly matched molecules.

Also well known to those skilled in the art is how base-pair mismatch, i.e. differences between two nucleic acid molecules being compared, including non-complementarity of bases at a given location, and gaps due to insertion or deletion of one or more bases at a given location on either of the nucleic acid molecules being compared, will affect Tₘ or T_{d} for nucleic acid molecules of different sizes. For example, Tₘ decreases about 1° C. for each 1% of mismatched base-pairs for hybrids greater than about 150 bp, and T_{d} decreases about 5° C. for each mismatched base-pair for hybrids below about 50 bp. Conditions for hybrids between about 50 and about 150 base-pairs can be determined empirically and without undue experimentation using standard laboratory procedures well known to those skilled in the art. These simple procedures allow one skilled in the art to set the hybridization conditions (by altering, for example, the salt concentration, the formamide concentration or the temperature) so that only nucleic acid hybrids with less than a specified % base-pair mismatch will hybridize. Stringent hybridization conditions are commonly understood by those skilled in the art to be those experimental conditions that will allow hybridization between molecules having about 30% or less base-pair mismatch (i.e., about 70% or greater identity). Because one skilled in the art can easily determine whether a given nucleic acid molecule to be tested is less than or greater than about 50 nucleotides, and can therefore choose the appropriate formula for determining hybridization conditions, he or she can determine whether the nucleic acid molecule will hybridize with a given gene under stringent hybridization conditions and similarly whether the nucleic acid molecule will hybridize under conditions designed to allow a desired amount of base pair mismatch.

Hybridization reactions are often carried out by attaching the nucleic acid molecule to be hybridized to a solid support such as a membrane, and then hybridizing with a labeled nucleic acid molecule, typically referred to as a probe, suspended in a hybridization solution. Examples of common hybridization reaction techniques include, but are not limited to, the well-known Southern and northern blotting procedures. Typically, the actual hybridization reaction is done under non-stringent conditions, i.e., at a lower temperature and/or a higher salt concentration, and then high stringency is achieved by washing the membrane in a solution with a higher temperature and/or lower salt concentration in order to achieve the desired stringency.

For example, if the skilled artisan wished to identify a nucleic acid molecule that hybridizes under stringent hybridization conditions with a canine nucleic acid molecule of about 150 bp in length, the following conditions could preferably be used. The average G+C content of canine DNA is about 52%. The unknown nucleic acid molecules would be attached to a support membrane, and the 150 bp probe would be labeled, e.g. with a radioactive tag. The hybridization reaction could be carried out in a solution comprising 2×SSC in the absence of nucleic acid helix destabilizing agents, at a temperature of about 37° C. (low stringency conditions). Solutions of differing concentrations of SSC can be made by one of skill in the art by diluting a stock solution of 20×SSC (175.3 gram NaCl and about 88.2 gram sodium citrate in 1 liter of water, pH 7) to obtain the desired concentration of SSC. In order to achieve high stringency hybridization, the skilled artisan would calculate the washing conditions required to allow up to 30% base-pair mismatch. For example, in a wash solution comprising 1×SSC in the absence of nucleic acid helix destabilizing agents, the Tₘ of perfect hybrids would be about 86° C.:

81.5° C.+16.6 log (0.15M)+(0.41×52)−(500/150)−(0.61×0)=86° C.

Thus, to achieve hybridization with nucleic acid molecules having about 30% base-pair mismatch, hybridization washes would be carried out at a temperature of about 56° C. It is thus within the skill of one in the art to calculate additional hybridization temperatures based on the desired percentage base-pair mismatch, formulae and G/C content disclosed herein. For example, it is appreciated by one skilled in the art that as the nucleic acid molecule to be tested for hybridization against nucleic acid molecules of the present invention having sequences specified herein becomes longer than 150 nucleotides, the Tₘ for a hybridization reaction allowing up to 30% base-pair mismatch will not vary significantly from 56° C.

Furthermore, it is known in the art that there are commercially available computer programs for determining the degree of similarity between two nucleic acid or protein sequences. These computer programs include various known methods to determine the percentage identity and the number and length of gaps between hybrid nucleic acid molecules or proteins. Preferred methods to determine the percent identity among amino acid sequences and also among nucleic acid sequences include analysis using one or more of the commercially available computer programs designed to compare and analyze nucleic acid or amino acid sequences. These computer programs include, but are not limited to, the SeqLab® Wisconsin Package™ Version 10.0-UNIX sequence analysis software, available from Genetics Computer Group, Madison, Wis.; and DNAsis® sequence analysis software, version 2.0, available from Hitachi Software, San Bruno, Calif. Such software programs represent a collection of algorithms paired with a graphical user interface for using the algorithms. The DNAsis version 2.0 software and SeqLab Wisconsin Package Version 10.0-UNIX software, for example, employ a particular algorithm, the Needleman-Wunsch algorithm to perform pair-wise comparisons between two sequences to yield a percentage identity score, see Needleman, S. B. and Wunch, C. D., 1970, J. Mol. Biol., 48, 443, which is incorporated herein by reference in its entirety. Such algorithms, including the Needleman-Wunsch algorithm, are commonly used by those skilled in the nucleic acid and amino acid sequencing art to compare sequences. A preferred method to determine percent identity among amino acid sequences and also among nucleic acid sequences includes using the Needleman-Wunsch algorithm, available in the SeqLab Wisconsin Package Version 10.0-UNIX software (hereinafter “SeqLab”), using the Pairwise Comparison/Gap function with the nwsgapdna.cmp scoring matrix, the gap creation penalty and the gap extension penalties set at default values, and the gap shift limits set at maximum (hereinafter referred to as “SeqLab default parameters”). An additional preferred method to determine percent identity among amino acid sequences and also among nucleic acid sequences includes using the Higgins-Sharp algorithm, available in the DNAsis version 2.0 software (hereinafter “DNAsis”), with the gap penalty set at 5, the number of top diagonals set at 5, the fixed gap penalty set at 10, the k-tuple set at 2, the window size set at 5, and the floating gap penalty set at 10. A particularly preferred method to determine percent identity among amino acid sequences and also among nucleic acid sequences includes using the Needleman-Wunsch algorithm available in the DNAsis version 2.0 software, using the GCG default parameter function.

A preferred canine TAg1 protein includes an isolated protein selected from the group consisting of: (a) a protein comprising an amino acid sequence selected from the group consisting of SEQ ID NO:3, SEQ ID NO:9, SEQ ID NO:12, SEQ ID NO:15, SEQ ID NO:18, SEQ ID NO:24, SEQ ID NO:27, SEQ ID NO:30, SEQ ID NO:33, SEQ ID NO:36, SEQ ID NO:39, SEQ ID NO:42, SEQ ID NO:45, SEQ ID NO:48, SEQ ID NO:51, SEQ ID NO:54, SEQ ID NO:57, SEQ ID NO:60, SEQ ID NO:63, SEQ ID NO:66, SEQ ID NO:69, and/or SEQ ID NO:74; (b) a protein comprising an at least 6 consecutive amino acid portion identical in sequence to an at least 6 consecutive amino acid portion of a sequence selected from the group consisting of SEQ ID NO:9, SEQ ID NO:24, SEQ ID NO:33, and SEQ ID NO:36; (c) a protein comprising an at least 15 consecutive amino acid portion identical in sequence to an at least 15 consecutive amino acid portion of a sequence selected from the group consisting of SEQ ID NO:12, SEQ ID NO:27, SEQ ID NO:39, and SEQ ID NO:42 and (d) a protein comprising an at least 42 consecutive amino acid portion identical in sequence to an at least 42 consecutive amino acid portion of a sequence selected from the group consisting of SEQ ID NO:3, SEQ ID NO:15, SEQ ID NO:18, SEQ ID NO:30, SEQ ID NO:45, SEQ ID NO:48, SEQ ID NO:51, SEQ ID NO:54, SEQ ID NO:57, SEQ ID NO:60, SEQ ID NO:63, SEQ ID NO:66, SEQ ID NO:69, and SEQ ID NO:74.

One embodiment of the present invention includes canine TAg1 proteins. A preferred canine TAg1 protein includes a protein encoded by a nucleic acid molecule which is less than about 50 nucleotides and which hybridizes under conditions which preferably allow about 30% base pair mismatch, more preferably under conditions which allow about 25% base pair mismatch, more preferably under conditions which allow about 20% base pair mismatch, more preferably under conditions which allow about 15% base pair mismatch, more preferably under conditions which allow about 10% base pair mismatch and even more preferably under conditions which allow about 5% base pair mismatch with the complement of a nucleic acid molecule selected from the group consisting of SEQ ID NO:2, SEQ ID NO:5, SEQ ID NO:8, SEQ ID NO:11, SEQ ID NO:14, SEQ ID NO:17, SEQ ID NO:20, SEQ ID NO:23, SEQ ID NO:26, SEQ ID NO:29, SEQ ID NO:32, SEQ ID NO:35, SEQ ID NO:38, SEQ ID NO:41, SEQ ID NO:44, SEQ ID NO:47, SEQ ID NO:50, SEQ ID NO:53, SEQ ID NO:56, SEQ ID NO:59, SEQ ID NO:62, SEQ ID NO:65, SEQ ID NO:68, SEQ ID NO:71, SEQ ID NO:73, and/or SEQ ID NO:76.

Another preferred canine TAg1 protein of the present invention includes a protein encoded by a nucleic acid molecule which is at least about 50 to at least about 150 nucleotides in length and which hybridizes under conditions which preferably allow about 30% base pair mismatch, more preferably under conditions which allow about 25% base pair mismatch, more preferably under conditions which allow about 20% base pair mismatch, more preferably under conditions which allow about 15% base pair mismatch, more preferably under conditions which allow about 10% base pair mismatch and even more preferably under conditions which allow about 5% base pair mismatch with a nucleic acid molecule selected from the group consisting of SEQ ID NO:2, SEQ ID NO:5, SEQ ID NO:8, SEQ ID NO:11, SEQ ID NO:14, SEQ ID NO:17, SEQ ID NO:20, SEQ ID NO:23, SEQ ID NO:26, SEQ ID NO:29, SEQ ID NO:32, SEQ ID NO:35, SEQ ID NO:38, SEQ ID NO:41, SEQ ID NO:44, SEQ ID NO:47, SEQ ID NO:50, SEQ ID NO:53, SEQ ID NO:56, SEQ ID NO:59, SEQ ID NO:62, SEQ ID NO:65, SEQ ID NO:68, SEQ ID NO:71, SEQ ID NO:73, and/or SEQ ID NO:76.

Another preferred canine TAg1 protein of the present invention includes a protein encoded by a nucleic acid molecule which is at least about 150 nucleotides and which hybridizes under conditions which preferably allow about 30% base pair mismatch, more preferably under conditions which allow about 25% base pair mismatch, more preferably under conditions which allow about 20% base pair mismatch, more preferably under conditions which allow about 15% base pair mismatch, more preferably under conditions which allow about 10% base pair mismatch and even more preferably under conditions which allow about 5% base pair mismatch with the complement of a nucleic acid molecule selected from the group consisting of SEQ ID NO:2, SEQ ID NO:5, SEQ ID NO:8, SEQ ID NO:11, SEQ ID NO:14, SEQ ID NO:17, SEQ ID NO:20, SEQ ID NO:23, SEQ ID NO:26, SEQ ID NO:29, SEQ ID NO:32, SEQ ID NO:35, SEQ ID NO:38, SEQ ID NO:41, SEQ ID NO:44, SEQ ID NO:47, SEQ ID NO:50, SEQ ID NO:53, SEQ ID NO:56, SEQ ID NO:59, SEQ ID NO:62, SEQ ID NO:65, SEQ ID NO:68, SEQ ID NO:71, SEQ ID NO:73, and/or SEQ ID NO:76.

Another embodiment of the present invention includes a canine TAg1 protein encoded by a nucleic acid molecule which hybridizes to an isolated nucleic acid molecule selected from the group consisting of SEQ ID NO:8, SEQ ID NO:10, SEQ ID NO:23, SEQ ID NO:25, SEQ ID NO:32, SEQ ID NO:34, SEQ ID NO:35, and SEQ ID NO:37, under conditions comprising (a) hybridizing in a solution comprising 1×SSC in the absence of nucleic acid helix destabilizing agents, at a temperature of about 37° C. and (b) washing in a solution comprising 1×SSC in the absence of nucleic acid helix destabilizing agents, at a temperature of about 56° C.

Yet another preferred canine TAg1 protein of the present invention includes a protein encoded by a nucleic acid molecule which is preferably about 70% identical, more preferably about 75% identical, more preferably about 80% identical, more preferably about 85% identical, more preferably about 90% identical and even more preferably about 95% identical to a nucleic acid molecule having the nucleic acid sequence SEQ ID NO:2, SEQ ID NO:5, SEQ ID NO:8, SEQ ID NO:11, SEQ ID NO:14, SEQ ID NO:17, SEQ ID NO:20, SEQ ID NO:23, SEQ ID NO:26, SEQ ID NO:29, SEQ ID NO:32, SEQ ID NO:35, SEQ ID NO:38, SEQ ID NO:41, SEQ ID NO:44, SEQ ID NO:47, SEQ ID NO:50, SEQ ID NO:53, SEQ ID NO:56, SEQ ID NO:59, SEQ ID NO:62, SEQ ID NO:65, SEQ ID NO:68, SEQ ID NO:71, SEQ ID NO:73, and/or SEQ ID NO:76. Percent identity as used herein is determined using the Needleman-Wunsch algorithm available in the DNAsis version 2.0 software, using the GCG default parameter function.

Preferred canine proteins of the present invention include proteins having the amino acid sequence SEQ ID NO:3, SEQ ID NO:9, SEQ ID NO:12, SEQ ID NO:15, SEQ ID NO:18, SEQ ID NO:24, SEQ ID NO:27, SEQ ID NO:30, SEQ ID NO:33, SEQ ID NO:36, SEQ ID NO:39, SEQ ID NO:42, SEQ ID NO:45, SEQ ID NO:48, SEQ ID NO:51, SEQ ID NO:54, SEQ ID NO:57, SEQ ID NO:60, SEQ ID NO:63, SEQ ID NO:66, SEQ ID NO:69, and/or SEQ ID NO:74, including, but not limited to, the proteins consisting of amino acid sequence SEQ ID NO:3, SEQ ID NO:9, SEQ ID NO:12, SEQ ID NO:15, SEQ ID NO:18, SEQ ID NO:24, SEQ ID NO:27, SEQ ID NO:30, SEQ ID NO:33, SEQ ID NO:36, SEQ ID NO:39, SEQ ID NO:42, SEQ ID NO:45, SEQ ID NO:48, SEQ ID NO:51, SEQ ID NO:54, SEQ ID NO:57, SEQ ID NO:60, SEQ ID NO:63, SEQ ID NO:66, SEQ ID NO:69, and/or SEQ ID NO:74, fusion proteins and multivalent proteins. Preferred canine proteins of the present invention include proteins comprising homologues of a protein having the amino acid sequence SEQ ID NO:3, SEQ ID NO:9, SEQ ID NO:12, SEQ ID NO:15, SEQ ID NO:18, SEQ ID NO:24, SEQ ID NO:27, SEQ ID NO:30, SEQ ID NO:33, SEQ ID NO:36, SEQ ID NO:39, SEQ ID NO:42, SEQ ID NO:45, SEQ ID NO:48, SEQ ID NO:51, SEQ ID NO:54, SEQ ID NO:57, SEQ ID NO:60, SEQ ID NO:63, SEQ ID NO:66, SEQ ID NO:69, and/or SEQ ID NO:74 wherein such a homologue comprises at least one epitope that elicits an immune response against a protein having an amino acid sequence SEQ ID NO:3, SEQ ID NO:9, SEQ ID NO:12, SEQ ID NO:15, SEQ ID NO:18, SEQ ID NO:24, SEQ ID NO:27, SEQ ID NO:30, SEQ ID NO:33, SEQ ID NO:36, SEQ ID NO:39, SEQ ID NO:42, SEQ ID NO:45, SEQ ID NO:48, SEQ ID NO:51, SEQ ID NO:54, SEQ ID NO:57, SEQ ID NO:60, SEQ ID NO:63, SEQ ID NO:66, SEQ ID NO:69, and/or SEQ ID NO:74. Also preferred are proteins having sequences described herein from which the initiating methionine has been removed. Likewise, also preferred are proteins encoded by nucleic acid molecules having nucleic acid sequence SEQ ID NO:2, SEQ ID NO:5, SEQ ID NO:8, SEQ ID NO:11, SEQ ID NO:14, SEQ ID NO:17, SEQ ID NO:20, SEQ ID NO:23, SEQ ID NO:26, SEQ ID NO:29, SEQ ID NO:32, SEQ ID NO:35, SEQ ID NO:38, SEQ ID NO:41, SEQ ID NO:44, SEQ ID NO:47, SEQ ID NO:50, SEQ ID NO:53, SEQ ID NO:56, SEQ ID NO:59, SEQ ID NO:62, SEQ ID NO:65, SEQ ID NO:68, SEQ ID NO:71, SEQ ID NO:73, and/or SEQ ID NO:76, or by homologues thereof.

A preferred isolated protein of the present invention is a protein encoded by at least one of the following nucleic acid molecules: ncapTAg1₁₂₃₆, ncapTAg1₉₇₈, ncapTAg1₁₄₄, ncapTAg1₂₂₂, ncapTAg1₆₁₂, ncasTAg1₁₂₆₉, ncasTAg1₉₈₇, ncasTAg1₁₅₃, ncasTAg1₂₂₂, ncasTAg1₆₁₂, rncasTAg1₉₈₇, ncasTAg1₆₈₁, and/or rncasTAg1₆₈₁, or allelic variants of any of these nucleic acid molecules. Also preferred is an isolated protein encoded by a nucleic acid molecule having nucleic acid sequence SEQ ID NO:2, SEQ ID NO:5, SEQ ID NO:8, SEQ ID NO:11, SEQ ID NO:14, SEQ ID NO:17, SEQ ID NO:20, SEQ ID NO:23, SEQ ID NO:26, SEQ ID NO:29, SEQ ID NO:32, SEQ ID NO:35, SEQ ID NO:38, SEQ ID NO:41, SEQ ID NO:44, SEQ ID NO:47, SEQ ID NO:50, SEQ ID NO:53, SEQ ID NO:56, SEQ ID NO:59, SEQ ID NO:62, SEQ ID NO:65, SEQ ID NO:68, SEQ ID NO:71, SEQ ID NO:73, and/or SEQ ID NO:76; or a protein encoded by an allelic variant of any of these listed nucleic acid molecules.

Translation of SEQ ID NO:2, the coding strand of nncapTAg1₁₂₃₆, yields a protein of about 326 amino acids, denoted herein as PcapTg1₃₂₆, the amino acid sequence of which is presented in SEQ ID NO:3, assuming a first in-frame codon extending from nucleotide 34 to nucleotide 36 of SEQ ID NO:2 and a last in-frame codon extending from nucleotide 1012 to nucleotide 1014 of SEQ ID NO:2.

Translation of SEQ ID NO:17, the coding strand of ncasTAg1₁₂₆₉, yields a protein of about 329 amino acids, denoted herein as PcasTAg1₃₂₉, the amino acid sequence of which is presented in SEQ ID NO:18, assuming a first in-frame codon extending from nucleotide 58 to nucleotide 60 of SEQ ID NO:17 and a last in-frame codon extending from nucleotide 1042 to nucleotide 1044 of SEQ ID NO:17.

Preferred TAg1 proteins of the present invention include proteins that are at least about 70%, more preferably at least about 75%, more preferably at least about 80%, more preferably at least about 85%, more preferably at least about 90%, more preferably at least about 92%, more preferably at least about 95%, even more preferably at least about 98%, and even more preferably about 100% identical to PcapTAg1₃₂₆ and/or PcasTAg1₃₂₉. More preferred are TAg1 proteins comprising PcapTAg1₃₂₆, PcapTAg1₄₈, PcapTAg1₇₄, PcapTAg1₂₀₄, PcasTAg1₃₂₉, PcasTAg1₅₁, PcasTAg1₇₄, PcasTAg1₂₀₄, and/or PcasTAg1₂₂₇; and proteins encoded by allelic variants of nucleic acid molecules encoding proteins PcapTAg1₃₂₆, PcapTAg1₄₈, PcapTAg1₇₄, PcapTAg1₂₀₄, PcasTAg1₃₂₉, PcasTAg1₅₁, PcasTAg1₇₄, PcasTAg1₂₀₄, and/or PcasTAg1₂₂₇. Percent identity as used herein is determined using the Needleman-Wunsch algorithm available in the DNAsis version 2.0 software, using the GCG default parameter function.

As such, preferred TAg1 proteins of the present invention include proteins having amino acid sequences that are at least about 70%, more preferably at least about 75%, more preferably at least about 80%, more preferably at least about 85%, more preferably at least about 90%, more preferably at least about 92%, more preferably at least about 95%, even more preferably at least about 98%, and even more preferably about 100% identical to amino acid sequence SEQ ID NO:3, SEQ ID NO:9, SEQ ID NO:12, SEQ ID NO:15, SEQ ID NO:18, SEQ ID NO:24, SEQ ID NO:27, SEQ ID NO:30, SEQ ID NO:33, SEQ ID NO:36, SEQ ID NO:39, SEQ ID NO:42, SEQ ID NO:45, SEQ ID NO:48, SEQ ID NO:51, SEQ ID NO:54, SEQ ID NO:57, SEQ ID NO:60, SEQ ID NO:63, SEQ ID NO:66, SEQ ID NO:69, and/or SEQ ID NO:74. Also preferred are fragments of any of such proteins. Percent identity as used herein is determined using the Needleman-Wunsch algorithm available in the DNAsis version 2.0 software, using the GCG default parameter function. More preferred are TAg1 proteins comprising amino acid sequences SEQ ID NO:3, SEQ ID NO:9, SEQ ID NO:12, SEQ ID NO:15, SEQ ID NO:18, SEQ ID NO:24, SEQ ID NO:27, SEQ ID NO:30, SEQ ID NO:33, SEQ ID NO:36, SEQ ID NO:39, SEQ ID NO:42, SEQ ID NO:45, SEQ ID NO:48, SEQ ID NO:51, SEQ ID NO:54, SEQ ID NO:57, SEQ ID NO:60, SEQ ID NO:63, SEQ ID NO:66, SEQ ID NO:69, and/or SEQ ID NO:74; and TAg1 proteins encoded by allelic variants of nucleic acid molecules encoding TAg1 proteins having amino acid sequences SEQ ID NO:3, SEQ ID NO:9, SEQ ID NO:12, SEQ ID NO:15, SEQ ID NO:18, SEQ ID NO:24, SEQ ID NO:27, SEQ ID NO:30, SEQ ID NO:33, SEQ ID NO:36, SEQ ID NO:39, SEQ ID NO:42, SEQ ID NO:45, SEQ ID NO:48, SEQ ID NO:51, SEQ ID NO:54, SEQ ID NO:57, SEQ ID NO:60, SEQ ID NO:63, SEQ ID NO:66, SEQ ID NO:69, and/or SEQ ID NO:74.

In one embodiment, a preferred TAg1 protein comprises an amino acid sequence of at least about 5 amino acids in length, preferably at least about 10 amino acids in length, preferably at least about 15 amino acids in length, preferably at least about 20 amino acids in length, preferably at least about 25 amino acids in length, preferably at least about 30 amino acids in length, preferably at least about 35 amino acids in length, preferably at least about 50 amino acids in length, preferably at least about 60 amino acids in length, preferably at least about 75 amino acids in length, preferably at least about 100 amino acids in length, preferably at least about 200 amino acids in length, preferably at least about 250 amino acids in length, preferably at least about 300 amino acids in length, and preferably at least about 320 amino acids in length. Within this embodiment, a preferred canine TAg1 protein of the present invention has an amino acid sequence comprising at least a portion of SEQ ID NO:3 and/or SEQ ID NO:18. In one embodiment, a preferred canine TAg1 protein comprises a full-length protein, i.e., a protein encoded by a full-length coding region with or without an initiating methionine residue.

Preferred TAg1 proteins of the present invention include proteins encoded by nucleic acid molecules comprising at least a portion of ncapTAg1₁₂₃₆, ncapTAg1₉₇₈, ncapTAg1₁₄₄, ncapTAg1₂₂₂, ncapTAg1₆₁₂, ncasTAg1₁₂₆₉, ncasTAg1₉₈₇, ncasTAg1₁₅₃, ncasTAg1₂₂₂, ncasTAg1₆₁₂, rncasTAg1₉₈₇, ncasTAg1₆₈₁, and/or rncasTAg1₆₈₁, as well as TAg1 proteins encoded by allelic variants of such nucleic acid molecules.

Also preferred are TAg1 proteins encoded by nucleic acid molecules having nucleic acid sequences comprising at least a portion of SEQ ID NO:2, SEQ ID NO:5, SEQ ID NO:8, SEQ ID NO:11, SEQ ID NO:14, SEQ ID NO:17, SEQ ID NO:20, SEQ ID NO:23, SEQ ID NO:26, SEQ ID NO:29, SEQ ID NO:32, SEQ ID NO:35, SEQ ID NO:38, SEQ ID NO:41, SEQ ID NO:44, SEQ ID NO:47, SEQ ID NO:50, SEQ ID NO:53, SEQ ID NO:56, SEQ ID NO:59, SEQ ID NO:62, SEQ ID NO:65, SEQ ID NO:68, SEQ ID NO:71, SEQ ID NO:73, and/or SEQ ID NO:76, as well as allelic variants of these nucleic acid molecules.

In another embodiment, a preferred canine TAg1 protein of the present invention is encoded by a nucleic acid molecule comprising at least about 15 nucleotides, preferably at least about 20 nucleotides, preferably at least about 25 nucleotides, preferably at least about 35 nucleotides, at least about 50 nucleotides, at least about 60 nucleotides, at least about 75 nucleotides, preferably at least about 100 nucleotides, preferably at least about 250 nucleotides, preferably at least about 300 nucleotides, preferably at least about 350 nucleotides, preferably at least about 500 nucleotides, preferably at least about 750 nucleotides, preferably at least about 900 nucleotides, preferably at least about 950 nucleotides, preferably at least about 1000 nucleotides, and preferably at least about 1100 nucleotides. Within this embodiment is a TAg1 protein encoded by at least a portion ncapTAg1₁₂₃₆ and/or ncasTAg1₁₂₆₉ or by an allelic variant of either of these nucleic acid molecules. In one embodiment, a preferred canine TAg1 protein of the present invention is encoded by a nucleic acid molecule comprising an apparently full-length canine TAg1 coding region, i.e., a nucleic acid molecule encoding an apparently full-length canine TAg1 protein, with or without an initiating methionine amino acid residue.

A preferred canine TAg1 protein of the present invention is a compound that when administered to an animal in an effective manner, is capable of protecting that animal from disease. In accordance with the present invention, the ability of a TAg1 protein of the present invention to protect an animal from disease refers to the ability of that protein to, for example, treat, ameliorate and/or prevent disease.

One embodiment of a canine TAg1 protein of the present invention is a fusion protein that includes a canine TAg1 protein-containing domain attached to one or more fusion segments. Suitable fusion segments for use with the present invention include, but are not limited to, segments that can: enhance a protein's stability; act as an immunopotentiator; and/or assist in purification of a canine TAg1 protein (e.g., by affinity chromatography). A suitable fusion segment can be a domain of any size that has the desired function (e.g., imparts increased stability, imparts increased immunogenicity to a protein, and/or simplifies purification of a protein). Fusion segments can be joined to amino and/or carboxyl termini of the canine TAg1-containing domain of the protein and can be susceptible to cleavage in order to enable straight-forward recovery of a canine TAg1 protein. Fusion proteins are preferably produced by culturing a recombinant cell transformed with a fusion nucleic acid molecule that encodes a protein including the fusion segment attached to either the carboxyl and/or amino terminal end of a TAg1-containing domain. Preferred fusion segments include a metal binding domain (e.g., a poly-histidine segment); an immunoglobulin binding domain (e.g., Protein A; Protein G; T cell; B cell; Fc receptor or complement protein antibody-binding domains); a sugar binding domain (e.g., a maltose binding domain); and/or a “tag” domain (e.g., at least a portion of β-galactosidase, a strep tag peptide, a T7 tag peptide, a Flag™ peptide, or other domains that can be purified using compounds that bind to the domain, such as monoclonal antibodies). More preferred fusion segments include metal binding domains, such as a poly-histidine segment; a maltose binding domain; a strep tag peptide, such as that available from Biometra in Tampa, Fla.; and an S10 peptide.

In another embodiment, a canine TAg1 protein of the present invention also includes at least one additional protein segment that is capable of protecting an animal from one or more diseases. Such a multivalent protective protein can be produced by culturing a cell transformed with a nucleic acid molecule comprising two or more nucleic acid domains joined together in such a manner that the resulting nucleic acid molecule is expressed as a multivalent protective compound containing at least two protective compounds capable of protecting an animal from disease.

The present invention also includes mimetopes of canine TAg1 proteins of the present invention. As used herein, a mimetope of a canine TAg1 protein of the present invention refers to any compound that is able to mimic the activity of such a TAg1 protein, often because the mimetope has a structure that mimics the particular TAg1 protein. Mimetopes can be, but are not limited to: peptides that have been modified to decrease their susceptibility to degradation such as all-D retro peptides; anti-idiotypic and/or catalytic antibodies, or fragments thereof; non-proteinaceous immunogenic portions of an isolated protein (e.g., carbohydrate structures); and synthetic or natural organic molecules, including nucleic acids. Such mimetopes can be designed using computer-generated structures of proteins of the present invention. Mimetopes can also be obtained by generating random samples of molecules, such as oligonucleotides, peptides or other organic molecules, and screening such samples by affinity chromatography techniques using the corresponding binding partner.

Another embodiment of the present invention is an isolated nucleic acid molecule comprising a canine TAg1 nucleic acid molecule. The identifying characteristics of such nucleic acid molecules are heretofore described. A nucleic acid molecule of the present invention can include an isolated natural canine TAg1 gene or a homologue thereof, the latter of which is described in more detail below. A nucleic acid molecule of the present invention can include one or more regulatory regions, full-length or partial coding regions, or combinations thereof. The minimal size of a nucleic acid molecule of the present invention is a size sufficient to allow the formation of a stable hybrid (i.e., hybridization under stringent hybridization conditions) with the complementary sequence of another nucleic acid molecule. As such, the minimal size of a TAg1 nucleic acid molecule of the present invention is from about 12 to about 18 nucleotides in length. Suitable and preferred canids from which to isolate nucleic acid molecules of the present invention are disclosed herein. Preferred TAg1 nucleic acid molecules include Canis familiaris nucleic acid molecules.

In accordance with the present invention, an isolated nucleic acid molecule is a nucleic acid molecule that has been removed from its natural milieu (i.e., that has been subjected to human manipulation) and can include DNA, RNA, or derivatives of either DNA or RNA, genomic DNA, cDNA, mRNA. A nucleic acid molecule of the present invention can be double or single-stranded. As such, “isolated” does not reflect the extent to which the nucleic acid molecule has been purified. An isolated canine TAg1 nucleic acid molecule of the present invention, can be isolated from its natural source or produced using recombinant DNA technology (e.g., polymerase chain reaction (PCR) amplification or cloning) or chemical synthesis. Isolated canine TAg1 nucleic acid molecules, and homologues thereof, can include, for example, natural allelic variants and nucleic acid molecules modified by nucleotide insertions, deletions, substitutions, and/or inversions in a manner such that the modifications do not substantially interfere with the nucleic acid molecule's ability to encode a TAg1 protein of the present invention.

A canine TAg1 nucleic acid molecule homologue can be produced using a number of methods known to those skilled in the art, see, for example, Sambrook et al., 1989, Molecular Cloning: A Laboratory Manual, Cold Spring Harbor Labs Press; Sambrook et al., ibid., is incorporated by reference herein in its entirety. For example, nucleic acid molecules can be modified using a variety of techniques including, but not limited to, classic mutagenesis and recombinant DNA techniques such as site-directed mutagenesis, chemical treatment, restriction enzyme cleavage, ligation of nucleic acid fragments, PCR amplification, synthesis of oligonucleotide mixtures and ligation of mixture groups to “build” a mixture of nucleic acid molecules, and combinations thereof. Nucleic acid molecule homologues can be selected by hybridization with a canine TAg1 nucleic acid molecule or by screening for the function of a protein encoded by the nucleic acid molecule (e.g., ability to elicit an immune response against at least one epitope of a canine TAg1 protein or ability of the protein to bind to serum from an immune dog).

An isolated nucleic acid molecule of the present invention can include a nucleic acid sequence that encodes at least one canine TAg1 protein of the present invention, examples of such proteins being disclosed herein. Although the phrase “nucleic acid molecule” primarily refers to the physical nucleic acid molecule and the phrase “nucleic acid sequence” primarily refers to the sequence of nucleotides on the nucleic acid molecule, the two phrases can be used interchangeably, especially with respect to a nucleic acid molecule, or a nucleic acid sequence, being capable of encoding a canine TAg1 protein or being capable of hybridizing to a nucleic acid molecule or nucleic acid sequence.

A preferred nucleic acid molecule of the present invention, when administered to an animal, is capable of protecting that animal from disease. As will be disclosed in more detail below, such a nucleic acid molecule can be, or encode, an antisense RNA, a molecule capable of triple helix formation, a ribozyme, or other nucleic acid-based drug compound. In additional embodiments, a nucleic acid molecule of the present invention can encode a protective protein (e.g., a TAg1 protein of the present invention), the nucleic acid molecule being delivered to the animal, for example, by direct injection (i.e, as a genetic vaccine) or in a vehicle such as a recombinant virus vaccine or a recombinant cell vaccine.

A preferred canine TAg1 nucleic acid molecule of the present invention includes an isolated nucleic acid molecule selected from the group consisting of: (a) a nucleic acid molecule comprising a nucleic acid sequence selected from the group consisting of SEQ ID NO:2, SEQ ID NO:4, SEQ ID NO:5, SEQ ID NO:7, SEQ ID NO:8, SEQ ID NO:10, SEQ ID NO:11, SEQ ID NO:13, SEQ ID NO:14, SEQ ID NO:16, SEQ ID NO:17, SEQ ID NO:19, SEQ ID NO:20, SEQ ID NO:22, SEQ ID NO:23, SEQ ID NO:25, SEQ ID NO:26, SEQ ID NO:28, SEQ ID NO:29, SEQ ID NO:31, SEQ ID NO:32, SEQ ID NO:34, SEQ ID NO:35, SEQ ID NO:37, SEQ ID NO:38, SEQ ID NO:40, SEQ ID NO:41, SEQ ID NO:43, SEQ ID NO:44, SEQ ID NO:46, SEQ ID NO:47, SEQ ID NO:49, SEQ ID NO:50, SEQ ID NO:52, SEQ ID NO:53, SEQ ID NO:55, SEQ ID NO:56, SEQ ID NO:58, SEQ ID NO:59, SEQ ID NO:61, SEQ ID NO:62, SEQ ID NO:64, SEQ ID NO:65, SEQ ID NO:67, SEQ ID NO:68, SEQ ID NO:70, SEQ ID NO:71, SEQ ID NO:72, SEQ ID NO:73, SEQ ID NO:75, SEQ ID NO:76, and/or SEQ ID NO:77; and (b) a nucleic acid molecule comprising an at least 15 consecutive nucleotide portion identical in sequence to an at least 15 consecutive nucleotide portion of a nucleic acid sequence selected from the group consisting of: SEQ ID NO:8, SEQ ID NO:10, SEQ ID NO:23, SEQ ID NO:25, SEQ ID NO:32, SEQ ID NO:34, SEQ ID NO:35, and SEQ ID NO:37; (c) a nucleic acid molecule comprising an at least 20 consecutive nucleotide portion identical in sequence to an at least 20 consecutive nucleotide portion of a nucleic acid sequence selected from the group consisting of: SEQ ID NO:11, SEQ ID NO:13, SEQ ID NO:26, SEQ ID NO:28, SEQ ID NO:38, SEQ ID NO:40, SEQ ID NO:41, and SEQ ID NO:43; (d) a nucleic acid molecule comprising an at least 40 consecutive nucleotide portion identical in sequence to an at least 40 consecutive nucleotide portion of a nucleic acid sequence selected from the group consisting of: SEQ ID NO:2, SEQ ID NO:4, SEQ ID NO:5, SEQ ID NO:7, SEQ ID NO:14, SEQ ID NO:16, SEQ ID NO:17, SEQ ID NO:19, SEQ ID NO:20, SEQ ID NO:22, SEQ ID NO:29, SEQ ID NO:31, SEQ ID NO:44, SEQ ID NO:46, SEQ ID NO:47, SEQ ID NO:49, SEQ ID NO:50, SEQ ID NO:52, SEQ ID NO:53, SEQ ID NO:55, SEQ ID NO:56, SEQ ID NO:58, SEQ ID NO:59, SEQ ID NO:61, SEQ ID NO:62, SEQ ID NO:64, SEQ ID NO:65, SEQ ID NO:67, SEQ ID NO:68, SEQ ID NO:70, SEQ ID NO:71, SEQ ID NO:72, SEQ ID NO:73, SEQ ID NO:75, SEQ ID NO:76, and SEQ ID NO:77.

In one embodiment of the present invention, a preferred canine TAg1 nucleic acid molecule includes an isolated nucleic acid molecule which is less than about 50 nucleotides and which hybridizes under conditions which preferably allow about 30% base pair mismatch, more preferably under conditions which allow about 25% base pair mismatch, more preferably under conditions which allow about 20% base pair mismatch, more preferably under conditions which allow about 15% base pair mismatch, more preferably under conditions which allow about 10% base pair mismatch and even more preferably under conditions which allow about 5% base pair mismatch with a nucleic acid molecule selected from the group consisting of SEQ ID NO:2, SEQ ID NO:4, SEQ ID NO:5, SEQ ID NO:7, SEQ ID NO:8, SEQ ID NO:10, SEQ ID NO:11, SEQ ID NO:13, SEQ ID NO:14, SEQ ID NO:16, SEQ ID NO:17, SEQ ID NO:19, SEQ ID NO:20, SEQ ID NO:22, SEQ ID NO:23, SEQ ID NO:25, SEQ ID NO:26, SEQ ID NO:28, SEQ ID NO:29, SEQ ID NO:31, SEQ ID NO:32, SEQ ID NO:34, SEQ ID NO:35, SEQ ID NO:37, SEQ ID NO:38, SEQ ID NO:40, SEQ ID NO:41, SEQ ID NO:43, SEQ ID NO:44, SEQ ID NO:46, SEQ ID NO:47, SEQ ID NO:49, SEQ ID NO:50, SEQ ID NO:52, SEQ ID NO:53, SEQ ID NO:55, SEQ ID NO:56, SEQ ID NO:58, SEQ ID NO:59, SEQ ID NO:61, SEQ ID NO:62, SEQ ID NO:64, SEQ ID NO:65, SEQ ID NO:67, SEQ ID NO:68, SEQ ID NO:70, SEQ ID NO:71, SEQ ID NO:72, SEQ ID NO:73, SEQ ID NO:75, SEQ ID NO:76, and/or SEQ ID NO:77.

In one embodiment of the present invention, a preferred canine TAg1 nucleic acid molecule includes an isolated nucleic acid molecule which is at least about 50 nucleotides and less than about 150 nucleotides and which hybridizes under conditions which preferably allow about 30% base pair mismatch, more preferably under conditions which allow about 25% base pair mismatch, more preferably under conditions which allow about 20% base pair mismatch, more preferably under conditions which allow about 15% base pair mismatch, more preferably under conditions which allow about 10% base pair mismatch and even more preferably under conditions which allow about 5% base pair mismatch with a nucleic acid molecule selected from the group consisting of SEQ ID NO:2, SEQ ID NO:4, SEQ ID NO:5, SEQ ID NO:7, SEQ ID NO:8, SEQ ID NO:10, SEQ ID NO:11, SEQ ID NO:13, SEQ ID NO:14, SEQ ID NO:16, SEQ ID NO:17, SEQ ID NO:19, SEQ ID NO:20, SEQ ID NO:22, SEQ ID NO:23, SEQ ID NO:25, SEQ ID NO:26, SEQ ID NO:28, SEQ ID NO:29, SEQ ID NO:31, SEQ ID NO:32, SEQ ID NO:34, SEQ ID NO:35, SEQ ID NO:37, SEQ ID NO:38, SEQ ID NO:40, SEQ ID NO:41, SEQ ID NO:43, SEQ ID NO:44, SEQ ID NO:46, SEQ ID NO:47, SEQ ID NO:49, SEQ ID NO:50, SEQ ID NO:52, SEQ ID NO:53, SEQ ID NO:55, SEQ ID NO:56, SEQ ID NO:58, SEQ ID NO:59, SEQ ID NO:61, SEQ ID NO:62, SEQ ID NO:64, SEQ ID NO:65, SEQ ID NO:67, SEQ ID NO:68, SEQ ID NO:70, SEQ ID NO:71, SEQ ID NO:72, SEQ ID NO:73, SEQ ID NO:75, SEQ ID NO:76, and/or SEQ ID NO:77.

Another preferred canine TAg1 nucleic acid molecule of the present invention includes a nucleic acid molecule which is at least about 150 nucleotides and which hybridizes under conditions which preferably allow about 30% base pair mismatch, more preferably under conditions which allow about 25% base pair mismatch, more preferably under conditions which allow about 20% base pair mismatch, more preferably under conditions which allow about 15% base pair mismatch, more preferably under conditions which allow about 10% base pair mismatch and even more preferably under conditions which allow about 5% base pair mismatch with a nucleic acid molecule selected from the group consisting of SEQ ID NO:2, SEQ ID NO:4, SEQ ID NO:5, SEQ ID NO:7, SEQ ID NO:8, SEQ ID NO:10, SEQ ID NO:11, SEQ ID NO:13, SEQ ID NO:14, SEQ ID NO:16, SEQ ID NO:17, SEQ ID NO:19, SEQ ID NO:20, SEQ ID NO:22, SEQ ID NO:23, SEQ ID NO:25, SEQ ID NO:26, SEQ ID NO:28, SEQ ID NO:29, SEQ ID NO:31, SEQ ID NO:32, SEQ ID NO:34, SEQ ID NO:35, SEQ ID NO:37, SEQ ID NO:38, SEQ ID NO:40, SEQ ID NO:41, SEQ ID NO:43, SEQ ID NO:44, SEQ ID NO:46, SEQ ID NO:47, SEQ ID NO:49, SEQ ID NO:50, SEQ ID NO:52, SEQ ID NO:53, SEQ ID NO:55, SEQ ID NO:56, SEQ ID NO:58, SEQ ID NO:59, SEQ ID NO:61, SEQ ID NO:62, SEQ ID NO:64, SEQ ID NO:65, SEQ ID NO:67, SEQ ID NO:68, SEQ ID NO:70, SEQ ID NO:71, SEQ ID NO:72, SEQ ID NO:73, SEQ ID NO:75, SEQ ID NO:76, and/or SEQ ID NO:77.

Another embodiment of the present invention includes a nucleic acid molecule that hybridizes to an isolated nucleic acid molecule selected from the group consisting of SEQ ID NO:8, SEQ ID NO:10, SEQ ID NO:23, SEQ ID NO:25, SEQ ID NO:32, SEQ ID NO:34, SEQ ID NO:35, and SEQ ID NO:37, under conditions comprising (a) hybridizing in a solution comprising 1×SSC in the absence of nucleic acid helix destabilizing agents, at a temperature of about 37° C. and (b) washing in a solution comprising 1×SSC in the absence of nucleic acid helix destabilizing agents, at a temperature of about 56° C.

In one embodiment, a preferred canine TAg1 nucleic acid molecule of the present invention includes a nucleic acid molecule comprising a nucleic acid sequence that is preferably at least about 70%, more preferably at least about 75%, more preferably at least about 80%, more preferably at least about 85%, more preferably at least about 90%, more preferably at least about 95%, and even more preferably at least about 100% identical to a nucleic acid sequence selected from the group consisting of SEQ ID NO:2, SEQ ID NO:4, SEQ ID NO:5, SEQ ID NO:7, SEQ ID NO:8, SEQ ID NO:10, SEQ ID NO:11, SEQ ID NO:13, SEQ I NO:14, SEQ I NO:16, SEQ ID NO:17, SEQ ID NO:19, SEQ ID NO:20, SEQ ID NO:22, SEQ ID NO:23, SEQ ID NO:25, SEQ ID NO:26, SEQ ID NO:28, SEQ ID NO:29, SEQ ID NO:31, SEQ ID NO:32, SEQ ID NO:34, SEQ ID NO:35, SEQ ID NO:37, SEQ ID NO:38, SEQ ID NO:40, SEQ ID NO:41, SEQ ID NO:43, SEQ ID NO:44, SEQ ID NO:46, SEQ ID NO:47, SEQ ID NO:49, SEQ ID NO:50, SEQ ID NO:52, SEQ ID NO:53, SEQ ID NO:55, SEQ ID NO:56, SEQ ID NO:58, SEQ ID NO:59, SEQ ID NO:61, SEQ ID NO:62, SEQ ID NO:64, SEQ ID NO:65, SEQ ID NO:67, SEQ ID NO:68, SEQ ID NO:70, SEQ ID NO:71, SEQ ID NO:72, SEQ ID NO:73, SEQ ID NO:75, SEQ ID NO:76, and/or SEQ ID NO:77. Also preferred are fragments of such a nucleic acid molecule. A preferred fragment of a canine TAg1 nucleic acid molecules is at least about 15 nucleotides, preferably at least about 18 nucleotides, preferably at least about 20 nucleotides, preferably at least about 22 nucleotides, preferably at least about 24 nucleotides, preferably at least about 27 nucleotides, preferably at least about 30 nucleotides, preferably at least about 35 nucleotides, preferably at least about 40 nucleotides and even more preferably at least about 50 nucleotides. A particularly preferred canine nucleic acid molecule includes a Canis familiaris TAg1 nucleic acid molecule. Percent identity as used herein is determined using the Needleman-Wunsch algorithm available in the DNAsis version 2.0 software, using the GCG default parameter function.

In another embodiment, a preferred canine TAg1 nucleic acid molecule of the present invention comprises at least about 15 nucleotides, preferably at least about 20 nucleotides, preferably at least about 25 nucleotides, preferably at least about 35 nucleotides, at least about 50 nucleotides, at least about 60 nucleotides, at least about 75 nucleotides, preferably at least about 100 nucleotides, preferably at least about 250 nucleotides, preferably at least about 300 nucleotides, preferably at least about 350 nucleotides, preferably at least about 500 nucleotides, preferably at least about 750 nucleotides, preferably at least about 900 nucleotides, preferably at least about 950 nucleotides, preferably at least about 1000 nucleotides, and preferably at least about 1100 nucleotides. Within this embodiment is a TAg1 nucleic acid molecule comprising ncapTAg1₁₂₃₆ and/or ncasTAg1₁₂₆₉ or an allelic variant of either of these nucleic acid molecules. In one embodiment, a preferred canine TAg1 nucleic acid molecule comprises an apparently full-length canine TAg1 coding region, i.e., a nucleic acid molecule encoding an apparently full-length canine TAg1 protein, with or without an initiating methionine amino acid residue.

A fragment of a canine TAg1 nucleic acid molecule of the present invention preferably comprises at least about 15 nucleotides, more preferably at least about 18 nucleotides, more preferably at least about 20 nucleotides, more preferably at least about 25 nucleotides, more preferably at least about 30 nucleotides, more preferably at least about 35 nucleotides, more preferably at least about 40 nucleotides, more preferably at least about 45 nucleotides, more preferably at least about 50 nucleotides, more preferably at least about 60 nucleotides, more preferably at least about 70 nucleotides, more preferably at least about 80 nucleotides, more preferably at least about 90 nucleotides, and even more preferably at least about 100 contiguous nucleotides identical in sequence to a sequence selected from the group consisting of SEQ ID NO:2, SEQ ID NO:4, SEQ ID NO:5, SEQ ID NO:7, SEQ ID NO:8, SEQ ID NO:10, SEQ ID NO:11, SEQ ID NO:13, SEQ ID NO:14, SEQ ID NO:16, SEQ ID NO:17, SEQ ID NO:19, SEQ ID NO:20, SEQ ID NO:22, SEQ ID NO:23, SEQ ID NO:25, SEQ ID NO:26, SEQ ID NO:28, SEQ ID NO:29, SEQ ID NO:31, SEQ ID NO:32, SEQ ID NO:34, SEQ ID NO:35, SEQ ID NO:37, SEQ ID NO:38, SEQ ID NO:40, SEQ ID NO:41, SEQ ID NO:43, SEQ ID NO:44, SEQ ID NO:46, SEQ ID NO:47, SEQ ID NO:49, SEQ ID NO:50, SEQ ID NO:52, SEQ ID NO:53, SEQ ID NO:55, SEQ ID NO:56, SEQ ID NO:58, SEQ ID NO:59, SEQ ID NO:61, SEQ ID NO:62, SEQ ID NO:64, SEQ ID NO:65, SEQ ID NO:67, SEQ ID NO:68, SEQ ID NO:70, SEQ ID NO:71, SEQ ID NO:72, SEQ ID NO:73, SEQ ID NO:75, SEQ ID NO:76, and/or SEQ ID NO:77.

The phrase, a nucleic acid molecule comprising at least “x” contiguous, or consecutive nucleotides identical in sequence to at least “x” contiguous, or consecutive nucleotides of a nucleic acid molecule selected from the group consisting of SEQ ID NO:“y”, refers to an “x”-nucleotide in length nucleic acid molecule that is identical in sequence to an “x”-nucleotide portion of SEQ ID NO:“y”, as well as to nucleic acid molecules that are longer in length than “x”. The additional length may be in the form of nucleotides that extend from either the 5′ or the 3′ end(s) of the contiguous identical “x”-nucleotide portion. The 5′ and/or 3′ extensions can include one or more extensions that have no identity to a molecule of the present invention, as well as extensions that show similarity or identity to cited nucleic acids sequences or portions thereof.

One embodiment of the present invention is a nucleic acid molecule comprising all or part of nucleic acid molecules ncapTAg1₁₂₃₆, ncapTAg1₉₇₈, ncapTAg1₁₄₄, ncapTAg1₂₂₂, ncapTAg1₆₁₂, ncasTAg1₁₂₆₉, ncasTAg1₉₈₇, ncasTAg1₁₅₃, ncasTAg1₂₂₂, ncasTAg1₆₁₂, rncasTAg1₉₈₇, ncasTAg1₆₈₁, and/or rncasTAg1₆₈₁, or allelic variants of these nucleic acid molecules. As such, a preferred nucleic acid molecule of the present invention includes at least a portion of nucleic acid sequence SEQ ID NO:2, SEQ ID NO:4, SEQ ID NO:5, SEQ ID NO:7, SEQ ID NO:8, SEQ ID NO:10, SEQ ID NO:11, SEQ ID NO:13, SEQ ID NO:14, SEQ ID NO:16, SEQ ID NO:17, SEQ ID NO:19, SEQ ID NO:20, SEQ ID NO:22, SEQ ID NO:23, SEQ ID NO:25, SEQ ID NO:26, SEQ ID NO:28, SEQ ID NO:29, SEQ ID NO:31, SEQ ID NO:32, SEQ ID NO:34, SEQ ID NO:35, SEQ ID NO:37, SEQ ID NO:38, SEQ ID NO:40, SEQ ID NO:41, SEQ ID NO:43, SEQ ID NO:44, SEQ ID NO:46, SEQ ID NO:47, SEQ ID NO:49, SEQ ID NO:50, SEQ ID NO:52, SEQ ID NO:53, SEQ ID NO:55, SEQ ID NO:56, SEQ ID NO:58, SEQ ID NO:59, SEQ ID NO:61, SEQ ID NO:62, SEQ ID NO:64, SEQ ID NO:65, SEQ ID NO:67, SEQ ID NO:68, SEQ ID NO:70, SEQ ID NO:71, SEQ ID NO:72, SEQ ID NO:73, SEQ ID NO:75, SEQ ID NO:76, and/or SEQ ID NO:77, as well as allelic variants of nucleic acid molecules having these nucleic acid sequences and homologues of nucleic acid molecules having these nucleic acid sequences; preferably such a homologue encodes or is complementary to a nucleic acid molecule that encodes at least one epitope that elicits an immune response against a protein having an amino acid sequence SEQ ID NO:3, SEQ ID NO:9, SEQ ID NO:12, SEQ ID NO:15, SEQ ID NO:18, SEQ ID NO:24, SEQ ID NO:27, SEQ ID NO:30, SEQ ID NO:33, SEQ ID NO:36, SEQ ID NO:39, SEQ ID NO:42, SEQ ID NO:45, SEQ ID NO:48, SEQ ID NO:51, SEQ ID NO:54, SEQ ID NO:57, SEQ ID NO:60, SEQ ID NO:63, SEQ ID NO:66, SEQ ID NO:69, and/or SEQ ID NO:74. Such nucleic acid molecules can include nucleotides in addition to those included in the SEQ ID NOs, such as, but not limited to, a full-length gene, a full-length coding region, a nucleic acid molecule encoding a fusion protein, or a nucleic acid molecule encoding a multivalent protective compound.

In one embodiment, a TAg1 nucleic acid molecule of the present invention encodes a protein that is at least about 70%, more preferably at least about 75%, more preferably at least about 80%, more preferably at least about 85%, more preferably at least about 90%, more preferably at least about 92%, more preferably at least about 95%, even more preferably at least about 98%, and even more preferably about 100% identical to PcapTAg1₃₂₆ and/or PcasTAg1₃₂₉ or a fragment thereof. Even more preferred is a nucleic acid molecule encoding PcapTAg1₃₂₆, PcapTAg1₄₈, PcapTAg1₇₄, PcapTAg1₂₀₄, PcasTAg1₃₂₉, PcasTAg1₅₁, PcasTAg1₇₄, PcasTAg1₂₀₄, and/or PcasTAg1₂₂₇, and/or an allelic variant of such a nucleic acid molecule. Percent identity as used herein is determined using the Needleman-Wunsch algorithm available in the DNAsis version 2.0 software, using the GCG default parameter function.

A preferred TAg1 nucleic acid molecule of the present invention encodes a protein having an amino acid sequence that is at least about 70%, more preferably at least about 75%, more preferably at least about 80%, more preferably at least about 85%, more preferably at least about 90%, more preferably at least about 92%, more preferably at least about 95%, even more preferably at least about 98%, and even more preferably about 100% identical to SEQ ID NO:3, SEQ ID NO:9, SEQ ID NO:12, SEQ ID NO:15, SEQ ID NO:18, SEQ ID NO:24, SEQ ID NO:27, SEQ ID NO:30, SEQ ID NO:33, SEQ ID NO:36, SEQ ID NO:39, SEQ ID NO:42, SEQ ID NO:45, SEQ ID NO:48, SEQ ID NO:51, SEQ ID NO:54, SEQ ID NO:57, SEQ ID NO:60, SEQ ID NO:63, SEQ ID NO:66, SEQ ID NO:69, and/or SEQ ID NO:74 or a fragment of any of such amino acid sequences. Percent identity as used herein is determined using the Needleman-Wunsch algorithm available in the DNAsis version 2.0 software, using the GCG default parameter function.

The present invention also includes a TAg1 nucleic acid molecule encoding a protein having at least a portion of SEQ ID NO:3, SEQ ID NO:9, SEQ ID NO:12, SEQ ID NO:15, SEQ ID NO:18, SEQ ID NO:24, SEQ ID NO:27, SEQ ID NO:30, SEQ ID NO:33, SEQ ID NO:36, SEQ ID NO:39, SEQ ID NO:42, SEQ ID NO:45, SEQ ID NO:48, SEQ ID NO:51, SEQ ID NO:54, SEQ ID NO:57, SEQ ID NO:60, SEQ ID NO:63, SEQ ID NO:66, SEQ ID NO:69, and/or SEQ ID NO:74, as well as allelic variants of a TAg1 nucleic acid molecule encoding a protein having these sequences, including nucleic acid molecules that have been modified to accommodate codon usage properties of the cells in which such nucleic acid molecules are to be expressed.

In another embodiment, a preferred canine TAg1 nucleic acid molecule encodes a canine TAg1 protein comprising at least about 5 amino acids in length, preferably at least about 10 amino acids in length, preferably at least about 15 amino acids in length, preferably at least about 20 amino acids in length, preferably at least about 25 amino acids in length, preferably at least about 35 amino acids in length, preferably at least about 50 amino acids in length, preferably at least about 100 amino acids in length, preferably at least about 200 amino acids in length, preferably at least about 250 amino acids in length, preferably at least about 300 amino acids in length, and preferably at least about 310 amino acids in length.

In yet another embodiment, a preferred canine TAg1 nucleic acid molecule of the present invention comprises an apparently full-length TAg1 coding region, i.e., the preferred nucleic acid molecule encodes an apparently full-length TAg1 protein with or without an initiating methionine.

Knowing the nucleic acid sequences of certain canine TAg1 nucleic acid molecules of the present invention allows one skilled in the art to, for example, (a) make copies of those nucleic acid molecules, (b) obtain nucleic acid molecules including at least a portion of such nucleic acid molecules (e.g., nucleic acid molecules including full- length genes, full-length coding regions, regulatory control sequences, truncated coding regions), and (c) obtain other canine TAg1 nucleic acid molecules. Such nucleic acid molecules can be obtained in a variety of ways including screening appropriate expression libraries with antibodies of the present invention; traditional cloning techniques using oligonucleotide probes of the present invention to screen appropriate libraries; and PCR amplification of appropriate libraries or DNA using oligonucleotide primers of the present invention. Preferred libraries to screen or from which to amplify nucleic acid molecules include Canis familiaris placental or soft tissue sarcoma cDNA libraries as well as genomic DNA libraries. Similarly, preferred DNA sources from which to amplify nucleic acid molecules include Canis familiaris placental or soft tissue sarcoma cDNA and genomic DNA. Techniques to clone and amplify genes are disclosed, for example, in Sambrook et al., ibid.

The present invention also includes nucleic acid molecules that are oligonucleotides capable of hybridizing, under stringent hybridization conditions, with complementary regions of other, preferably longer, nucleic acid molecules of the present invention such as those comprising Canis familiaris TAg1 nucleic acid molecules or other canine TAg1 nucleic acid molecules. Oligonucleotides of the present invention can be RNA, DNA, or derivatives of either. The minimum size of such oligonucleotides is the size required for formation of a stable hybrid between an oligonucleotide and a complementary sequence on a nucleic acid molecule of the present invention. A preferred oligonucleotide of the present invention has a maximum size of preferably about 200 nucleotides. The present invention includes oligonucleotides that can be used as, for example, probes to identify nucleic acid molecules, primers to produce nucleic acid molecules, or therapeutic reagents to inhibit canine TAg1 protein production or activity (e.g., as antisense-, triplex formation-, ribozyme- and/or RNA drug-based reagents). The present invention also includes the use of such oligonucleotides to protect animals from disease using one or more of such technologies. Appropriate oligonucleotide-containing therapeutic compositions can be administered to an animal using techniques known to those skilled in the art.

One embodiment of the present invention includes a recombinant vector, which includes at least one isolated nucleic acid molecule of the present invention, inserted into any vector capable of delivering the nucleic acid molecule into a host cell. Such a vector contains heterologous nucleic acid sequences, that is nucleic acid sequences that are not naturally found adjacent to nucleic acid molecules of the present invention and that preferably are derived from a species other than the species from which the nucleic acid molecule(s) are derived. The vector can be either RNA or DNA, either prokaryotic or eukaryotic, and typically is a virus or a plasmid. Recombinant vectors can be used in the cloning, sequencing, and/or otherwise manipulating of canine TAg1 nucleic acid molecules of the present invention.

One type of recombinant vector, referred to herein as a recombinant molecule, comprises a nucleic acid molecule of the present invention operatively linked to an expression vector. The phrase operatively linked refers to insertion of a nucleic acid molecule into an expression vector in a manner such that the molecule is able to be expressed when transformed into a host cell. As used herein, an expression vector is a DNA or RNA vector that is capable of transforming a host cell and of effecting expression of a specified nucleic acid molecule. Preferably, the expression vector is also capable of replicating within the host cell. Expression vectors can be either prokaryotic or eukaryotic, and are typically viruses or plasmids. Expression vectors of the present invention include any vectors that function (i.e., direct gene expression) in recombinant cells of the present invention, including in bacterial, fungal, parasite, insect, other animal, and plant cells. Preferred expression vectors of the present invention can direct gene expression in bacterial, yeast, insect and mammalian cells, and more preferably in the cell types disclosed herein.

In particular, expression vectors of the present invention contain regulatory sequences such as transcription control sequences, translation control sequences, origins of replication, and other regulatory sequences that are compatible with the recombinant cell and that control the expression of nucleic acid molecules of the present invention. In particular, recombinant molecules of the present invention include transcription control sequences. Transcription control sequences are sequences which control the initiation, elongation, and termination of transcription. Particularly important transcription control sequences are those which control transcription initiation, such as promoter, enhancer, operator and repressor sequences. Suitable transcription control sequences include any transcription control sequence that can function in at least one of the recombinant cells of the present invention. A variety of such transcription control sequences are known to those skilled in the art. Preferred transcription control sequences include those which function in bacterial, yeast, or insect and mammalian cells, such as, but not limited to, tac, lac, trp, trc, oxy-pro, omp/lpp, rrnB, bacteriophage lambda (such as lambda p_{L} and lambda p_{R} and fusions that include such promoters), bacteriophage T7, T7lac, bacteriophage T3, bacteriophage SP6, bacteriophage SP01, Caulobacter, metallothionein, alpha-mating factor, Pichia alcohol oxidase, alphavirus subgenomic promoter, antibiotic resistance gene, baculovirus, Heliothis zea insect virus, vaccinia virus, herpesvirus, raccoon poxvirus, other poxvirus, adenovirus, cytomegalovirus (such as immediate early promoter), simian virus 40, retrovirus, actin, retroviral long terminal repeat, Rous sarcoma virus, heat shock, phosphate and nitrate transcription control sequences as well as other sequences capable of controlling gene expression in prokaryotic or eukaryotic cells. Suitable transcription control sequences include tissue-specific promoters and enhancers as well as lymphokine-inducible promoters (e.g., promoters inducible by interferons or interleukins).

Suitable and preferred nucleic acid molecules to include in recombinant vectors of the present invention are as disclosed herein. Particularly preferred nucleic acid molecules to include in recombinant vectors, and particularly in recombinant molecules, include ncapTAg1₁₂₃₆, ncapTAg1₉₇₈, ncapTAg1₁₄₄, ncapTAg1₂₂₂, ncapTAg1₆₁₂, ncasTAg1₁₂₆₉, ncasTAg1₉₈₇, ncasTAg1₁₅₃, ncasTAg1₂₂₂, ncasTAg1₆₁₂, rncasTAg1₉₈₇, ncasTAg1₆₈₁, and/or rncasTAg1₆₈₁.

Recombinant molecules of the present invention may also (a) contain secretory signals (i.e., signal segment nucleic acid sequences) to enable an expressed TAg1 protein of the present invention to be secreted from the cell that produces the protein and/or (b) contain fusion sequences which lead to the expression of nucleic acid molecules of the present invention as fusion proteins. Examples of suitable signal segments include any signal segment capable of directing the secretion of a protein of the present invention. Preferred signal segments include, but are not limited to, tissue plasminogen activator (t-PA), interferon, yeast alpha factor, interleukin, growth hormone, histocompatibility and viral envelope glycoprotein signal segments. Suitable fusion segments encoded by fusion segment nucleic acids are disclosed herein. In addition, a nucleic acid molecule of the present invention can be joined to a fusion segment that directs the encoded protein to the proteosome, such as a ubiquitin fusion segment. Eukaryotic recombinant molecules may also include intervening and/or untranslated sequences surrounding and/or within the nucleic acid sequences of nucleic acid molecules of the present invention.

Another embodiment of the present invention includes a recombinant cell comprising a host cell transformed with one or more recombinant molecules of the present invention. Transformation of a nucleic acid molecule into a cell can be accomplished by any method by which a nucleic acid molecule can be inserted into the cell. Transformation techniques include, but are not limited to, transfection, electroporation, microinjection, lipofection, adsorption, and protoplast fusion. A recombinant cell may remain unicellular or may grow into a tissue, organ or a multicellular organism. It is to be noted that a cell line refers to any recombinant cell of the present invention that is not a transgenic animal. Transformed nucleic acid molecules of the present invention can remain extrachromosomal or can integrate into one or more sites within a chromosome of the transformed (i.e., recombinant) cell in such a manner that their ability to be expressed is retained. Preferred nucleic acid molecules with which to transform a cell include TAg1 nucleic acid molecules disclosed herein. Particularly preferred nucleic acid molecules with which to transform a cell include TAg1₁₂₃₆, ncapTAg1₉₇₈, ncapTAg1₁₄₄, ncapTAg1₂₂₂, ncapTAg1₆₁₂, ncasTAg1₁₂₆₉, ncasTAg1₉₈₇, ncasTAg1₁₅₃, ncasTAg1₂₂₂, ncasTAg1₆₁₂, rncasTAg1₉₈₇, ncasTAg1₆₈₁, and/or rncasTAg1₆₈₁.

Suitable host cells to transform include any cell that can be transformed with a nucleic acid molecule of the present invention. Host cells can be either untransformed cells or cells that are already transformed with at least one nucleic acid molecule (e.g., nucleic acid molecules encoding one or more proteins of the present invention and/or other proteins useful in the production of multivalent vaccines). Host cells of the present invention either can be endogenously (i.e., naturally) capable of producing canine TAg1 proteins of the present invention or can be capable of producing such proteins after being transformed with at least one nucleic acid molecule of the present invention. Host cells of the present invention can be any cell capable of producing at least one protein of the present invention, and include bacterial, fungal (including yeast), parasite, insect, animal and plant cells. Preferred host cells include bacterial, mycobacterial, yeast, insect and mammalian cells. More preferred host cells include Salmonella, Escherichia, Bacillus, Caulobacter, Listeria, Pichia, Hansenula, Saccharomyces, Spodoptera, Mycobacteria, Trichoplusia, BHK (baby hamster kidney) cells, MDCK cells (Madin-Darby canine kidney cell line), CRFK cells (Crandell feline kidney cell line), CV-1 cells (African monkey kidney cell line used, for example, to culture raccoon poxvirus), COS (e.g., COS-7) cells, and Vero cells. Particularly preferred host cells are Escherichia coli, including E. coli K-12 derivatives; Salmonella typhi; Salmonella typhimurium, including attenuated strains such as UK-1_{χ}3987 and SR-11_{χ}4072; Pichia pastoris, Spodoptera frugiperda; Trichoplusia ni; BHK cells; MDCK cells; CRFK cells; CV-1 cells; COS cells; Vero cells; and non-tumorigenic mouse myoblast G8 cells (e.g., ATCC CRL 1246). Appropriate mammalian cell hosts include other kidney cell lines, other fibroblast cell lines (e.g., human, murine or chicken embryo fibroblast cell lines), myeloma cell lines, Chinese hamster ovary cells, mouse NIH/3T3 cells, LMTK³¹ cells and/or HeLa cells. In one embodiment, the proteins may be expressed as heterologous proteins in myeloma cell lines employing immunoglobulin promoters.

A recombinant cell is preferably produced by transforming a host cell with one or more recombinant molecules, each comprising one or more nucleic acid molecules of the present invention operatively linked to an expression vector containing one or more transcription control sequences, examples of which are disclosed herein.

A recombinant cell of the present invention includes any cell transformed with at least one of any nucleic acid molecule of the present invention. Suitable and preferred nucleic acid molecules as well as suitable and preferred recombinant molecules with which to transfer cells are disclosed herein.

Recombinant cells of the present invention can also be co-transformed with one or more recombinant molecules including canine TAg1 nucleic acid molecules encoding one or more proteins of the present invention and one or more other nucleic acid molecules encoding other protective compounds, as disclosed herein (e.g., to produce multivalent vaccines).

Recombinant DNA technologies can be used to improve expression of transformed nucleic acid molecules by manipulating, for example, the number of copies of the nucleic acid molecules within a host cell, the efficiency with which those nucleic acid molecules are transcribed, the efficiency with which the resultant transcripts are translated, and the efficiency of post-translational modifications. Recombinant techniques useful for increasing the expression of nucleic acid molecules of the present invention include, but are not limited to, operatively linking nucleic acid molecules to high-copy number plasmids, integration of the nucleic acid molecules into one or more host cell chromosomes, addition of vector stability sequences to plasmids, substitutions or modifications of transcription control signals (e.g., promoters, operators, enhancers), substitutions or modifications of translational control signals (e.g., ribosome binding sites, Shine-Dalgarno sequences), modification of nucleic acid molecules of the present invention to correspond to the codon usage of the host cell, deletion of sequences that destabilize transcripts, and use of control signals that temporally separate recombinant cell growth from recombinant enzyme production during fermentation. The activity of an expressed recombinant protein of the present invention may be improved by fragmenting, modifying, or derivatizing nucleic acid molecules encoding such a protein.

Isolated canine TAg1 proteins of the present invention can be produced in a variety of ways, including production and recovery of natural proteins, production and recovery of recombinant proteins, and chemical synthesis of the proteins. In one embodiment, an isolated protein of the present invention is produced by culturing a cell capable of expressing the protein under conditions effective to produce the protein, and recovering the protein. A preferred cell to culture is a recombinant cell of the present invention. Effective culture conditions include, but are not limited to, effective media, bioreactor, temperature, pH and oxygen conditions that permit protein production. An effective, medium refers to any medium in which a cell is cultured to produce a canine TAg1 protein of the present invention. Such medium typically comprises an aqueous medium having assimilable carbon, nitrogen and phosphate sources, and appropriate salts, minerals, metals and other nutrients, such as vitamins. Cells of the present invention can be cultured in conventional fermentation bioreactors, shake flasks, test tubes, microtiter dishes, and petri plates. Culturing can be carried out at a temperature, pH and oxygen content appropriate for a recombinant cell. Such culturing conditions are within the expertise of one of ordinary skill in the art. Examples of suitable conditions are included in the Examples section.

Depending on the vector and host system used for production, resultant proteins of the present invention may either remain within the recombinant cell; be secreted into the fermentation medium; be secreted into a space between two cellular membranes, such as the periplasmic space in E. coli; or be retained on the outer surface of a cell or viral membrane.

The phrase “recovering the protein”, as well as similar phrases, refers to collecting the whole fermentation medium containing the protein and need not imply additional steps of separation or purification. Proteins of the present invention can be purified using a variety of standard protein purification techniques, such as, but not limited to, affinity chromatography, ion exchange chromatography, filtration, electrophoresis, hydrophobic interaction chromatography, gel filtration chromatography, reverse phase chromatography, concanavalin A chromatography, chromatofocusing and differential solubilization. Proteins of the present invention are preferably retrieved in “substantially pure” form. As used herein, “substantially pure” refers to a purity that allows for the effective use of the protein as a therapeutic composition or diagnostic. A therapeutic composition for animals, for example, should exhibit no substantial toxicity and preferably should be capable of stimulating the production of antibodies in a treated animal.

The present invention also includes isolated (i.e., removed from their natural milieu) antibodies that selectively bind to a canine TAg1 protein of the present invention or a mimetope thereof (e.g., anti-canine TAg1 antibodies). As used herein, the term “selectively binds to” a TAg1 protein refers to the ability of antibodies of the present invention to preferentially bind to specified proteins and mimetopes thereof of the present invention. Binding can be measured using a variety of methods standard in the art including enzyme immunoassays (e.g., ELISA), immunoblot assays, etc.; see, for example, Sambrook et al., ibid., and Harlow, et al., 1988, Antibodies, a Laboratory Manual, Cold Spring Harbor Labs Press; Harlow et al., ibid., is incorporated by reference herein in its entirety. An anti-TAg1 antibody of the present invention preferably selectively binds to a canine TAg1 protein in such a way as to inhibit the function of that protein.

Isolated antibodies of the present invention can include antibodies in serum, or antibodies that have been purified to varying degrees. Antibodies of the present invention can be polyclonal or monoclonal, or can be functional equivalents such as antibody fragments and genetically-engineered antibodies, including single chain antibodies or chimeric antibodies that can bind to one or more epitopes.

A preferred method to produce antibodies of the present invention includes (a) administering to an animal an effective amount of a protein, peptide or mimetope thereof of the present invention to produce the antibodies and (b) recovering the antibodies. In another method, antibodies of the present invention are produced recombinantly using techniques as heretofore disclosed to produce TAg1 proteins of the present invention. Antibodies raised against defined proteins or mimetopes can be advantageous because such antibodies are not substantially contaminated with antibodies against other substances that might otherwise cause interference in a diagnostic assay or side effects if used in a therapeutic composition.

Antibodies of the present invention have a variety of potential uses that are within the scope of the present invention. For example, such antibodies can be used (a) as therapeutic compounds to passively immunize an animal in order to protect the animal from disease susceptible to treatment by such antibodies, (b) as reagents in assays to detect disease and/or (c) as tools to screen expression libraries and/or to recover desired proteins of the present invention from a mixture of proteins and other contaminants. Furthermore, antibodies of the present invention can be used to target cytotoxic agents. Targeting can be accomplished by conjugating (i.e., stably joining) such antibodies to the cytotoxic agents using techniques known to those skilled in the art. Suitable cytotoxic agents are known to those skilled in the art.

The present invention also includes a therapeutic composition comprising at least one canine TAg1-based compound of the present invention in combination with at least one additional compound protective against one or more infectious agents. Examples of such compounds and infectious agents are disclosed herein.

Therapeutic compositions of the present invention can be administered to any animal susceptible to such therapy, preferably to mammals, and more preferably to dogs, cats, humans, ferrets, horses, cattle, sheep and other pets, economic food animals, work animals and/or zoo animals. Preferred animals to protect against disease include dogs, cats, humans and ferrets, with dogs and cats being particularly preferred.

In order to protect an animal from disease, a therapeutic composition of the present invention is administered to the animal in an effective manner such that the composition is capable of protecting that animal from disease. Therapeutic compositions of the present invention can be administered to animals prior to entering a disease state in order to prevent disease (i.e., as a preventative vaccine) and/or can be administered to animals after entering a disease state in order to treat disease (i.e., as a therapeutic vaccine).

Therapeutic compositions of the present invention can be formulated in an excipient that the animal to be treated can tolerate. Examples of such excipients include water, saline, Ringer's solution, dextrose solution, Hank's solution, and other aqueous physiologically balanced salt solutions. Nonaqueous vehicles, such as fixed oils, sesame oil, ethyl oleate, or triglycerides may also be used. Other useful formulations include suspensions containing viscosity enhancing agents, such as sodium carboxymethylcellulose, sorbitol, or dextran. Excipients can also contain minor amounts of additives, such as substances that enhance isotonicity and chemical stability. Examples of buffers include phosphate buffer, bicarbonate buffer and Tris buffer, while examples of preservatives include thimerosal, o-cresol, formalin and benzyl alcohol. Standard formulations can either be liquid injectables or solids which can be taken up in a suitable liquid as a suspension or solution for injection. Thus, in a non-liquid formulation, the excipient can comprise dextrose, human serum albumin, preservatives, etc., to which sterile water or saline can be added prior to administration.

In one embodiment of the present invention, a therapeutic composition can include an adjuvant. Adjuvants are agents that are capable of enhancing the immune response of an animal to a specific antigen. Suitable adjuvants include, but are not limited to, cytokines, chemokines, and compounds that induce the production of cytokines and chemokines (e.g., granulocyte macrophage colony stimulating factor (GM-CSF), granulocyte colony stimulating factor (G-CSF), macrophage colony stimulating factor (M-CSF), colony stimulating factor (CSF), erythropoietin (EPO), interleukin 2 (IL-2), interleukin-3 (IL-3), interleukin 4 (IL-4), interleukin 5 (IL-5), interleukin 6 (IL-6), interleukin 7 (IL-7), interleukin 8 (IL-8), interleukin 10 (IL-10), interleukin 12 (IL-12), interferon gamma, interferon gamma inducing factor I (IGIF), transforming growth factor beta, RANTES (regulated upon activation, normal T-cell expressed and presumably secreted), macrophage inflammatory proteins (e.g., MIP-1 alpha and MIP-1 beta), and Leishmania elongation initiating factor (LEIF)); bacterial components (e.g., endotoxins, in particular superantigens, exotoxins and cell wall components); aluminum-based salts; calcium-based salts; silica; polynucleotides; toxoids; serum proteins, viral coat proteins; block copolymer adjuvants (e.g., Hunter's Titermax™ adjuvant (Vaxcel™, Inc. Norcross, Ga.), Ribi adjuvants (Ribi ImmunoChem Research, Inc., Hamilton, Mont.); and saponins and their derivatives (e.g., Quil A (Superfos Biosector A/S, Denmark). Protein adjuvants of the present invention can be delivered in the form of the protein themselves or of nucleic acid molecules encoding such proteins using the methods described herein.

In one embodiment of the present invention, a therapeutic composition can include a carrier. Carriers include compounds that increase the half-life of a therapeutic composition in the treated animal. Suitable carriers include, but are not limited to, polymeric controlled release vehicles, biodegradable implants, liposomes, bacteria, viruses, other cells, oils, esters, and glycols.

One embodiment of the present invention is a controlled release formulation that is capable of slowly releasing a composition of the present invention into an animal. As used herein, a controlled release formulation comprises a composition of the present invention in a controlled release vehicle. Suitable controlled release vehicles include, but are not limited to, biocompatible polymers, other polymeric matrices, capsules, microcapsules, microparticles, bolus preparations, osmotic pumps, diffusion devices, liposomes, lipospheres, and transdermal delivery systems. Other controlled release formulations of the present invention include liquids that, upon administration to an animal, form a solid or a gel in situ. Preferred controlled release formulations are biodegradable (i.e., bioerodible).

A preferred controlled release formulation of the present invention is capable of releasing a composition of the present invention into the blood of the treated animal at a constant rate sufficient to attain therapeutic dose levels of the composition to protect an animal from disease. The therapeutic composition is preferably released over a period of time ranging from about 1 to about 12 months. A controlled release formulation of the present invention is capable of effecting a treatment preferably for at least about 1 month, more preferably for at least about 3 months, even more preferably for at least about 6 months, even more preferably for at least about 9 months, and even more preferably for at least about 12 months.

Therapeutic compositions of the present invention can be administered to animals prior to entering a disease state in order to prevent disease and/or can be administered to animals after entering a disease state in order to treat disease. For example, proteins, mimetopes thereof, and antibodies thereof can be used as immunotherapeutic agents. Acceptable protocols to administer therapeutic compositions in an effective manner include individual dose size, number of doses, frequency of dose administration, and mode of administration. Determination of such protocols can be accomplished by those skilled in the art. A suitable single dose is a dose that is capable of protecting an animal from disease when administered one or more times over a suitable time period. For example, a preferred single dose of a protein, mimetope or antibody therapeutic composition is from about 1 microgram (μg) to about 10 milligrams (mg) of the therapeutic composition per kilogram body weight of the animal. Booster vaccinations can be administered from about 2 weeks to several years after the original administration. Booster administrations preferably are administered when the immune response of the animal becomes insufficient to protect the animal from disease. A preferred administration schedule is one in which from about 10 μg to about 1 mg of the therapeutic composition per kg body weight of the animal is administered from about one to about two times over a time period of from about 2 weeks to about 12 months. Modes of administration can include, but are not limited to, subcutaneous, intradermal, intravenous, intranasal, oral, transdermal and intramuscular routes.

According to one embodiment, a nucleic acid molecule of the present invention can be administered to an animal in a fashion to enable expression of that nucleic acid molecule into a protective protein or protective RNA (e.g., antisense RNA, ribozyme, triple helix forms or RNA drug) in the animal. Nucleic acid molecules can be delivered to an animal in a variety of methods including, but not limited to, (a) administering a naked (i.e., not packaged in a viral coat or cellular membrane) nucleic acid as a genetic vaccine (e.g., as naked DNA or RNA molecules, such as is taught, for example in Wolff et al., 1990, Science 247, 1465-1468) or (b) administering a nucleic acid molecule packaged as a recombinant virus vaccine or as a recombinant cell vaccine (i.e., the nucleic acid molecule is delivered by a viral or cellular vehicle).

A genetic (i.e., naked nucleic acid) vaccine of the present invention includes a nucleic acid molecule of the present invention and preferably includes a recombinant molecule of the present invention that preferably is replication, or otherwise amplification, competent. A genetic vaccine of the present invention can comprise one or more nucleic acid molecules of the present invention in the form of, for example, a dicistronic recombinant molecule. Preferred genetic vaccines include at least a portion of a viral genome (i.e., a viral vector). Preferred viral vectors include those based on alphaviruses, poxviruses, adenoviruses, herpesviruses, picornaviruses, and retroviruses, with those based on alphaviruses (such as sindbis or Semliki forest virus), species-specific herpesviruses and poxviruses being particularly preferred. Any suitable transcription control sequence can be used, including those disclosed as suitable for protein production. Particularly preferred transcription control sequences include cytomegalovirus immediate early (preferably in conjunction with Intron-A), Rous sarcoma virus long terminal repeat, and tissue-specific transcription control sequences, as well as transcription control sequences endogenous to viral vectors if viral vectors are used. The incorporation of a “strong” polyadenylation signal is also preferred.

Genetic vaccines of the present invention can be administered in a variety of ways, with intramuscular, subcutaneous, intradermal, transdermal, intranasal and oral routes of administration being preferred. A preferred single dose of a genetic vaccine ranges from about 1 nanogram (ng) to about 600 μg, depending on the route of administration and/or method of delivery, as can be determined by those skilled in the art. Suitable delivery methods include, for example, by injection, as drops, aerosolized and/or topically. Genetic vaccines of the present invention can be contained in an aqueous excipient (e.g., phosphate buffered saline) alone or in a carrier (e.g., lipid-based vehicles).

A recombinant virus vaccine of the present invention includes a recombinant molecule of the present invention that is packaged in a viral coat and that can be expressed in an animal after administration. Preferably, the recombinant molecule is packaging- or replication-deficient and/or encodes an attenuated virus. A number of recombinant viruses can be used, including, but not limited to, those based on alphaviruses, poxviruses, adenoviruses, herpesviruses, picornaviruses, and retroviruses. Preferred recombinant virus vaccines are those based on alphaviruses (such as Sindbis virus), raccoon poxviruses, species-specific herpesviruses and species-specific poxviruses. An example of methods to produce and use alphavirus recombinant virus vaccines are disclosed in PCT Publication No. WO 94/17813, by Xiong et al., published Aug. 18, 1994, which is incorporated by reference herein in its entirety.

When administered to an animal, a recombinant virus vaccine of the present invention infects cells within the immunized animal and directs the production of a protective protein or RNA nucleic acid molecule that is capable of protecting the animal from disease as disclosed herein. For example, a recombinant virus vaccine comprising a TAg1 nucleic acid molecule of the present invention is administered according to a protocol that results in the animal producing a sufficient immune response to protect itself from disease. A preferred single dose of a recombinant virus vaccine of the present invention is from about 1×10⁴ to about 1×10⁸ virus plaque forming units (pfu) per kilogram body weight of the animal. Administration protocols are similar to those described herein for protein-based vaccines, with subcutaneous, intramuscular, intranasal and oral administration routes being preferred.

A recombinant cell vaccine of the present invention includes recombinant cells of the present invention that express at least one protein of the present invention. Preferred recombinant cells for this embodiment include Salmonella, E. coli, Listeria, Mycobacterium, S. frugiperda, yeast, (including Saccharomyces cerevisiae and Pichia pastoris), BHK, CV-1, myoblast G8, COS (e.g., COS-7), Vero, MDCK and CRFK recombinant cells. Recombinant cell vaccines of the present invention can be administered in a variety of ways but have the advantage that they can be administered orally, preferably at doses ranging from about 10⁸ to about 10¹² cells per kilogram body weight. Administration protocols are similar to those described herein for protein-based vaccines. Recombinant cell vaccines can comprise whole cells, cells stripped of cell walls or cell lysates.

The efficacy of a therapeutic composition of the present invention to protect an animal from disease can be tested in a variety of ways including, but not limited to, detection of protective antibodies (using, for example, proteins or mimetopes of the present invention), detection of cellular immunity within the treated animal, or challenge of the treated animal with disease causative agent to determine whether the treated animal is resistant to disease. In one embodiment, therapeutic compositions can be tested in animal models such as mice. Such techniques are known to those skilled in the art.

One preferred embodiment of the present invention is the use of canine TAg1 proteins, nucleic acid molecules, antibodies and inhibitors of the present invention, to protect an animal from disease. For example, an isolated protein or mimetope thereof is administered in an amount and manner that elicits (i.e., stimulates) an immune response that is sufficient, upon interaction with a native TAg1 protein, to protect the animal from the disease. Similarly, an antibody of the present invention, when administered to an animal in an effective manner, is administered in an amount so as to be present in the animal at a titer that is sufficient, upon interaction of that antibody with a native TAg1 protein, to protect the animal from the disease, at least temporarily. Oligonucleotide nucleic acid molecules of the present invention can also be administered in an effective manner, thereby reducing expression of canine TAg1 proteins in order to interfere with development of a disease state targeted in accordance with the present invention. Particularly preferred therapeutic compositions include canine TAg1-based therapeutic compositions of the present invention. Such compositions include canine TAg1 nucleic acid molecules, canine TAg1 proteins and mimetopes thereof, anti-canine TAg1 antibodies, and inhibitors of canine TAg1 protein function. Therapeutic compositions are administered to animals in a manner effective to protect the animals from disease. Additional protection may be obtained by administering additional protective compounds, as disclosed herein.

One therapeutic composition of the present invention includes an inhibitor of canine TAg1 protein function, i.e., a compound capable of substantially interfering with the function of a canine TAg1 protein susceptible to inhibition. An inhibitor of TAg1 protein function can be identified using canine TAg1 proteins of the present invention. A preferred method to identify a compound capable of inhibiting canine TAg1 protein activity includes contacting an isolated canine TAg1 protein having an amino acid sequence selected from the group consisting of SEQ ID NO:3, SEQ ID NO:9, SEQ ID NO:12, SEQ ID NO:15, SEQ ID NO:18, SEQ ID NO:24, SEQ ID NO:27, SEQ ID NO:30, SEQ ID NO:33, SEQ ID NO:36, SEQ ID NO:39, SEQ ID NO:42, SEQ ID NO:45, SEQ ID NO:48, SEQ ID NO:51, SEQ ID NO:54, SEQ ID NO:57, SEQ ID NO:60, SEQ ID NO:63, SEQ ID NO:66, SEQ ID NO:69, and/or SEQ ID NO:74 with a putative inhibitory compound under conditions in which, in the absence of said compound, said protein has TAg1 activity; and determining if said putative inhibitory compound inhibits said activity.

It is also within the scope of the present invention to use isolated proteins, mimetopes, nucleic acid molecules and antibodies of the present invention as diagnostic reagents to detect disease. One embodiment of the present invention is a method to detect disease which includes the steps of: (a) contacting an isolated anti-TAg1 antibody with a putative TAg1 protein-containing composition under conditions suitable for formation of a anti-TAg1:TAg1 protein complex; and (b) detecting the presence of TAg1 protein by detecting the anti-TAg1:TAg1 protein complex. Presence of such a anti-TAg1:TAg1 protein complex indicates that the animal is producing TAg1. Preferred TAg1 protein to detect using an anti-TAg1 antibody include any mammalian TAg1, with human TAg1, canine TAg1, feline TAg1, equine TAg1, murine TAg1 and rat TAg1 being more preferred, with human TAg1, canine TAg1, feline TAg1 and equine TAg1 being particularly preferred.

Another embodiment of the present invention is a method to detect disease which includes the steps of: (a) contacting an isolated TAg1 protein with a putative anti-TAg1 antibody-containing composition under conditions suitable for formation of a anti-TAg1:TAg1 protein complex; and (b) detecting the presence of anti-TAg1 antibody by detecting the anti-TAg1:TAg1 protein complex. Presence of such a anti-TAg1:TAg1 protein complex indicates that the animal is producing anti-TAg1 antibody in response to the presence of TAg1. Preferred anti-Tag1 antibody to detect using a TAg1 protein include any mammalian anti-TAg1, with human anti-TAg1, canine anti-TAg1, feline anti-TAg1, equine anti-TAg1, murine anti-TAg1 and rat anti-TAg1 being more preferred, with human anti-TAg1, canine anti-TAg1, feline anti-TAg1 and equine anti-TAg1 being particularly preferred.

Yet another embodiment of the present invention is a method to detect disease which utilizes TAg1 nucleic acid molecules of the present invention, including for example PCR, hybridization and other techniques known to those of skill in the art.

Animals in which to detect TAg1 protein include mammals and birds, with humans, dogs, cats, horses and other pets, work and/or economic food animals being preferred. Particularly preferred animals in which to detect TAg1 protein are humans, dogs, cats and horses. As used herein, canine refers to any member of the dog family, including domestic dogs, wild dogs and zoo dogs. Examples of dogs include, but are not limited to, domestic dogs, wild dogs, foxes, wolves, jackals and coyotes. As used herein, feline refers to any member of the cat family, including domestic cats, wild cats and zoo cats. Examples of cats include, but are not limited to, domestic cats, wild cats, lions, tigers, leopards, panthers, cougars, bobcats, lynx, jaguars, cheetahs, and servals. As used herein, equine refers to any member of the horse family, including, but are not limited to, domestic horses, wild horses and zoo horses.

As used herein, the term “contacting” refers to combining or mixing, in this case a putative TAg1-containing composition with an anti-TAg1 antibody. Formation of a complex between an anti-TAg1 antibody and a TAg1 protein refers to the ability of the anti-TAg1 antibody molecule to selectively bind to the TAg1 protein in order to form a stable complex that can be measured (i.e., detected). As used herein, the term “selectively binds” to a TAg1 protein refers to the ability of an anti-TAg1 antibody of the present invention to preferentially bind to TAg1, without being able to substantially bind to non-TAg1 antigens. Binding between an anti-TAg1 antibody and a TAg1 protein is effected under conditions suitable to form a complex; such conditions (e.g., appropriate concentrations, buffers, temperatures, reaction times) as well as methods to optimize such conditions are known to those skilled in the art, and examples are disclosed herein. Examples of complex formation conditions are also disclosed in, for example, Sambrook et al., ibid.

The term “contacting” may also refer to combining or mixing, a putative anti-TAg1 antibody containing composition with a TAg1 antibody. Formation of a complex between a TAg1 protein and an anti-TAg1 antibody refers to the ability of the TAg1 protein and anti-TAg1 antibody molecule to selectively bind in order to form a stable complex that can be measured (i.e., detected). As used herein, the term “selectively binds” to an anti-TAg1 antibody of the present invention refers to the ability of a TAg1 protein to preferentially bind to anti-TAg1 antibody, without being able to substantially bind to non-anti-TAg1 antibodies. Binding between a TAg1 protein and an anti-TAg1 antibody is effected under conditions suitable to form a complex; such conditions (e.g., appropriate concentrations, buffers, temperatures, reaction times) as well as methods to optimize such conditions are known to those skilled in the art, and examples are disclosed herein. Examples of complex formation conditions are also disclosed in, for example, Sambrook et al., ibid.

As used herein, the term “detecting complex formation” refers to determining if any complex is formed, i.e., assaying for the presence (i.e., existence) of a complex. If complexes are formed, the amount of complexes formed can, but need not be, determined. Complex formation, or selective binding, between an anti-TAg1 antibody and any TAg1 protein in the composition can be measured (i.e., detected, determined) using a variety of methods standard in the art (see, for example, Sambrook et al. ibid.), examples of which are disclosed herein.

In one embodiment, a putative TAg1-containing composition of the present method includes a biological sample from an animal. A suitable biological sample includes, but is not limited to, a bodily fluid composition or a cellular composition. A bodily fluid refers to any fluid that can be collected (i.e., obtained) from an animal, examples of which include, but are not limited to, blood, serum, plasma, urine, tears, aqueous humor, cerebrospinal fluid (CSF), saliva, lymph, nasal secretions, tracheobronchial aspirates, milk, feces and fluids obtained through bronchial alveolar lavage. Such a composition of the present method can, but need not be, pretreated to remove at least some other proteins, or other components, present in the fluid that might interfere with detection. A preferred composition of the present method is serum.

In one embodiment a complex can be formed and detected in solution. In another embodiment, a complex can be formed in which one or more members of the complex are immobilized on (e.g., coated onto) a support substrate. Immobilization techniques are known to those skilled in the art. Suitable support substrate materials include, but are not limited to, plastic, glass, gel, celluloid, paper, PVDF (poly-vinylidene-fluoride), nylon, nitrocellulose, and particulate materials such as latex, polystyrene, nylon, nitrocellulose, agarose and magnetic resin. Suitable shapes for support substrate material include, but are not limited to, a well (e.g., microtiter dish well), a plate, a dipstick, a bead, a lateral flow apparatus, a membrane, a filter, a tube, a dish, a celluloid-type matrix, a magnetic particle, and other particulates. A particularly preferred support substrate comprises an ELISA plate, a dipstick, a radioimmunoassay plate, agarose beads, plastic beads, latex beads, immunoblot membranes and immunoblot papers. In one embodiment, a support substrate, such as a particulate, can include a detectable marker.

A preferred method to detect TAg1 protein is an immunosorbent assay. An immunoabsorbent assay of the present invention comprises a capture molecule and an indicator molecule. A capture molecule of the present invention binds to a TAg1 protein in such a manner that the TAg1 protein is immobilized to a support substrate. As such, a capture molecule is preferably immobilized to a support substrate of the present invention prior to exposure of the capture molecule to a putative TAg1-containing composition. An indicator molecule of the present invention detects the presence of a TAg1 protein bound to a capture molecule. As such, an indicator molecule preferably is not immobilized to the same support substrate as a capture molecule prior to exposure of the capture molecule to a putative TAg1-containing composition.

In one embodiment, an anti-TAg1 antibody is used as a capture molecule by being immobilized on a support substrate, such as a microtiter dish well or a dipstick. A biological sample collected from an animal is applied to the support substrate and incubated under conditions suitable (i.e., sufficient) to allow for anti-TAg1:TAg1 protein complex formation bound to the support substrate (i.e., TAg1 protein in a sample binds to an antibody immobilized on a support substrate). Excess non-bound material (i.e., material from the biological sample that has not bound to the antigen), if any, is removed from the support substrate under conditions that retain antibody:TAg1 protein complex binding to the support substrate. An indicator molecule that can selectively bind to a TAg1 protein bound to the antigen is added to the substrate and incubated to allow formation of a complex between the indicator molecule and the anti-TAg1:TAg1 protein complex. Excess indicator molecule is removed, a developing agent is added if required, and the substrate is submitted to a detection device for analysis.

In one embodiment, an immunosorbent assay of the present invention does not utilize a capture molecule. In this embodiment, a biological sample collected from an animal is applied to a support substrate, such as a microtiter dish well or a dipstick, and incubated under conditions suitable to allow for TAg1 protein binding to the support substrate. Any TAg1 protein present in the bodily fluid is immobilized on the support substrate. Excess non-bound material, if any, is removed from the support substrate under conditions that retain TAg1 protein binding to the support substrate. An anti-TAg1 antibody is added to the substrate and incubated to allow formation of a complex between the anti-TAg1 antibody and the TAg1. Preferably, the anti-TAg1 antibody is conjugated to a detectable marker (preferably to biotin, an enzyme label or a fluorescent label). Excess anti-TAg1 antibody is removed, a developing agent is added if required, and the substrate is submitted to a detection device for analysis.

Another preferred method to detect TAg1 protein is a lateral flow assay, examples of which are disclosed in U.S. Pat. No. 5,424,193, issued Jun. 13, 1995, by Pronovost et al.; U.S. Pat. No. 5,415,994, issued May 16, 1995, by Imrich et al; WO 94/29696, published Dec. 22, 1994, by Miller et al.; and WO 94/01775, published Jan. 20, 1994, by Pawlak et al.; each of these patent publications is incorporated by reference herein in its entirety. In one embodiment, a biological sample is placed in a lateral flow apparatus that includes the following components: (a) a support structure defining a flow path; and (b) a capture reagent comprising an anti-TAg1 antibody, the capture reagent being impregnated within the support structure in a labeling zone. Preferably a labeling reagent is added to the lateral flow apparatus and a detecting means is used to detect bound complex. The support structure comprises a material that does not impede the flow of TAg1 protein contained in a biological sample to the capture reagent. Suitable materials for use as a support structure include ionic (i.e., anionic or cationic) material. Examples of such a material include, but are not limited to, nitrocellulose (NC), PVDF, carboxymethylcellulose (CM). The support structure defines a flow path that is lateral and includes a capture zone. The apparatus can further comprise a sample receiving zone located along the flow path, more preferably upstream of the capture reagent. The flow path in the support structure is created by contacting a portion of the support structure downstream of the capture zone, preferably at the end of the flow path, to an absorbent capable of absorbing excess liquid.

Another preferred method to detect TAg1 protein is a lateral flow assay, examples of which are disclosed in U.S. Pat. No. 4,727,019 to Valkirs et al., issued Feb. 23, 1988 which is incorporated by reference herein in its entirety. In one embodiment, a biological sample is placed in a flow through apparatus that includes the following components: (a) a support structure defining a flow path; and (b) a capture reagent comprising an anti-TAg1 antibody, the capture reagent being impregnated within the support structure in a labeling zone. Preferably a labeling reagent is added to the lateral flow apparatus and a detecting means is used to detect bound complex.

The present invention also includes kits to detect TAg1 protein based on each of the disclosed detection methods. Suitable and preferred TAg1 molecules to detect using such a kit are disclosed herein. A preferred kit of the present invention includes those in which the anti-TAg1 is immobilized on a support substrate using a method as described herein. In another embodiment, a preferred kit comprises a lateral flow apparatus, a flow through apparatus or an ELISA. A method and kit of the present invention are useful for diagnosing abnormal conditions in animals that are associated with TAg1. Preferred animals include those disclosed herein, with humans, dogs, cats and horses being more preferred.

One embodiment of the present invention is a method to identify a compound capable of inhibiting TAg1 protein activity. Such a method includes the steps of (a) contacting (e.g., combining, mixing) an isolated TAg1 protein of the present invention, with a putative inhibitory compound under conditions in which, in the absence of the compound, the protein has TAg1 protein activity, and (b) determining if the putative inhibitory compound inhibits the activity. TAg1 protein activity can be determined in a variety of ways known in the art. Such conditions under which a TAg1 protein has TAg1 protein activity include conditions in which a TAg1 protein has a correct three-dimensionally folded structure under physiologic conditions, i.e. physiologic pH, physiologic ionic concentrations, and physiologic temperatures.

Putative inhibitory compounds to screen include antibodies (including fragments and mimetopes thereof), putative substrate analogs, and other, preferably small, organic or inorganic molecules. Methods to determine binding of a putative inhibitory compound to a TAg1 protein of the present invention are known to those of skill in the art and include, for example, determining changes in molecular mass using surface plasmon resonance (e.g., determining light scatter by an inhibitor or a TAg1 protein, before and after contacting the inhibitor or protein with a TAg1 protein or inhibitor, respectively).

A preferred method to identify a compound capable of inhibiting TAg1 protein activity includes contacting an isolated TAg1 protein having an amino acid sequence selected from the group consisting of: (a) a protein comprising an amino acid sequence selected from the group consisting of SEQ ID NO:3, SEQ ID NO:9, SEQ ID NO:12, SEQ ID NO:15, SEQ ID NO:18, SEQ ID NO:24, SEQ ID NO:27, SEQ ID NO:30, SEQ ID NO:33, SEQ ID NO:36, SEQ ID NO:39, SEQ ID NO:42, SEQ ID NO:45, SEQ ID NO:48, SEQ ID NO:51, SEQ ID NO:54, SEQ ID NO:57, SEQ ID NO:60, SEQ ID NO:63, SEQ ID NO:66, SEQ ID NO:69, and SEQ ID NO:74; (b) a protein comprising an at least 6 consecutive amino acid portion identical in sequence to an at least 6 consecutive amino acid portion of a sequence selected from the group consisting of SEQ ID NO:9, SEQ ID NO:24, SEQ ID NO:33, and SEQ ID NO:36; (c) a protein comprising an at least 15 consecutive amino acid portion identical in sequence to an at least 15 consecutive amino acid portion of a sequence selected from the group consisting of SEQ ID NO:12, SEQ ID NO:27, SEQ ID NO:39, and SEQ ID NO:42 and (d) a protein comprising an at least 42 consecutive amino acid portion identical in sequence to an at least 42 consecutive amino acid portion of a sequence selected from the group consisting of SEQ ID NO:3, SEQ ID NO:15, SEQ ID NO:18, SEQ ID NO:30, SEQ ID NO:45, SEQ ID NO:48, SEQ ID NO:51, SEQ ID NO:54, SEQ ID NO:57, SEQ ID NO:60, SEQ ID NO:63, SEQ ID NO:66, SEQ ID NO:69, and SEQ ID NO:74; with a putative inhibitory compound under conditions in which, in the absence of the compound, the TAg1 protein has TAg1 protein activity; and determining if the putative inhibitory compound inhibits the activity.

Another embodiment of the present invention is an assay kit to identify an inhibitor of TAg1 protein activity. This kit comprises an isolated TAg1 protein of the present invention, and a means for determining inhibition of TAg1 protein activity, where the means enables detection of inhibition. Detection of inhibition of TAg1 protein activity identifies a putative inhibitor to be an inhibitor of TAg1 . Means and methods are described herein and are known to those skilled in the art.

The following examples are provided for the purposes of illustration and are not intended to limit the scope of the present invention.

### EXAMPLES

### Example 1

This example describes the isolation, from a canine placental cDNA library, of a nucleic acid molecule encoding TAg1. This example further describes isolation of a TAg1 nucleic acid molecule variant from a canine soft tissue sarcoma cDNA library.

Canine placenta and canine soft tissue sarcoma cDNA libraries were individually constructed as follows. Total RNA was separately extracted from a dog placenta, and from tissue isolated from a canine soft tissue sarcoma, using an acid-guanidinium-phenol-chloroform method similar to that described by Chomzynski, et al, 1987, Anal. Biochem. 162, 156-159. Poly A⁺ selected RNA was separated from each total RNA population by oligo-dT cellulose chromatography using an mRNA Purification Kit, available from Pharmacia Biotech, Newark, N.J., according to the method recommended by the manufacturer. Canine placental and canine soft tissue sarcoma cDNA libraries were each constructed in the lambda-Uni-ZAP™ XR vector, available from Stratagene, La Jolla, Calif., using a Stratagene ZAP-cDNA Synthesis Kit and protocol. The placental and soft tissue sarcoma cDNA libraries were each produced using approximately 5 μg of poly A⁺ RNA.

A. A canine placental TAg1 nucleic acid molecule of about 1236 nucleotides, denoted ncapTAg1₁₂₃₆, was isolated as follows. Using a modification of the protocol described in the cDNA Synthesis Kit, the placental library was screened, using duplicate plaque lifts, with a ³²P-labeled probe encoding a mouse cDNA obtained as an expressed sequence tag (EST), GenBank accession #AA968148, herein represented as SEQ ID NO:1. A positive clone was plaque purified and converted into a double-stranded recombinant molecule using the ExAssist™ helper phage and SOLR™ E. coli according to the in vivo excision protocol described in the ZAP-cDNA Synthesis Kit. Double-stranded plasmid DNA was prepared using the Quantum Prep Plasmid Miniprep Kit, available from Bio-Rad, Hercules, Calif., according to the manufacturers protocol. Plasmid DNA containing nTAg1₁₂₃₆ was sequenced by the Sanger dideoxy chain termination method, using the PRISM™ Ready Dye Terminator Cycle Sequencing Kit with AmpliTaq DNA Polymerase, FS, available from Perkin Elmer Corporation, Norwalk, Conn. PCR extensions were done in the GeneAmp™ Gel Filtration Cartridge, available from Advanced Genetic Technologies, Gaithersburg, Md., following their standard protocol. Samples were resuspended according to ABI protocols and were run on a Perkin-Elmer ABI-PRISM™ 377 Automated DNA Sequencer. DNA sequence analysis, including the compilation of sequences and the determination of open reading frames, was performed using the MacVector™ program, available from IBI, New Haven, Conn. Protein sequence analysis, including the determination of molecular weight and isoelectric point (pI) was performed using the SeqLab Wisconsin Package Version 10.0-UNIX software.

The resulting nucleic acid sequence of ncapTAg1₁₂₃₆ has a coding strand presented herein as SEQ ID NO:2 and a complementary strand presented herein as SEQ ID NO:4. Translation of SEQ ID NO:2 indicates that the sequence contains a full length coding region encoding a protein of about 326 amino acids, denoted herein as PcapTAg1₃₂₆, the amino acid sequence of which is presented in SEQ ID NO:3, assuming an initiation codon extending from nucleotide 34 to nucleotide 36 of SEQ ID NO:2 and a stop codon extending from nucleotide 1012 to nucleotide 1014 of SEQ ID NO:2. The coding region encoding PcapTAg1₃₂₆, referred to herein as ncapTAg1₉₇₈, has a coding strand presented herein as SEQ ID NO:5 and a complementary strand presented herein as SEQ ID NO:7. Analysis of the amino acid sequence of PcapTAg1₃₂₆ (i.e. SEQ ID NO:3) predicts that the protein has an estimated molecular weight of about 36.7 kD and an estimated pI of about 9.53.

Homology searches of the non-redundant protein and nucleic acid sequence databases were performed through the National Center for Biotechnology Information using the BLAST network. The protein database includes SwissProt+PIR+SPUpdate+Genpept+GPUpdate. The nucleotide database includes GenBank+EMBL+DDJB+PDB. A protein homology search performed using SEQ ID NO:3 revealed that amino acids 1 to 48 of SEQ ID NO:3, herein after referred to as SEQ ID NO:9, share less than 20% identity with amino acids 1 to 328 of a Rattus norvegicus p65 protein encoded by accession number AA002424, amino acids 49 to 122 of SEQ ID NO:3, herein after referred to as SEQ ID NO:12, share about 47% identity with amino acids 329 to 397 of the Rattus norvegicus p65 protein encoded by accession number AA002424, and amino acids 123 to 326 of SEQ ID NO:3, herein after referred to as SEQ ID NO:15, share about 77% identity with amino acids 398 to 613 of the Rattus norvegicus p65 protein encoded by accession number AA002424. A nucleotide homology search performed using SEQ ID NO:2, revealed that nucleotides 1 to 144 of SEQ ID NO:2, hereinafter referred to as SEQ ID NO:8, share less than 20% identity with nucleotides 1 to 964 of accession number AA002424, nucleotides 145 to 366 of SEQ ID NO:2, hereinafter referred to as SEQ ID NO:11, share less than 20% identity with nucleotides 965 to 1191 of accession number AA002424, and nucleotides 367 to 978 of SEQ ID NO:2, hereinafter referred to as SEQ ID NO:14, share about 89% identity with nucleotides 1192 to 1639 of accession number AA002424. The homology search described above yielded the surprising result of having a 3′ end with a sequence similar to a nucleic acid molecule encoding a Rattus norvegicus p65 protein but with a very dissimilar 5′ nucleic acid sequence compared to the Rattus norvegicus p65 nucleic acid molecule. Applicants were unable to use 5′ sequence information from the Rattus norvegicus p65 nucleic acid molecule to create primers or otherwise isolate TAg1 nucleic acid molecules of the present invention.

The homology search described above also revealed the existence of a human genomic clone, Accession # HSDJ447F3, which contains sequences sharing a significant level of identity with capTAg1. Percent identity as used herein is determined using the Needleman-Wunsch algorithm available in the DNAsis™ sequence analysis software version 2.0, using the GCG default parameter function.

Furthermore, based upon the homology searches and sequence analysis described above, capTAg1 is predicted to be composed of approximately thirteen exons as follows:

| Exon | | Complement | | Amino |
|---|---|---|---|---|
| No. | Codine SEQ ID NO. | SEQ ID NO. | Nucleotides | Acids |
| 1 | SEQ ID NO:32 | SEQ ID NO:34 | 1-96 | 1-33 |
| 2 | SEQ ID NO:35 | SEQ ID NO:37 | 97-165 | 34-55 |
| 3 | SEQ ID NO:38 | SEQ ID NO:40 | 166-264 | 56-88 |
| 4 | SEQ ID NO:41 | SEQ ID NO:43 | 265-363 | 89-121 |
| 5 | SEQ ID NO:44 | SEQ ID NO:46 | 364-432 | 122-144 |
| 6 | SEQ ID NO:47 | SEQ ID NO:49 | 433-489 | 145-163 |
| 7 | SEQ ID NO:50 | SEQ ID NO:52 | 490-549 | 164-183 |
| 8 | SEQ ID NO:53 | SEQ ID NO:55 | 550-594 | 184-198 |
| 9 | SEQ ID NO:56 | SEQ ID NO:58 | 595-654 | 199-218 |
| 10 | SEQ ID NO:59 | SEQ ID NO:61 | 655-714 | 219-238 |
| 11 | SEQ ID NO:62 | SEQ ID NO:64 | 715-786 | 239-262 |
| 12 | SEQ ID NO:65 | SEQ ID NO:67 | 787-840 | 263-280 |
| 13 | SEQ ID NO:68 | SEQ ID NO:70 | 841-978 | 281-326 |

B. A canine soft tissue sarcoma TAg1 nucleic acid molecule of about 1269 nucleotides, denoted ncasTAg1₁₂₆₉, was isolated as follows. Using a modification of the protocol described in the cDNA Synthesis Kit, capTAg1₁₂₃₆ was ³²P-labeled and used to probe the soft tissue sarcoma cDNA library using duplicate plaque lifts. A plaque purified clone was converted into a double-stranded recombinant molecule using the ExAssist™ helper phage and SOLR™ E. coli according to the in vivo excision protocol described in the ZAP-cDNA Synthesis Kit. Double-stranded plasmid DNA was prepared using the Quantum Prep Plasmid Miniprep Kit, according to the manufacturers protocol. The plasmid, containing ncasTAg1₁₂₆₉ was sequenced and protein sequence analysis, including the determination of molecular weight and isoelectric point(pI) was performed as described above.

The resulting nucleic acid sequence of ncasTAg1₁₂₆₉ has a coding strand presented herein as SEQ ID NO:17 and a complementary strand presented herein as SEQ ID NO:19. Translation of SEQ ID NO:17 indicates that the sequence contains a full length coding region encoding a protein of about 329 amino acids, denoted herein as PcasTAg1₃₂₉, the amino acid sequence of which is presented in SEQ ID NO:18, assuming an initiation codon extending from nucleotide 58 to nucleotide 60 of SEQ ID NO:17 and a stop codon extending from nucleotide 1045 to nucleotide 1047 of SEQ ID NO:17. The coding region encoding PcapTAg1₃₂₉, referred to herein as ncapTAg1₉₈₇, has a coding strand presented herein as SEQ ID NO:20 and a complementary strand presented herein as SEQ ID NO:22. Analysis of the amino acid sequence of PcasTAg1 (i.e. SEQ ID NO:18) predicts that the protein has an estimated molecular weight of about 37 kD and an estimated pI of about 9.53.

A homology search using the BLAST network was performed as described above. A protein homology search performed using SEQ ID NO:18 revealed that amino acids 1 to 51 of SEQ ID NO:18, herein after referred to as SEQ ID NO:24, share less than 20% identity with amino acids 1 to 328 of a Rattus norvegicus p65 protein encoded by accession number AA002424, amino acids 52 to 125 of SEQ ID NO:18, herein after referred to as SEQ ID NO:27, share about 47% identity with amino acids 329 to 397 of a Rattus norvegicus p65 protein encoded by accession number AA002424, and amino acids 126 to 329 of SEQ ID NO:18, herein after referred to as SEQ ID NO:30, share about 71% identity with amino acids 398 to 613 of a Rattus norvegicus p65 protein encoded by accession number AA002424. A nucleotide homology search performed using SEQ ID NO:17, revealed that nucleotides 1 to 153 of SEQ ID NO:17, hereinafter referred to as SEQ ID NO:23, share less than 20% identity with nucleotides 1 to 984 of accession number AA002424, nucleotides 154 to 375 of SEQ ID NO:17, hereinafter referred to as SEQ ID NO:26, share less than 20% identity with nucleotides 965 to 1191 of accession number AA002424, and nucleotides 376 to 987 of SEQ ID NO:17, hereinafter referred to as SEQ ID NO:29, share about 69% identity with nucleotides 1192 to 1835 of accession number AA002424. Percent identity as used herein is determined using the Needleman-Wunsch algorithm available in the DNAsis version 2.0 software, using the GCG default parameter function. PcasTag1₃₂₉ (i.e. SEQ ID NO:18) was further compared to PcapTAg1₃₂₆ (i.e. SEQ ID NO:3) and found to be identical except that PcasTag1₃₂₉ has a 3 amino acid insertion at amino acid 51 which is not found in PcapTAg1₃₂₆.

Based upon the homology searches and sequence analysis described above, casTAg1 is predicted to be composed of approximately thirteen exons as follows:

| Exon No. | Nucleotide Location | Amino Acid Location |
|---|---|---|
| 1 | 1-96 | 1-33 |
| 2 | 97-174 | 34-58 |
| 3 | 175-273 | 59-91 |
| 4 | 274-372 | 92-124 |
| 5 | 373-441 | 125-147 |
| 6 | 442-498 | 148-166 |
| 7 | 449-558 | 167-186 |
| 8 | 559-603 | 187-201 |
| 9 | 604-663 | 202-221 |
| 10 | 664-723 | 222-241 |
| 11 | 724-795 | 242-265 |
| 12 | 796-849 | 266-283 |
| 13 | 850-987 | 284-329 |

### Example 2

This example describes the production of full-length and partial length canine TAg1 proteins in E. coli.

A. A full-length canine TAg1 protein was produced as follows. Nucleic acid molecule ncasTAg1₁₂₆₉ produced as described in Example 1B was used as the template to create rncasTAg1₉₈₇, having a forward strand denoted SEQ ID NO:71 and a reverse strand denoted SEQ ID NO:72, by PCR amplification as described below. The PCR amplification described below incorporates E. coli preferred codon changes into nucleic acid molecule rncasTAg1₉₈₇, but rncasTAg1₉₈₇ still encodes a 329 amino acid protein identical to the protein coded by ncasTAg1₉₈₇, (i.e. a protein identical to ncasTAg1₉₈₇ with amino acid sequence SEQ ID NO:18). The following nucleotide changes were incorporated into rncasTAg1₉₈₇: a change from GGG (encoding GLY) to GGT (encoding GLY) at nucleotides spanning 4 to 6 of SEQ ID NO:71; a change from AGA (encoding ARG) to CGG (encoding ARG) at positions 31 to 33 of SEQ ID NO:71; a change from GGT (encoding GLY) to GGA (encoding GLY) at positions 34 to 36 of SEQ ID NO:71; a change from CCC (encoding PRO) to CCA (encoding PRO) at positions 37 to 39 of SEQ ID NO:71; a change from CGA (encoding ARG) to CGT (encoding ARG) at positions 145 to 147 of SEQ ID NO:71, and a change from AGG (encoding ARG) to CGT (encoding ARG) at positions 148 to 150 of SEQ ID NO:71.

Three PCR amplifications were performed to incorporate the codon changes described above as follows. Sense primer EB 140, having nucleic acid sequence 5′ GTT AAA CAT ATG GGT GCT ACT GGT GAC GCT GAG CAG CCA AGA GGT CCA GGC GGG 3′, denoted herein as SEQ ID NO:78, and antisense primer EB 141, having nucleic acid sequence 5′ GCG GCG GCC ACG ACG TTG AAC TTG CCT GTG CTT TAT CAT 3′, denoted herein as SEQ ID NO:79, were used in a first PCR reaction using ncasTAg1₉₈₇ as template under the following conditions. PCR was performed using a reaction mixture containing 20 nanograms (ng) DNA template, 0.2 millimolar (mM) of each of the dNTP's, 2.5 U Pyrococcus furiosus (Pfu) polymerase, available from Stratagene, La Jolla, Calif., 1 unit (U) Pfu polymerase buffer, 0.5 micromolar (μM) each of primers EB 140 and EB 141 and the reaction was carried out under the following amplification cycles: (1) 1 cycle of 96° C. for 3 minutes; (2) 30 cycles of 96° C. for 1 minute, 55° C. for 1 minute, and 72° C. for 1.5 minutes; and (3) 1 cycle of 72° C. for 7 minutes, hereinafter referred to as “standard PCR conditions”. This reaction resulted in an approximately 160-base pair TAg1 fragment.

A second PCR reaction was performed under standard PCR conditions using sense primer EB142, having a nucleic acid sequence 5′ ATG ATA AAG CAC AGG CAA GTT CAA CGT CGT GGC CGC CGC 3′, denoted herein as SEQ ID NO:80, and antisense primer EB 143, having a nucleic acid sequence 5′ ATG CAC GAA TTC TCA GGT CTG TGG AGG CGC 3′, denoted herein as SEQ ID NO:81, and ncasTAg1₉₈₇ as the template. This reaction resulted in an approximately 880-base pair fragment.

The 160-base pair and 880-base pair PCR products were gel purified and annealed together to form a 987-base pair nucleic acid molecule. This 987-base pair molecule was used as the DNA template in a PCR reaction using sense primer EB 144, having a nucleic acid sequence 5′ GTT AAA CAT ATG GGT GCT ACT GGT GAC GCT GAG 3′; denoted herein as SEQ ID NO:82, and reverse primer EB 145, having nucleic acid sequence 5′ ATG CAC GAA TTC TCA GGT CTG TGG AGG 3′, denoted herein as SEQ ID NO:83. This PCR reaction used the reaction mixture as described in the standard PCR conditions set forth above, but used the following amplification cycles: (1) 1 cycle of 96° C. for 3 minutes; (2) 30 cycles of 96° C. for 1 minute, 55° C. for 1 minute, and 72° C. for 2 minutes; and (3) 1 cycle of 72° C. for 7 minutes. To facilitate cloning, an NdeI site (shown in bold in SEQ ID NO:82) was added to the sense primer and an EcoRI site (shown in bold in SEQ ID NO:83) was added to the antisense primer. The resulting PCR fragment was digested with restriction endonucleases NdeI and EcoRI to produce rncasTAg1₉₈₇, gel purified and ligated into λcro plasmid vector, the production of which is described in U.S. Pat. No. 5,569,603 by Tripp et al., issued Oct. 29, 1996, that had been digested by NdeI and EcoRI and gel purified to produce recombinant molecule pλcro-ncasTAg1₉₈₇. The insert in the recombinant molecule was verified by DNA sequencing. Recombinant molecule pλcro-rncasTAg1₉₈₇ was used to transform E. coli strain(BL21), thereby producing BL21-pλcro-rncasTAg1₉₈₇.

An overnight culture (25 ml) of BL21-pλcro-rncasTAg1₉₈₇ was grown from 5-10 ml inoculations of frozen stock cultures in Superbroth (3.2% Difco Bacto tryptone, 2% Difco Bacto yeast extract, 0.5% NaCl, 5 mM NaOH, 100 μg/ml ampicillin). Scale-up cultures were produced by inoculating 30 ml of Superbroth with 2-5 ml of cells from the overnight cultures and growing the culture at 32° C. to a cell density of 0.8-1.0 (Absorbance 600 nm (A₆₀₀)).

Production of protein PcasTAg1₃₂₉ was induced by shifting the temperature to 42° C. and incubating for an additional 2-3 hours. Cells were chilled and then harvested by centrifugation at 6000×g for 30 min in an IEC model PR-7000M refrigerated large volume centrifuge, available from International Equipment Company, Needham Heights, Mass.

Harvested cells were suspended (10:1, vol:wt, buffer:cells) in 25 mM Tris, pH 7.5 and passed five times through a Model 1108 microfluidizer, available from Microfluidics Corporation, Newton, Mass., at 120 PSI. The burst cell suspension was centrifuged at 30,000×g for 30 min and the supernatant (S1) was saved for further processing of soluble protein. The pellet (P1) was resuspended in breaking buffer (10:1, vol:wt,) containing 1% triton X and re-centrifuged at 30,000×g for 30 min. The supernatant (S2) was saved for analysis and the washed pellet (P2) was resuspended in breaking buffer (10:1, vol:wt,), and re-centrifuged at 30,000×g for 30 min and the resulting supernatant (S3) and pellet (P3) were saved for analysis. SDS-PAGE and Western blot analyses were performed on samples of S1-S3 and P1-P3 which revealed that the expressed recombinant TAg1 protein (i.e. PcasTAg1₃₂₉) was located exclusively in the insoluble (inclusion bodies) fraction of P3.

Recombinant PcasTAg1₃₂₉ (rPcasTAg1₃₂₉) present in the inclusion bodies was solubilized in 8 M urea, 25 mM Tris, 0.1 M β-mercaptoethanol, pH 9.5, and insoluble contaminants were removed by centrifugation at 30,000×g for 30 min, to produce supernatant S4 and pellet P4. The urea solubilized rPcasTAg1₃₂₉ protein in the S4 supernatant was titrated with 0.1M HCl to a pH of 4.5 and loaded onto a SP-Sepharose column, available from Amersham Pharmacia Biotech, Inc., Piscataway N.J., previously equilibrated with 8 M urea and 25 mM sodium acetate, pH 4.5 and bound protein was eluted with a linear salt gradient to 60% with 1 M NaCl, 25 mM sodium acetate, pH 4.5, in 20 columnar volumes (CV). SDS-PAGE analysis showed that the rPcasTAg1₃₂₉ protein bound the column and eluted at 0.25 NaCl. Fractions containing the rPcasTAg1₃₂₉ protein were pooled, diluted 1:5 (vol:vol) with 25 mM Tris, pH 9.5 and loaded onto an Q-Sepharose column (5 ml HiTrap), available from Amersham Pharmacia Biotech, Inc., Piscataway N.J., previously equilibrated with 25 mM Tris, pH 9.5. Bound protein was eluted with a linear salt gradient to 60% with 1 M NaCl 25 mM Tris, pH 9.5, in 20 CV, with the rPcasTAg1₃₂₉ protein eluting at approximately 0.1 M NaCl. SDS-PAGE analysis showed that the rPcasTAg1₃₂₉ protein was greater than 95% homogeneous and had a weight of approximately 36.8 kDa. N-terminal sequence analysis of the rPcasTAg1₃₂₉ protein gave the sequence MGATGDAEQPRGPGGAERGGS, which matched the predicted amino acid sequence based upon the isolated cDNA described in Example 1.

B. A partial-length (i.e. non-full length) canine TAg1 protein was produced as follows. Nucleic acid molecule ncasTAg1₉₈₇ prepared as described in Example 1 was used as the template in a PCR reaction performed under standard PCR conditions using a sense primer having a nucleic acid sequence, 5′GCG AAT TCC CAT ATG AAC GTT GGT GAA GAA GTG GAC GCA GAG CAG 3′, denoted herein as SEQ ID NO:84, an antisense primer having a nucleic acid sequence 5′CCG TCT GGA ATT CTC AGG TCT GTG GAG GCG CTT CAA C3′, denoted herein as SEQ ID NO:85. The resulting PCR product was a 681-base pair TAg1 nucleic acid molecule representing a C-terminal section of ncasTAg1₉₈₇ spanning from nucleotide 307 through nucleotide 987 of SEQ ID NO:20, having a coding strand denoted herein as SEQ ID NO:73 and a complementary strand denoted herein as SEQ ID NO:75. Translation of SEQ ID NO:73 indicates that ncasTAg1₆₈₁ encodes a 227-amino acid residue protein, denoted herein as SEQ ID NO:74, which corresponds to amino acid residues 103 through 329 of SEQ ID NO:18.

Nucleic acid molecule ncasTAg1₆₈₁ was used as the template to create rncasTAg1₆₈₁, having a coding strand denoted SEQ ID NO:76 and a reverse strand denoted SEQ ID NO:77, by PCR amplification under standard PCR conditions. Nucleic acid molecule rncasTAg1₆₈₁ incorporates E. coli preferred codon changes but encodes a 227 amino acid protein identical to the protein coded by ncasTAg1₆₈₁, (i.e. a protein identical to PcasTag1₆₈₁ SEQ ID NO:74). The following nucleotide changes were incorporated into rncasTAg1₆₈₁: a change from AAT (encoding ASN) to AAC (encoding ASN) at residues corresponding to residues 307 to 309 of SEQ ID NO:71; a change from GTC (encoding VAL) to GTT (encoding VAL) at residues corresponding to residues 310 to 312 of SEQ ID NO:71, and a change from GGG (encoding GLY) to GGT (encoding GLY) at residues corresponding to residues 313 to 315 of SEQ ID NO:71.

To facilitate cloning, an NdeI site (shown in bold in SEQ ID NO:84) was added to the sense primer and an EcoRI site (shown in bold in SEQ ID NO:85) was added to the antisense primer. The resulting PCR fragment was digested with restriction endonucleases NdeI and EcoRI to produce rncasTAg1₆₈₁, gel purified and ligated into λcro plasmid vector that had been digested by NdeI and EcoRI and gel purified to produce recombinant molecule pλcro-ncasTAg1₆₈₁. The insert in the recombinant molecule was verified by DNA sequencing. Recombinant molecule pλcro-rncasTAg1₆₈₁ was used to transform E. coli strain (BL21), thereby producing BL21-pλcro-rncasTAg1₆₈₁. Recombinant protein PcasTAg1₂₂₇ (rPcasTag1₂₂₇) was expressed and purified from BL21-pλcro-rncasTAg1₆₈₁ using the procedures and conditions described in Example 2A. SDS-PAGE analysis showed that the non-full length expressed TAg1 protein rPcasTAg1₂₂₇ was greater than 95% homogeneous and had a weight of approximately 25.7 kDa. N-terminal sequence analysis of the rPcasTAg1₂₂₇ gave the sequence MNVGEEVDAEQLIQEACRS, which matched the predicted amino acid sequence based SEQ ID NO:17.

### Example 3

The following describes the development of monoclonal antibodies that selectively bind to recombinant protein PcasTAg1₂₂₇.

Balb/c mice were immunized in the footpad with about 30 mg recombinant protein PcasTAg1₂₂₇, produced as described in Example 2B, suspended in urea and Freund's complete adjuvant (day 0). Fourteen days later, the mice received a boost of 30 mg of PcasTAg1₂₂₇ in PBS/Freund's incomplete adjuvant (day 14), also administered to the footpad. Sera were tested for the presence of anti-PcasTAg1₂₂₇ antibodies 7-10 days after the second boost (days 21-24). Mice that tested positive were boosted intraveneously with 5 mg PcasTAg1₂₂₇ in PBS at approximately day 28. Splenocytes were harvested from the boosted mice at approximately day 31 and fused with mouse SP2/0 cells (available from ATCC) at mid-log growth phase using polyethylene glycol following established protocols; see, for example, Harlow E., and Lane D., eds., 1995, Antibodies. A Laboratory Manual, Monoclonal Antibodies, Cold Spring Harbor Laboratories; Harlow et al, ibid., is incorporated by reference herein in its entirety.

Fused cells were cultured in RPMI media containing 20% fetal bovine serum, 10% thymocyte conditioned media, 2 mM L-glutamine, 1 mM sodium pyruvate, 60 mM B-mercaptoethanol, all available from Life Technologies Inc., Rockville Md., and hybrids were selected by adding 100 mM hypoxanthine, 10 mM thymidine, and 0.4 mM arninopterin, all available from Sigma Chemical Corporation, St. Louis, Mo.

After about 7 days, wells positive for hybridoma growth were screened by ELISA using recombinant PcasTAg1₂₂₇ protein. Immulon II 96-well plates, available from VWR, Denver, Colo, were incubated overnight with 500 ng/ml rPcasTAg1₂₂₇ in 0.1 M carbonate/bicarbonate buffer, pH 9.2. After blocking the wells with 1% BSA in phosphate buffered saline with Tween (PBST), hybridoma culture supernatants were added and monoclonal antibodies, if present, were allowed to bind to the immobilized rPcasTAg1₂₂₇. Presence of mouse anti-rPcasTAg1₂₂₇ monoclonal antibodies was detected with polyclonal goat anti-mouse IgG conjugated to horseradish peroxidase, available from KPL, Gaithersburg, Md., and color developed with 3,3′,5,5′-tetramethylbenzidine dihydrochloride (TMB), available from Pierce, Rockford, Ill. Specificity of the mouse anti-rPcasTAg1₂₂₇ monoclonal antibodies was verified by Western blot analysisPcasTAg1₂₂₇, developed with polyclonal goat anti-mouse IgG conjugated to horse radish peroxidase and nitro-blue tetrazolium/5-bromo-4-chloro-3′-indolyphosphate p-toluidine salt substrate (NBT/BCIP), available from KPL, Gaithersburg, Md. Immunoglobulin isotyping of the monoclonal antibodies was conducted using IsoStrips, available from Boehringer Mannheim, Indianapolis, Ind. Hybridomas from wells that selectively bound to PcasTAg1₂₂₇ were expanded and dilution cloned by limiting dilution until stable monoclonal antibody secreting lines were identified.

Polyclonal rabbit serum was produced by repeated immunization (over a 10 month period) of a male, New Zealand White rabbit 12-16 months old. Initial immunization was 50 μg rncasTAg1₂₂₇ (prepared as described in Example 2) in Freund's Complete Adjuvant, at several sites subcutaneously and intradermally. One month later, and at one month intervals thereafter, the rabbit was boosted intradermally with 50 ug rncasTAg1₂₂₇ in Freund's Incomplete Adjuvant. Serum was collected bi-weekly and titers monitored by ELISA and Western blot to rncasTAg1₆₂₂₇. Serum that selectively bound to rncasTAg1₆₈₁ protein is referred to as anti-rncasTAg1₂₂₇ serum.

While various embodiments of the present invention have been described in detail, it is apparent that modifications and adaptations of those embodiments will occur to those skilled in the art. It is to be expressly understood, however, that such modifications and adaptations are within the scope of the present invention, as set forth in the following claims.

What is claimed is:

1. An isolated nucleic acid molecule selected from the group consisting of: (a) an isolated canine cDNA or mRNA nucleic acid molecule that hybridizes with a nucleic acid molecule consisting of a nucleic acid sequence selected from the group consisting of SEQ ID NO:8, SEQ ID NO:10, SEQ ID NO:23, SEQ ID NO:25, SEQ ID NO:32, SEQ ID NO:34, SEQ ID NO:35, and SEQ ID NO:37, under conditions comprising (i) hybridizing in a solution comprising 1×SSC in the absence of nucleic acid helix destabilizing agents, at a temperature of about 37° C. and (ii) washing in a solution comprising 1×SSC in the absence of nucleic acid helix destabilizing agents, at a temperature of about 56° C.; (b) an isolated nucleic acid molecule comprising an at least 20 consecutive nucleotide portion identical in sequence to an at least 20 consecutive nucleotide portion of a nucleic acid sequence selected from the group consisting of SEQ ID NO:11, SEQ ID NO:13, SEQ ID NO:26, SEQ ID NO:28, SEQ ID NO:38, SEQ ID NO:40, SEQ ID NO:41, and SEQ ID NO:43; and (c) an isolated nucleic acid molecule comprising an at least 40 consecutive nucleotide portion identical in sequence to an at least 40 consecutive nucleotide portion of a nucleic acid sequence selected from the group consisting of SEQ ID NO:2, SEQ ID NO:4, SEQ ID NO:5, SEQ ID NO:7, SEQ ID NO:14, SEQ ID NO:16, SEQ ID NO:17, SEQ ID NO:19, SEQ ID NO:20, SEQ ID NO:22, SEQ ID NO:29, SEQ ID NO:31, SEQ ID NO:44, SEQ ID NO:46, SEQ ID NO:47, SEQ ID NO:49, SEQ ID NO:50, SEQ ID NO:52, SEQ ID NO:53, SEQ ID NO:55, SEQ ID NO:56, SEQ ID NO:58, SEQ ID NO:59, SEQ ID NO:61, SEQ ID NO:62, SEQ ID NO:64, SEQ ID NO:65, SEQ ID NO:67, SEQ ID NO:68, SEQ ID NO:70, SEQ ID NO:71, SEQ ID NO:72, SEQ ID NO:73, SEQ ID NO:75, SEQ ID NO:76, and SEQ ID NO:77.

2. The nucleic acid molecule of claim 1, said nucleic acid molecule comprising a nucleic acid sequence selected from the group consisting of SEQ ID NO:2, SEQ ID NO:4, SEQ ID NO:5, SEQ ID NO:7, SEQ ID NO:8, SEQ ID NO:10, SEQ ID NO:11, SEQ ID NO:13, SEQ ID NO:14, SEQ ID NO:16, SEQ ID NO:17, SEQ ID NO:19, SEQ ID NO:20, SEQ ID NO:22, SEQ ID NO:23, SEQ ID NO:25, SEQ ID NO:26, SEQ ID NO:28, SEQ ID NO:29, SEQ ID NO:31, SEQ ID NO:32, SEQ ID NO:34, SEQ ID NO:35, SEQ ID NO:37, SEQ ID NO:38, SEQ ID NO:40, SEQ ID NO:41, SEQ ID NO:43, SEQ ID NO:44, SEQ ID NO:46, SEQ ID NO:47, SEQ ID NO:49, SEQ ID NO:50, SEQ ID NO:52, SEQ ID NO:53, SEQ ID NO:55, SEQ ID NO:56, SEQ ID NO:58, SEQ ID NO:59, SEQ ID NO:61, SEQ ID NO:62, SEQ ID NO:64, SEQ ID NO:65, SEQ ID NO:67, SEQ ID NO:68, SEQ ID NO:70, SEQ ID NO:71, SEQ ID NO:72, SEQ ID NO:73, SEQ ID NO:75, SEQ ID NO:76, and SEQ ID NO:77.

3. A recombinant molecule comprising a nucleic acid molecule as set forth in claim 1 operatively linked to a transcription control sequence.

4. A recombinant virus comprising a nucleic acid molecule as set forth in claim 1.

5. A recombinant cell comprising a nucleic acid molecule as set forth in claim 1.

6. A composition comprising an isolated nucleic acid molecule of claim 1 and a component selected from the group consisting of an excipient, an adjuvant and a carrier.

7. A method to produce a protein encoded by an isolated nucleic acid molecule of claim 1, said method comprising culturing a cell transformed with a nucleic acid molecule encoding said protein.

8. The method of claim 7, wherein said nucleic acid molecule is selected from the group consisting of: a nucleic acid molecule that encodes a protein having an amino acid sequence selected from the group consisting of SEQ ID NO:3, SEQ ID NO:9, SEQ ID NO:12, SEQ ID NO:15, SEQ ID NO:18, SEQ ID NO:24, SEQ ID NO:27, SEQ ID NO:30, SEQ ID NO:33, SEQ ID NO:36, SEQ ID NO:39, SEQ ID NO:42, SEQ ID NO:45, SEQ ID NO:48, SEQ ID NO:51, SEQ ID NO:54, SEQ ID NO:57, SEQ ID NO:60, SEQ ID NO:63, SEQ ID NO:66, SEQ ID NO:69, and SEQ ID NO:74.

9. The method of claim 7, wherein said nucleic acid molecule comprises a nucleic acid sequence selected from the group consisting of SEQ ID NO:2, SEQ ID NO:5, SEQ ID NO:8, SEQ ID NO:11, SEQ ID NO:14, SEQ ID NO:17, SEQ ID NO:20, SEQ ID NO:23, SEQ ID NO:26, SEQ ID NO:29, SEQ ID NO:32, SEQ ID NO:35, SEQ ID NO:38, SEQ ID NO:41, SEQ ID NO:44, SEQ ID NO:47, SEQ ID NO:50, SEQ ID NO:53, SEQ ID NO:56, SEQ ID NO:59, SEQ ID NO:62, SEQ ID NO:65, SEQ ID NO:68, SEQ ID NO:71, SEQ ID NO:73, and SEQ ID NO:76.

10. An isolated nucleic acid molecule selected from the group consisting of: (a) an isolated nucleic acid molecule encoding a protein comprising an amino acid sequence selected from the group consisting of SEQ ID NO:3, SEQ ID NO:9, SEQ ID NO:12, SEQ ID NO:15, SEQ ID NO:18, SEQ ID NO:24, SEQ ID NO:27, SEQ ID NO:30, SEQ ID NO:33, SEQ ID NO:36, SEQ ID NO:39, SEQ ID NO:42, SEQ ID NO:45, SEQ ID NO:48, SEQ ID NO:51, SEQ ID NO:54, SEQ ID NO:57, SEQ ID NO:60, SEQ ID NO:63, SEQ ID NO:66, SEQ ID NO:69, and SEQ ID NO:74; (b) an isolated nucleic acid molecule encoding a protein comprising an at least 6 consecutive amino acid portion identical in sequence to an at least 6 consecutive amino acid portion of a sequence selected from the group consisting of SEQ ID NO:9, SEQ ID NO:24, SEQ ID NO:33, and SEQ ID NO:36; (c) an isolated nucleic acid molecule encoding a protein comprising an at least 15 consecutive amino acid portion identical in sequence to an at least 15 consecutive amino acid portion of a sequence selected from the group consisting of SEQ ID NO:12, SEQ ID NO:27, SEQ ID NO:39, and SEQ ID NO:42; (d) an isolated nucleic acid molecule encoding a protein comprising an at least 42 consecutive amino acid portion identical in sequence to an at least 42 consecutive amino acid portion of a sequence selected from the group consisting of SEQ ID NO:3, SEQ ID NO:15, SEQ ID NO:18, SEQ ID NO:30, SEQ ID NO:45, SEQ ID NO:48, SEQ ID NO:51, SEQ ID NO:54, SEQ ID NO:57, SEQ ID NO:60, SEQ ID NO:63, SEQ ID NO:66, SEQ ID NO:69, and SEQ ID NO:74; and (e) a nucleic acid molecule complementary to a nucleic acid molecule of (a), (b), (c) or (d).

11. The nucleic acid molecule of claim 10, wherein said nucleic acid molecule is selected from the group consisting of: a nucleic acid molecule that encodes a protein having an amino acid sequence selected from the group consisting of SEQ ID NO:3, SEQ ID NO:9, SEQ ID NO:12, SEQ ID NO:15, SEQ ID NO:18, SEQ ID NO:24, SEQ ID NO:27, SEQ ID NO:30, SEQ ID NO:33, SEQ ID NO:36, SEQ ID NO:39, SEQ ID NO:42, SEQ ID NO:45, SEQ ID NO:48, SEQ ID NO:51, SEQ ID NO:54, SEQ ID NO:57, SEQ ID NO:60, SEQ ID NO:63, SEQ ID NO:66, SEQ ID NO:69, and SEQ ID NO:74.

12. A composition comprising an isolated nucleic acid molecule of claim 10 and a component selected from the group consisting of an excipient, an adjuvant and a carrier.

13. A method to produce a protein encoded by an isolated nucleic acid molecule of claim 10, said method comprising culturing a cell transformed with a nucleic acid molecule encoding said protein.

14. The method of claim 13, wherein said nucleic acid molecule is selected from the group consisting of: a nucleic acid molecule that encodes a protein having an amino acid sequence selected from the group consisting of SEQ ID NO:3, SEQ ID NO:9, SEQ ID NO:12, SEQ ID NO:15, SEQ ID NO:18, SEQ ID NO:24, SEQ ID NO:27, SEQ ID NO:30, SEQ ID NO:33, SEQ ID NO:36, SEQ ID NO:39, SEQ ID NO:42, SEQ ID NO:45, SEQ ID NO:48, SEQ ID NO:51, SEQ ID NO:54, SEQ ID NO:57, SEQ ID NO:60, SEQ ID NO:63, SEQ ID NO:66, SEQ ID NO:69, and SEQ ID NO:74.

15. The method of claim 13, wherein said nucleic acid molecule comprises a nucleic acid sequence selected from the group consisting of SEQ ID NO:2, SEQ ID NO:5, SEQ ID NO:8, SEQ ID NO:11, SEQ ID NO:14, SEQ ID NO:17, SEQ ID NO:20, SEQ ID NO:23, SEQ ID NO:26, SEQ ID NO:29, SEQ ID NO:32, SEQ ID NO:35, SEQ ID NO:38, SEQ ID NO:41, SEQ ID NO:44, SEQ ID NO:47, SEQ ID NO:50, SEQ ID NO:53, SEQ ID NO:56, SEQ ID NO:59, SEQ ID NO:62, SEQ ID NO:65, SEQ ID NO:68, SEQ ID NO:71, SEQ ID NO:73, and SEQ ID NO:76.

16. An isolated protein selected from the group consisting of: (a) a protein comprising an amino acid sequence selected from the group consisting of SEQ ID NO:3, SEQ ID NO:9, SEQ ID NO:12, SEQ ID NO:15, SEQ ID NO:18, SEQ ID NO:24, SEQ ID NO:27, SEQ ID NO:30, SEQ ID NO:33, SEQ ID NO:36, SEQ ID NO:39, SEQ ID NO:42, SEQ ID NO:45, SEQ ID NO:48, SEQ ID NO:51, SEQ ID NO:54, SEQ ID NO:57, SEQ ID NO:60, SEQ ID NO:63, SEQ ID NO:66, SEQ ID NO:69, and SEQ ID NO:74; (b) a protein comprising an at least 6 consecutive amino acid portion identical in sequence to an at least 6 consecutive amino acid portion of a sequence selected from the group consisting of SEQ ID NO:9, SEQ ID NO:24, SEQ ID NO:33, and SEQ ID NO:36; (c) a protein comprising an at least 15 consecutive amino acid portion identical in sequence to an at least 15 consecutive amino acid portion of a sequence selected from the group consisting of SEQ ID NO:12, SEQ ID NO:27, SEQ ID NO:39, and SEQ ID NO:42 and (d) a protein comprising an at least 42 consecutive amino acid portion identical in sequence to an at least 42 consecutive amino acid portion of a sequence selected from the group consisting of SEQ ID NO:3, SEQ ID NO:15, SEQ ID NO:18, SEQ ID NO:30, SEQ ID NO:45, SEQ ID NO:48, SEQ ID NO:51, SEQ ID NO:54, SEQ ID NO:57, SEQ ID NO:60, SEQ ID NO:63, SEQ ID NO:66, SEQ ID NO:69, and SEQ ID NO:74.

17. The protein of claim 16, wherein said protein is encoded by a nucleic acid molecule having a nucleic acid sequence selected from the group consisting of: SEQ ID NO:2, SEQ ID NO:5, SEQ ID NO:8, SEQ ID NO:11, SEQ ID NO:14, SEQ ID NO:17, SEQ ID NO:20, SEQ ID NO:23, SEQ ID NO:26, SEQ ID NO:29, SEQ ID NO:32, SEQ ID NO:35, SEQ ID NO:38, SEQ ID NO:41, SEQ ID NO:44, SEQ ID NO:47, SEQ ID NO:50, SEQ ID NO:53, SEQ ID NO:56, SEQ ID NO:59, SEQ ID NO:62, SEQ ID NO:65, SEQ ID NO:68, SEQ ID NO:71, SEQ ID NO:73, and SEQ ID NO:76.

18. The protein of claim 16, wherein said protein comprises an amino acid sequence selected from the group consisting of SEQ ID NO:3, SEQ ID NO:9, SEQ ID NO:12, SEQ ID NO:15, SEQ ID NO:18, SEQ ID NO:24, SEQ ID NO:27, SEQ ID NO:30, SEQ ID NO:33, SEQ ID NO:36, SEQ ID NO:39, SEQ ID NO:42, SEQ ID NO:45, SEQ ID NO:48, SEQ ID NO:51, SEQ ID NO:54, SEQ ID NO:57, SEQ ID NO:60, SEQ ID NO:63, SEQ ID NO:66, SEQ ID NO:69, and SEQ ID NO:74.

19. A composition comprising an isolated protein of claim 16 and a component selected from the group consisting of an excipient, an adjuvant and a carrier.

20. An isolated antibody that selectively binds to a protein as set forth in claim 16.

21. A composition comprising an isolated antibody of claim 20 and a component selected from the group consisting of an excipient, an adjuvant and a carrier.

22. A method to detect an inhibitor of TAg1 activity, said method comprising (a) contacting an isolated TAg1 protein of claim 16, with a putative inhibitory compound under conditions in which, in the absence of said compound, said protein has TAg1 protein activity, and (b) determining if said putative inhibitory compound inhibits TAg1 protein activity.

23. A method to detect disease in an animal comprising:
(a) contacting an isolated anti-canine TAg1 antibody of claim 20 with a putative TAg1 protein-containing composition under conditions sufficient to form an immunocomplex between said TAg1 protein and anti-canine TAg1 antibodies; and
(b) measuring immunocomplex formation between said anti-canine TAg1 antibodies and said TAg1 protein, if any, in said fluid, wherein the presence of said immunocomplex indicates the presence of disease.


SEQUENCE LISTING (85 sequences)

SEQ ID NO: 1 - DNA, 814 bp, Mus musculus
ggcccatgagcttccagggatcaagcggggccggcaagcagaagaggagtcccaccgaggaagccccattcccaaaaagaggaaaggtcggcctcctggacacgtcctgtcaaatgaccgcgcagctgctggcatggtatggaaacaaaaatcctgtgaaccaattcgccgagaaggccccaagtgggacccagctcggctgaatgaatctaccacctttgttttggggtctcgagccaacaaggccttggggatgggaggcaccagagggagaatctacatcaagcacccacacctctttaagtatgcagcagatcctcaggacaagcactggctggctgagcagcatcacatgcgggcaacaggcggaaagatggcgtaccttctcattgaagaagacatccgggacttggctgccagcgatgactacagaggatgcttggacctgaagttggaggagctgaaatcctttgttctgccatcctggatggttgagaagatgcggaaatacatggagacactgcggacagaaaatgagcaccgcgctgcggaagcgcctccccagacctgagccgggtgtcctggctactacacttggcggcctgcctccaagaccctctttccccacccggctgaggccatcatggggatttggtctagttgactcttaacagcattaagctgtacatgagctagtttgtagtgactcactgcagagcaccccagactggcatgtggttctgtttctaaagttattggaataagaagcaattaaacagtttgtaataaaaaaagacaaaaaaaattaaaaaaa

SEQ ID NO: 2 - DNA, 1236 bp, Canis familiaris
  CDS (34)..(1011)
gtcgggctgaggttggggggcgccgggggagccatgggggccactggcgacgccgagcagccgcggggacccggcggggcagagcggggcggctccgagctgggcgacgcgggcgcagcggggcagctgaacccttggaacataatgataaagcacaggcaggtgcagcgaaggggccgccgctcacagatgacaacaagtttcacagatcctgccatttccatggacctccttcgagctgtcctgcagcctagcatcaacgaggagatccagacggtcttcaacaagtatatgaagttcttccagaaggcagcactgaacgtcagagacaatgtcggggaagaagtggacgcagagcagctgatccaggaggcctgtcggagctgcctggagcaggctaaactcctgttttcagatggagaaaaagtaatacccagattggcccatgagcttccagggataaagcgtggccgacagacagaagaggaatgtgcccatcgaggaagccctgttcccaaaaagaggaaaggacggcctcctggacacatgctgtcgaatgaccgggcagccgctggcatggtatggaaaccaaagtcctgtgaaccaattcgccgagaaggccccaagtgggacccagcccggctgaatgaatctaccacctttgtgttgggatctcgagccaacaaggccctggggatggggggcaccagagggagaatctacatcaagcacccgcacctctttaagtatgcagctgacccccaggacaagcactggctggccgagcagcatcacatgcgggcaacaggggggaagatggcctacctcctcatcgaggaggacatccgggacctcgcagccagtgatgactatagaggatgcctggacttgaagttggaggagctgaaatcctttgtgctgccctcctggatggttgagaagatgcgaaagtacatggagacactacggacagagaacgagcatcgtgctgttgaagcgcctccacagacctgaccgaagccaagacccctggctacacttggcagccctcctccaaggccctggcacccacgtggcggaggccactgctgggactgcacctggatggatntcagcagcattaagctgtgcctgggctagtctgtagtgactcactacagagcacccccagactggcgtgtggttctgtttgtaaagttatcgggttaagaaacaattaaacagtttgtaattgtg

SEQ ID NO: 3 - protein, 326 aa, Canis familiaris
  misc_feature (1111)..(1111): n = unknown at position 1111
MGATGDAEQPRGPGGAERGGSELGDAGAAGQLNPWNIMIKHRQVQRRGRRSQMTTSFTDPAISMDLLRAVLQPSINEEIQTVFNKYMKFFQKAALNVRDNVGEEVDAEQLIQEACRSCLEQAKLLFSDGEKVIPRLAHELPGIKRGRQTEEECAHRGSPVPKKRKGRPPGHMLSNDRAAAGMVWKPKSCEPIRREGPKWDPARLNESTTFVLGSRANKALGMGGTRGRIYIKHPHLFKYAADPQDKHWLAEQHHMRATGGKMAYLLIEEDIRDLAASDDYRGCLDLKLEELKSFVLPSWMVEKMRKYMETLRTENEHRAVEAPPQT

SEQ ID NO: 4 - DNA, 1236 bp, Canis familiaris
  misc_feature (126)..(126): n = unknown at position 126
cacaattacaaactgtttaattgtttcttaacccgataactttacaaacagaaccacacgccagtctgggggtgctctgtagtgagtcactacagactagcccaggcacagcttaatgctgctganatccatccaggtgcagtcccagcagtggcctccgccacgtgggtgccagggccttggaggagggctgccaagtgtagccaggggtcttggcttcggtcaggtctgtggaggcgcttcaacagcacgatgctcgttctctgtccgtagtgtctccatgtactttcgcatcttctcaaccatccaggagggcagcacaaaggatttcagctcctccaacttcaagtccaggcatcctctatagtcatcactggctgcgaggtcccggatgtcctcctcgatgaggaggtaggccatcttcccccctgttgcccgcatgtgatgctgctcggccagccagtgcttgtcctgggggtcagctgcatacttaaagaggtgcgggtgcttgatgtagattctccctctggtgccccccatccccagggccttgttggctcgagatcccaacacaaaggtggtagattcattcagccgggctgggtcccacttggggccttctcggcgaattggttcacaggactttggtttccataccatgccagcggctgcccggtcattcgacagcatgtgtccaggaggccgtcctttcctctttttgggaacagggcttcctcgatgggcacattcctcttctgtctgtcggccacgctttatccctggaagctcatgggccaatctgggtattactttttctccatctgaaaacaggagtttagcctgctccaggcagctccgacaggcctcctggatcagctgctctgcgtccacttcttccccgacattgtctctgacgttcagtgctgccttctggaagaacttcatatacttgttgaagaccgtctggatctcctcgttgatgctaggctgcaggacagctcgaaggaggtccatggaaatggcaggatctgtgaaacttgttgtcatctgtgagcggcggccccttcgctgcacctgcctgtgctttatcattatgttccaagggttcagctgccccgctgcgcccgcgtcgcccagctcggagccgccccgctctgccccgccgggtccccgcggctgctcggcgtcgccagtggcccccatggctcccccggcgccccccaacctcagcccgac

SEQ ID NO: 5 - DNA, 978 bp, Canis familiaris
  CDS (1)..(978)
atgggggccactggcgacgccgagcagccgcggggacccggcggggcagagcggggcggctccgagctgggcgacgcgggcgcagcggggcagctgaacccttggaacataatgataaagcacaggcaggtgcagcgaaggggccgccgctcacagatgacaacaagtttcacagatcctgccatttccatggacctccttcgagctgtcctgcagcctagcatcaacgaggagatccagacggtcttcaacaagtatatgaagttcttccagaaggcagcactgaacgtcagagacaatgtcggggaagaagtggacgcagagcagctgatccaggaggcctgtcggagctgcctggagcaggctaaactcctgttttcagatggagaaaaagtaatacccagattggcccatgagcttccagggataaagcgtggccgacagacagaagaggaatgtgcccatcgaggaagccctgttcccaaaaagaggaaaggacggcctcctggacacatgctgtcgaatgaccgggcagccgctggcatggtatggaaaccaaagtcctgtgaaccaattcgccgagaaggccccaagtgggacccagcccggctgaatgaatctaccacctttgtgttgggatctcgagccaacaaggccctggggatggggggcaccagagggagaatctacatcaagcacccgcacctctttaagtatgcagctgacccccaggacaagcactggctggccgagcagcatcacatgcgggcaacaggggggaagatggcctacctcctcatcgaggaggacatccgggacctcgcagccagtgatgactatagaggatgcctggacttgaagttggaggagctgaaatcctttgtgctgccctcctggatggttgagaagatgcgaaagtacatggagacactacggacagagaacgagcatcgtgctgttgaagcgcctccacagacc

SEQ ID NO: 6 - protein, 326 aa, Canis familiaris; identical to SEQ ID NO: 3

SEQ ID NO: 7 - DNA, 978 bp, Canis familiaris
ggtctgtggaggcgcttcaacagcacgatgctcgttctctgtccgtagtgtctccatgtactttcgcatcttctcaaccatccaggagggcagcacaaaggatttcagctcctccaacttcaagtccaggcatcctctatagtcatcactggctgcgaggtcccggatgtcctcctcgatgaggaggtaggccatcttcccccctgttgcccgcatgtgatgctgctcggccagccagtgcttgtcctgggggtcagctgcatacttaaagaggtgcgggtgcttgatgtagattctccctctggtgccccccatccccagggccttgttggctcgagatcccaacacaaaggtggtagattcattcagccgggctgggtcccacttggggccttctcggcgaattggttcacaggactttggtttccataccatgccagcggctgcccggtcattcgacagcatgtgtccaggaggccgtcctttcctctttttgggaacagggcttcctcgatgggcacattcctcttctgtctgtcggccacgctttatccctggaagctcatgggccaatctgggtattactttttctccatctgaaaacaggagtttagcctgctccaggcagctccgacaggcctcctggatcagctgctctgcgtccacttcttccccgacattgtctctgacgttcagtgctgccttctggaagaacttcatatacttgttgaagaccgtctggatctcctcgttgatgctaggctgcaggacagctcgaaggaggtccatggaaatggcaggatctgtgaaacttgttgtcatctgtgagcggcggccccttcgctgcacctgcctgtgctttatcattatgttccaagggttcagctgccccgctgcgcccgcgtcgcccagctcggagccgccccgctctgccccgccgggtccccgcggctgctcggcgtcgccagtggcccccat

SEQ ID NO: 8 - DNA, 144 bp, Canis familiaris
  CDS (1)..(144)
atgggggccactggcgacgccgagcagccgcggggacccggcggggcagagcggggcggctccgagctgggcgacgcgggcgcagcggggcagctgaacccttggaacataatgataaagcacaggcaggtgcagcgaaggggc

SEQ ID NO: 9 - protein, 48 aa, Canis familiaris
MGATGDAEQPRGPGGAERGGSELGDAGAAGQLNPWNIMIKHRQVQRRG

SEQ ID NO: 10 - DNA, 144 bp, Canis familiaris
gccccttcgctgcacctgcctgtgctttatcattatgttccaagggttcagctgccccgctgcgcccgcgtcgcccagctcggagccgccccgctctgccccgccgggtccccgcggctgctcggcgtcgccagtggcccccat

SEQ ID NO: 11 - DNA, 222 bp, Canis familiaris
  CDS (1)..(222)
cgccgctcacagatgacaacaagtttcacagatcctgccatttccatggacctccttcgagctgtcctgcagcctagcatcaacgaggagatccagacggtcttcaacaagtatatgaagttcttccagaaggcagcactgaacgtcagagacaatgtcggggaagaagtggacgcagagcagctgatccaggaggcctgtcggagctgcctggagcaggct

SEQ ID NO: 12 - protein, 74 aa, Canis familiaris
RRSQMTTSFTDPAISMDLLRAVLQPSINEEIQTVFNKYMKFFQKAALNVRDNVGEEVDAEQLIQEACRSCLEQA

SEQ ID NO: 13 - DNA, 222 bp, Canis familiaris
agcctgctccaggcagctccgacaggcctcctggatcagctgctctgcgtccacttcttccccgacattgtctctgacgttcagtgctgccttctggaagaacttcatatacttgttgaagaccgtctggatctcctcgttgatgctaggctgcaggacagctcgaaggaggtccatggaaatggcaggatctgtgaaacttgttgtcatctgtgagcggcg

SEQ ID NO: 14 - DNA, 612 bp, Canis familiaris
  CDS (1)..(612)
aaactcctgttttcagatggagaaaaagtaatacccagattggcccatgagcttccagggataaagcgtggccgacagacagaagaggaatgtgcccatcgaggaagccctgttcccaaaaagaggaaaggacggcctcctggacacatgctgtcgaatgaccgggcagccgctggcatggtatggaaaccaaagtcctgtgaaccaattcgccgagaaggccccaagtgggacccagcccggctgaatgaatctaccacctttgtgttgggatctcgagccaacaaggccctggggatggggggcaccagagggagaatctacatcaagcacccgcacctctttaagtatgcagctgacccccaggacaagcactggctggccgagcagcatcacatgcgggcaacaggggggaagatggcctacctcctcatcgaggaggacatccgggacctcgcagccagtgatgactatagaggatgcctggacttgaagttggaggagctgaaatcctttgtgctgccctcctggatggttgagaagatgcgaaagtacatggagacactacggacagagaacgagcatcgtgctgttgaagcgcctccacagacc

SEQ ID NO: 15 - protein, 204 aa, Canis familiaris
KLLFSDGEKVIPRLAHELPGIKRGRQTEEECAHRGSPVPKKRKGRPPGHMLSNDRAAAGMVWKPKSCEPIRREGPKWDPARLNESTTFVLGSRANKALGMGGTRGRIYIKHPHLFKYAADPQDKHWLAEQHHMRATGGKMAYLLIEEDIRDLAASDDYRGCLDLKLEELKSFVLPSWMVEKMRKYMETLRTENEHRAVEAPPQT

SEQ ID NO: 16 - DNA, 612 bp, Canis familiaris
ggtctgtggaggcgcttcaacagcacgatgctcgttctctgtccgtagtgtctccatgtactttcgcatcttctcaaccatccaggagggcagcacaaaggatttcagctcctccaacttcaagtccaggcatcctctatagtcatcactggctgcgaggtcccggatgtcctcctcgatgaggaggtaggccatcttcccccctgttgcccgcatgtgatgctgctcggccagccagtgcttgtcctgggggtcagctgcatacttaaagaggtgcgggtgcttgatgtagattctccctctggtgccccccatccccagggccttgttggctcgagatcccaacacaaaggtggtagattcattcagccgggctgggtcccacttggggccttctcggcgaattggttcacaggactttggtttccataccatgccagcggctgcccggtcattcgacagcatgtgtccaggaggccgtcctttcctctttttgggaacagggcttcctcgatgggcacattcctcttctgtctgtcggccacgctttatccctggaagctcatgggccaatctgggtattactttttctccatctgaaaacaggagttt

SEQ ID NO: 17 - DNA, 1269 bp, Canis familiaris
  CDS (58)..(1044)
ggccactttccggccggtggcagagtcgggctgaggttggggggcgccgggggagccatgggggccactggcgacgccgagcagccgcggggacccggcggggcagagcggggcggctccgagctgggcgacgcgggcgcagcggggcagctggtactcacgaacccttggaacataatgataaagcacaggcaggtgcagcgaaggggccgccgctcacagatgacaacaagtttcacagatcctgccatttccatggacctccttcgagctgtcctgcagcctagcatcaacgaggagatccagacggtcttcaacaagtatatgaagttcttccagaaggcagcactgaacgtcagagacaatgtcggggaagaagtggacgcagagcagctgatccaggaggcctgtcggagctgcctggagcaggctaaactcctgttttcagatggagaaaaagtaatacccagattggcccatgagcttccagggataaagcgtggccgacagacagaagaggaatgtgcccatcgaggaagccctgttcccaaaaagaggaaaggacggcctcctggacacatgctgtcgaatgaccgggcagccgctggcatggtatggaaaccaaagtcctgtgaaccaattcgccgagaaggccccaagtgggacccagcccggctgaatgaatctaccacctttgtgttgggatctcgagccaacaaggccctggggatggggggcaccagagggagaatctacatcaagcacccgcacctctttaagtatgcagctgacccccaggacaagcactggctggccgagcagcatcacatgcgggcaacaggggggaagatggcctacctcctcatcgaggaggacatccgggacctcgcagccagtgatgactatagaggatgcctggacttgaagttggaggagctgaaatcctttgtgctgccctcctggatggttgagaagatgcgaaagtacatggagacactacggacagagaacgagcatcgtgctgttgaagcgcctccacagacctgaccgaagccaagacccctggctacacttggcagccctcctccaaggccctggcacccacgtggcggaggccactgctgggactgcacctggatggatctcagcagcattaagctgtgcctgggctagtctgtagtgactcactacagagcacccccagactggcgtgtggttctgtttgtaaagttatcgggttaagaaacaattaaacagtttttaatagtg

SEQ ID NO: 18 - protein, 329 aa, Canis familiaris
MGATGDAEQPRGPGGAERGGSELGDAGAAGQLVLTNPWNIMIKHRQVQRRGRRSQMTTSFTDPAISMDLLRAVLQPSINEEIQTVFNKYMKFFQKAALNVRDNVGEEVDAEQLIQEACRSCLEQAKLLFSDGEKVIPRLAHELPGIKRGRQTEEECAHRGSPVPKKRKGRPPGHMLSNDRAAAGMVWKPKSCEPIRREGPKWDPARLNESTTFVLGSRANKALGMGGTRGRIYIKHPHLFKYAADPQDKHWLAEQHHMRATGGKMAYLLIEEDIRDLAASDDYRGCLDLKLEELKSFVLPSWMVEKMRKYMETLRTENEHRAVEAPPQT

SEQ ID NO: 19 - DNA, 1269 bp, Canis familiaris
cactattaaaaactgtttaattgtttcttaacccgataactttacaaacagaaccacacgccagtctgggggtgctctgtagtgagtcactacagactagcccaggcacagcttaatgctgctgagatccatccaggtgcagtcccagcagtggcctccgccacgtgggtgccagggccttggaggagggctgccaagtgtagccaggggtcttggcttcggtcaggtctgtggaggcgcttcaacagcacgatgctcgttctctgtccgtagtgtctccatgtactttcgcatcttctcaaccatccaggagggcagcacaaaggatttcagctcctccaacttcaagtccaggcatcctctatagtcatcactggctgcgaggtcccggatgtcctcctcgatgaggaggtaggccatcttcccccctgttgcccgcatgtgatgctgctcggccagccagtgcttgtcctgggggtcagctgcatacttaaagaggtgcgggtgcttgatgtagattctccctctggtgccccccatccccagggccttgttggctcgagatcccaacacaaaggtggtagattcattcagccgggctgggtcccacttggggccttctcggcgaattggttcacaggactttggtttccataccatgccagcggctgcccggtcattcgacagcatgtgtccaggaggccgtcctttcctctttttgggaacagggcttcctcgatgggcacattcctcttctgtctgtcggccacgctttatccctggaagctcatgggccaatctgggtattactttttctccatctgaaaacaggagtttagcctgctccaggcagctccgacaggcctcctggatcagctgctctgcgtccacttcttccccgacattgtctctgacgttcagtgctgccttctggaagaacttcatatacttgttgaagaccgtctggatctcctcgttgatgctaggctgcaggacagctcgaaggaggtccatggaaatggcaggatctgtgaaacttgttgtcatctgtgagcggcggccccttcgctgcacctgcctgtgctttatcattatgttccaagggttcgtgagtaccagctgccccgctgcgcccgcgtcgcccagctcggagccgccccgctctgccccgccgggtccccgcggctgctcggcgtcgccagtggcccccatggctcccccggcgccccccaacctcagcccgactctgccaccggccggaaagtggcc

SEQ ID NO: 20 - DNA, 987 bp, Canis familiaris
  CDS (1)..(987)
atgggggccactggcgacgccgagcagccgcggggacccggcggggcagagcggggcggctccgagctgggcgacgcgggcgcagcggggcagctggtactcacgaacccttggaacataatgataaagcacaggcaggtgcagcgaaggggccgccgctcacagatgacaacaagtttcacagatcctgccatttccatggacctccttcgagctgtcctgcagcctagcatcaacgaggagatccagacggtcttcaacaagtatatgaagttcttccagaaggcagcactgaacgtcagagacaatgtcggggaagaagtggacgcagagcagctgatccaggaggcctgtcggagctgcctggagcaggctaaactcctgttttcagatggagaaaaagtaatacccagattggcccatgagcttccagggataaagcgtggccgacagacagaagaggaatgtgcccatcgaggaagccctgttcccaaaaagaggaaaggacggcctcctggacacatgctgtcgaatgaccgggcagccgctggcatggtatggaaaccaaagtcctgtgaaccaattcgccgagaaggccccaagtgggacccagcccggctgaatgaatctaccacctttgtgttgggatctcgagccaacaaggccctggggatggggggcaccagagggagaatctacatcaagcacccgcacctctttaagtatgcagctgacccccaggacaagcactggctggccgagcagcatcacatgcgggcaacaggggggaagatggcctacctcctcatcgaggaggacatccgggacctcgcagccagtgatgactatagaggatgcctggacttgaagttggaggagctgaaatcctttgtgctgccctcctggatggttgagaagatgcgaaagtacatggagacactacggacagagaacgagcatcgtgctgttgaagcgcctccacagacc

SEQ ID NO: 21 - protein, 329 aa, Canis familiaris; identical to SEQ ID NO: 18

SEQ ID NO: 22 - DNA, 987 bp, Canis familiaris
ggtctgtggaggcgcttcaacagcacgatgctcgttctctgtccgtagtgtctccatgtactttcgcatcttctcaaccatccaggagggcagcacaaaggatttcagctcctccaacttcaagtccaggcatcctctatagtcatcactggctgcgaggtcccggatgtcctcctcgatgaggaggtaggccatcttcccccctgttgcccgcatgtgatgctgctcggccagccagtgcttgtcctgggggtcagctgcatacttaaagaggtgcgggtgcttgatgtagattctccctctggtgccccccatccccagggccttgttggctcgagatcccaacacaaaggtggtagattcattcagccgggctgggtcccacttggggccttctcggcgaattggttcacaggactttggtttccataccatgccagcggctgcccggtcattcgacagcatgtgtccaggaggccgtcctttcctctttttgggaacagggcttcctcgatgggcacattcctcttctgtctgtcggccacgctttatccctggaagctcatgggccaatctgggtattactttttctccatctgaaaacaggagtttagcctgctccaggcagctccgacaggcctcctggatcagctgctctgcgtccacttcttccccgacattgtctctgacgttcagtgctgccttctggaagaacttcatatacttgttgaagaccgtctggatctcctcgttgatgctaggctgcaggacagctcgaaggaggtccatggaaatggcaggatctgtgaaacttgttgtcatctgtgagcggcggccccttcgctgcacctgcctgtgctttatcattatgttccaagggttcgtgagtaccagctgccccgctgcgcccgcgtcgcccagctcggagccgccccgctctgccccgccgggtccccgcggctgctcggcgtcgccagtggcccccat

SEQ ID NO: 23 - DNA, 153 bp, Canis familiaris
  CDS (1)..(153)
atgggggccactggcgacgccgagcagccgcggggacccggcggggcagagcggggcggctccgagctgggcgacgcgggcgcagcggggcagctggtactcacgaacccttggaacataatgataaagcacaggcaggtgcagcgaaggggc

SEQ ID NO: 24 - protein, 51 aa, Canis familiaris
MGATGDAEQPRGPGGAERGGSELGDAGAAGQLVLTNPWNIMIKHRQVQRRG

SEQ ID NO: 25 - DNA, 153 bp, Canis familiaris
gccccttcgctgcacctgcctgtgctttatcattatgttccaagggttcgtgagtaccagctgccccgctgcgcccgcgtcgcccagctcggagccgccccgctctgccccgccgggtccccgcggctgctcggcgtcgccagtggcccccat

SEQ ID NO: 26 - DNA, 222 bp, Canis familiaris; identical to SEQ ID NO: 11
  CDS (1)..(222)

SEQ ID NO: 27 - protein, 74 aa, Canis familiaris; identical to SEQ ID NO: 12

SEQ ID NO: 28 - DNA, 222 bp, Canis familiaris; identical to SEQ ID NO: 13

SEQ ID NO: 29 - DNA, 612 bp, Canis familiaris; identical to SEQ ID NO: 14
  CDS (1)..(612)

SEQ ID NO: 30 - protein, 204 aa, Canis familiaris; identical to SEQ ID NO: 15

SEQ ID NO: 31 - DNA, 612 bp, Canis familiaris; identical to SEQ ID NO: 16

SEQ ID NO: 32 - DNA, 96 bp, Canis familiaris
  CDS (1)..(96)
atgggggccactggcgacgccgagcagccgcggggacccggcggggcagagcggggcggctccgagctgggcgacgcgggcgcagcggggcagctg

SEQ ID NO: 33 - protein, 32 aa, Canis familiaris
MGATGDAEQPRGPGGAERGGSELGDAGAAGQL

SEQ ID NO: 34 - DNA, 96 bp, Canis familiaris
cagctgccccgctgcgcccgcgtcgcccagctcggagccgccccgctctgccccgccgggtccccgcggctgctcggcgtcgccagtggcccccat

SEQ ID NO: 35 - DNA, 69 bp, Canis familiaris
  CDS (1)..(69)
aacccttggaacataatgataaagcacaggcaggtgcagcgaaggggccgccgctcacagatgacaaca

SEQ ID NO: 36 - protein, 23 aa, Canis familiaris
NPWNIMIKHRQVQRRGRRSQMTT

SEQ ID NO: 37 - DNA, 69 bp, Canis familiaris
tgttgtcatctgtgagcggcggccccttcgctgcacctgcctgtgctttatcattatgttccaagggtt

SEQ ID NO: 38 - DNA, 99 bp, Canis familiaris
  CDS (1)..(99)
agtttcacagatcctgccatttccatggacctccttcgagctgtcctgcagcctagcatcaacgaggagatccagacggtcttcaacaagtatatgaag

SEQ ID NO: 39 - protein, 33 aa, Canis familiaris
SFTDPAISMDLLRAVLQPSINEEIQTVFNKYMK

SEQ ID NO: 40 - DNA, 99 bp, Canis familiaris
cttcatatacttgttgaagaccgtctggatctcctcgttgatgctaggctgcaggacagctcgaaggaggtccatggaaatggcaggatctgtgaaact

SEQ ID NO: 41 - DNA, 99 bp, Canis familiaris
  CDS (1)..(99)
ttcttccagaaggcagcactgaacgtcagagacaatgtcggggaagaagtggacgcagagcagctgatccaggaggcctgtcggagctgcctggagcag

SEQ ID NO: 42 - protein, 33 aa, Canis familiaris
FFQKAALNVRDNVGEEVDAEQLIQEACRSCLEQ

SEQ ID NO: 43 - DNA, 99 bp, Canis familiaris
ctgctccaggcagctccgacaggcctcctggatcagctgctctgcgtccacttcttccccgacattgtctctgacgttcagtgctgccttctggaagaa

SEQ ID NO: 44 - DNA, 69 bp, Canis familiaris
  CDS (1)..(69)
gctaaactcctgttttcagatggagaaaaagtaatacccagattggcccatgagcttccagggataaag

SEQ ID NO: 45 - protein, 23 aa, Canis familiaris
AKLLFSDGEKVIPRLAHELPGIK

SEQ ID NO: 46 - DNA, 69 bp, Canis familiaris
ctttatccctggaagctcatgggccaatctgggtattactttttctccatctgaaaacaggagtttagc

SEQ ID NO: 47 - DNA, 57 bp, Canis familiaris
  CDS (1)..(57)
cgtggccgacagacagaagaggaatgtgcccatcgaggaagccctgttcccaaaaag

SEQ ID NO: 48 - protein, 19 aa, Canis familiaris
RGRQTEEECAHRGSPVPKK

SEQ ID NO: 49 - DNA, 57 bp, Canis familiaris
ctttttgggaacagggcttcctcgatgggcacattcctcttctgtctgtcggccacg

SEQ ID NO: 50 - DNA, 60 bp, Canis familiaris
  CDS (1)..(60)
aggaaaggacggcctcctggacacatgctgtcgaatgaccgggcagccgctggcatggta

SEQ ID NO: 51 - protein, 20 aa, Canis familiaris
RKGRPPGHMLSNDRAAAGMV

SEQ ID NO: 52 - DNA, 60 bp, Canis familiaris
taccatgccagcggctgcccggtcattcgacagcatgtgtccaggaggccgtcctttcct

SEQ ID NO: 53 - DNA, 45 bp, Canis familiaris
  CDS (1)..(45)
tggaaaccaaagtcctgtgaaccaattcgccgagaaggccccaag

SEQ ID NO: 54 - protein, 15 aa, Canis familiaris
WKPKSCEPIRREGPK

SEQ ID NO: 55 - DNA, 45 bp, Canis familiaris
cttggggccttctcggcgaattggttcacaggactttggtttcca

SEQ ID NO: 56 - DNA, 60 bp, Canis familiaris
  CDS (1)..(60)
tgggacccagcccggctgaatgaatctaccacctttgtgttgggatctcgagccaacaag

SEQ ID NO: 57 - protein, 20 aa, Canis familiaris
WDPARLNESTTFVLGSRANK

SEQ ID NO: 58 - DNA, 60 bp, Canis familiaris
cttgttggctcgagatcccaacacaaaggtggtagattcattcagccgggctgggtccca

SEQ ID NO: 59 - DNA, 60 bp, Canis familiaris
  CDS (1)..(60)
gccctggggatggggggcaccagagggagaatctacatcaagcacccgcacctctttaag

SEQ ID NO: 60 - protein, 20 aa, Canis familiaris
ALGMGGTRGRIYIKHPHLFK

SEQ ID NO: 61 - DNA, 60 bp, Canis familiaris
cttaaagaggtgcgggtgcttgatgtagattctccctctggtgccccccatccccagggc

SEQ ID NO: 62 - DNA, 72 bp, Canis familiaris
  CDS (1)..(72)
tatgcagctgacccccaggacaagcactggctggccgagcagcatcacatgcgggcaacaggggggaagatg

SEQ ID NO: 63 - protein, 24 aa, Canis familiaris
YAADPQDKHWLAEQHHMRATGGKM

SEQ ID NO: 64 - DNA, 72 bp, Canis familiaris
catcttcccccctgttgcccgcatgtgatgctgctcggccagccagtgcttgtcctgggggtcagctgcata

SEQ ID NO: 65 - DNA, 54 bp, Canis familiaris
  CDS (1)..(54)
gcctacctcctcatcgaggaggacatccgggacctcgcagccagtgatgactat

SEQ ID NO: 66 - protein, 18 aa, Canis familiaris
AYLLIEEDIRDLAASDDY

SEQ ID NO: 67 - DNA, 54 bp, Canis familiaris
atagtcatcactggctgcgaggtcccggatgtcctcctcgatgaggaggtaggc

SEQ ID NO: 68 - DNA, 138 bp, Canis familiaris
  CDS (1)..(138)
agaggatgcctggacttgaagttggaggagctgaaatcctttgtgctgccctcctggatggttgagaagatgcgaaagtacatggagacactacggacagagaacgagcatcgtgctgttgaagcgcctccacagacc

SEQ ID NO: 69 - protein, 46 aa, Canis familiaris
RGCLDLKLEELKSFVLPSWMVEKMRKYMETLRTENEHRAVEAPPQT

SEQ ID NO: 70 - DNA, 138 bp, Canis familiaris
ggtctgtggaggcgcttcaacagcacgatgctcgttctctgtccgtagtgtctccatgtactttcgcatcttctcaaccatccaggagggcagcacaaaggatttcagctcctccaacttcaagtccaggcatcctct

SEQ ID NO: 71 - DNA, 987 bp, Canis familiaris
atgggtgctactggtgacgctgagcagccaagaggtccaggcggggcagagcggggcggctccgagctgggcgacgcgggcgcagcggggcagctggtactcacgaacccttggaacataatgataaagcacaggcaagttcaacgtcgtggccgccgctcacagatgacaacaagtttcacagatcctgccatttccatggacctccttcgagctgtcctgcagcctagcatcaacgaggagatccagacggtcttcaacaagtatatgaagttcttccagaaggcagcactgaacgtcagagacaatgtcggggaagaagtggacgcagagcagctgatccaggaggcctgtcggagctgcctggagcaggctaaactcctgttttcagatggagaaaaagtaatacccagattggcccatgagcttccagggataaagcgtggccgacagacagaagaggaatgtgcccatcgaggaagccctgttcccaaaaagaggaaaggacggcctcctggacacatgctgtcgaatgaccgggcagccgctggcatggtatggaaaccaaagtcctgtgaaccaattcgccgagaaggccccaagtgggacccagcccggctgaatgaatctaccacctttgtgttgggatctcgagccaacaaggccctggggatggggggcaccagagggagaatctacatcaagcacccgcacctctttaagtatgcagctgacccccaggacaagcactggctggccgagcagcatcacatgcgggcaacaggggggaagatggcctacctcctcatcgaggaggacatccgggacctcgcagccagtgatgactatagaggatgcctggacttgaagttggaggagctgaaatcctttgtgctgccctcctggatggttgagaagatgcgaaagtacatggagacactacggacagagaacgagcatcgtgctgttgaagcgcctccacagacc

SEQ ID NO: 72 - DNA, 987 bp, Canis familiaris
ggtctgtggaggcgcttcaacagcacgatgctcgttctctgtccgtagtgtctccatgtactttcgcatcttctcaaccatccaggagggcagcacaaaggatttcagctcctccaacttcaagtccaggcatcctctatagtcatcactggctgcgaggtcccggatgtcctcctcgatgaggaggtaggccatcttcccccctgttgcccgcatgtgatgctgctcggccagccagtgcttgtcctgggggtcagctgcatacttaaagaggtgcgggtgcttgatgtagattctccctctggtgccccccatccccagggccttgttggctcgagatcccaacacaaaggtggtagattcattcagccgggctgggtcccacttggggccttctcggcgaattggttcacaggactttggtttccataccatgccagcggctgcccggtcattcgacagcatgtgtccaggaggccgtcctttcctctttttgggaacagggcttcctcgatgggcacattcctcttctgtctgtcggccacgctttatccctggaagctcatgggccaatctgggtattactttttctccatctgaaaacaggagtttagcctgctccaggcagctccgacaggcctcctggatcagctgctctgcgtccacttcttccccgacattgtctctgacgttcagtgctgccttctggaagaacttcatatacttgttgaagaccgtctggatctcctcgttgatgctaggctgcaggacagctcgaaggaggtccatggaaatggcaggatctgtgaaacttgttgtcatctgtgagcggcggccacgacgttgaacttgcctgtgctttatcattatgttccaagggttcgtgagtaccagctgccccgctgcgcccgcgtcgcccagctcggagccgccccgctctgccccgcctggacctcttggctgctcagcgtcaccagtagcacccat

SEQ ID NO: 73 - DNA, 681 bp, Canis familiaris
  CDS (1)..(681)
aatgtcggggaagaagtggacgcagagcagctgatccaggaggcctgtcggagctgcctggagcaggctaaactcctgttttcagatggagaaaaagtaatacccagattggcccatgagcttccagggataaagcgtggccgacagacagaagaggaatgtgcccatcgaggaagccctgttcccaaaaagaggaaaggacggcctcctggacacatgctgtcgaatgaccgggcagccgctggcatggtatggaaaccaaagtcctgtgaaccaattcgccgagaaggccccaagtgggacccagcccggctgaatgaatctaccacctttgtgttgggatctcgagccaacaaggccctggggatggggggcaccagagggagaatctacatcaagcacccgcacctctttaagtatgcagctgacccccaggacaagcactggctggccgagcagcatcacatgcgggcaacaggggggaagatggcctacctcctcatcgaggaggacatccgggacctcgcagccagtgatgactatagaggatgcctggacttgaagttggaggagctgaaatcctttgtgctgccctcctggatggttgagaagatgcgaaagtacatggagacactacggacagagaacgagcatcgtgctgttgaagcgcctccacagacc

SEQ ID NO: 74 - protein, 227 aa, Canis familiaris
NVGEEVDAEQLIQEACRSCLEQAKLLFSDGEKVIPRLAHELPGIKRGRQTEEECAHRGSPVPKKRKGRPPGHMLSNDRAAAGMVWKPKSCEPIRREGPKWDPARLNESTTFVLGSRANKALGMGGTRGRIYIKHPHLFKYAADPQDKHWLAEQHHMRATGGKMAYLLIEEDIRDLAASDDYRGCLDLKLEELKSFVLPSWMVEKMRKYMETLRTENEHRAVEAPPQT

SEQ ID NO: 75 - DNA, 681 bp, Canis familiaris
ggtctgtggaggcgcttcaacagcacgatgctcgttctctgtccgtagtgtctccatgtactttcgcatcttctcaaccatccaggagggcagcacaaaggatttcagctcctccaacttcaagtccaggcatcctctatagtcatcactggctgcgaggtcccggatgtcctcctcgatgaggaggtaggccatcttcccccctgttgcccgcatgtgatgctgctcggccagccagtgcttgtcctgggggtcagctgcatacttaaagaggtgcgggtgcttgatgtagattctccctctggtgccccccatccccagggccttgttggctcgagatcccaacacaaaggtggtagattcattcagccgggctgggtcccacttggggccttctcggcgaattggttcacaggactttggtttccataccatgccagcggctgcccggtcattcgacagcatgtgtccaggaggccgtcctttcctctttttgggaacagggcttcctcgatgggcacattcctcttctgtctgtcggccacgctttatccctggaagctcatgggccaatctgggtattactttttctccatctgaaaacaggagtttagcctgctccaggcagctccgacaggcctcctggatcagctgctctgcgtccacttcttccccgacatt

SEQ ID NO: 76 - DNA, 681 bp, Canis familiaris
aacgttggtgaagaagtggacgcagagcagctgatccaggaggcctgtcggagctgcctggagcaggctaaactcctgttttcagatggagaaaaagtaatacccagattggcccatgagcttccagggataaagcgtggccgacagacagaagaggaatgtgcccatcgaggaagccctgttcccaaaaagaggaaaggacggcctcctggacacatgctgtcgaatgaccgggcagccgctggcatggtatggaaaccaaagtcctgtgaaccaattcgccgagaaggccccaagtgggacccagcccggctgaatgaatctaccacctttgtgttgggatctcgagccaacaaggccctggggatggggggcaccagagggagaatctacatcaagcacccgcacctctttaagtatgcagctgacccccaggacaagcactggctggccgagcagcatcacatgcgggcaacaggggggaagatggcctacctcctcatcgaggaggacatccgggacctcgcagccagtgatgactatagaggatgcctggacttgaagttggaggagctgaaatcctttgtgctgccctcctggatggttgagaagatgcgaaagtacatggagacactacggacagagaacgagcatcgtgctgttgaagcgcctccacagacc

SEQ ID NO: 77 - DNA, 681 bp, Canis familiaris
ggtctgtggaggcgcttcaacagcacgatgctcgttctctgtccgtagtgtctccatgtactttcgcatcttctcaaccatccaggagggcagcacaaaggatttcagctcctccaacttcaagtccaggcatcctctatagtcatcactggctgcgaggtcccggatgtcctcctcgatgaggaggtaggccatcttcccccctgttgcccgcatgtgatgctgctcggccagccagtgcttgtcctgggggtcagctgcatacttaaagaggtgcgggtgcttgatgtagattctccctctggtgccccccatccccagggccttgttggctcgagatcccaacacaaaggtggtagattcattcagccgggctgggtcccacttggggccttctcggcgaattggttcacaggactttggtttccataccatgccagcggctgcccggtcattcgacagcatgtgtccaggaggccgtcctttcctctttttgggaacagggcttcctcgatgggcacattcctcttctgtctgtcggccacgctttatccctggaagctcatgggccaatctgggtattactttttctccatctgaaaacaggagtttagcctgctccaggcagctccgacaggcctcctggatcagctgctctgcgtccacttcttcaccaacgtt

SEQ ID NO: 78 - DNA, 54 bp, Artificial sequence
  Synthetic Primer
gttaaacatatgggtgctactggtgacgctgagcagccaagaggtccaggcggg

SEQ ID NO: 79 - DNA, 39 bp, Artificial sequence
  Synthetic Primer
gcggcggccacgacgttgaacttgcctgtgctttatcat

SEQ ID NO: 80 - DNA, 39 bp, Artificial sequence
  Synthetic Primer
atgataaagcacaggcaagttcaacgtcgtggccgccgc

SEQ ID NO: 81 - DNA, 30 bp, Artificial sequence
  Synthetic Primer
atgcacgaattctcaggtctgtggaggcgc

SEQ ID NO: 82 - DNA, 33 bp, Artificial sequence
  Synthetic Primer
gttaaacatatgggtgctactggtgacgctgag

SEQ ID NO: 83 - DNA, 27 bp, Artificial sequence
  Synthetic Primer
atgcacgaattctcaggtctgtggagg

SEQ ID NO: 84 - DNA, 45 bp, Artificial sequence
  Synthetic Primer
gcgaattcccatatgaacgttggtgaagaagtggacgcagagcag

SEQ ID NO: 85 - DNA, 37 bp, Artificial sequence
  Synthetic Primer
ccgtctggaattctcaggtctgtggaggcgcttcaac